SEQUENCE LISTING (48 sequences)

SEQ ID NO: 1 - DNA, 20 bp, Artificial Sequence
  Guide 1 for W chromosome
  misc_feature: Synthetic
gcactaggaaccagcagcag

SEQ ID NO: 2 - DNA, 20 bp, Artificial Sequence
  Guide 2 for W chromosome
  misc_feature: Synthetic
gtagccccaagagggctagg

SEQ ID NO: 3 - DNA, 1460 bp, Artificial Sequence
  Synthetic
tcgggctaacattagacccgttgtgtcttaatcctgccgtcgggtatacatatactaaaagaacctcctttccctcctataaatcggagcgagacaaggcattctttttttattattattttggttgcctgcccatcagatgtaatgaaagttgcacagggtagggctaagaaggtaaaggagttaaattcccccgggaggttagagattccctgtattgtggttgcctgcccattagacgtaacaaaagttgtgaagggtaggactaaggatccaggtaaagaggttagtcccccagggttagagtccctcctcagaagtaaatgtaaaacataaccacggtaattgtcgttgagtgcgcttgtttgattgttaattgtttgtgctgttattgtatgctgattgtttgtgttgttattatacaaagagctcacatgcatcgttaaaatgggcattggatcacctaggggtgagggaattttgaaaaagtcacttttgggctgtgttttaagtcattggaaagaaatagctggatcccctggtggggttgcacagaaggatgatttaagtacgatggatgcctttgaggcaggcactaggaaccagcagcagtggcagatttgtgatcatggaaagaattggtgggagcatacggcaccaggactcagataagatagctagctaaggtgatgaattgtagatggtgtcagataacgaaagaagaaaaacaaatgtaataaagtgttaaatgccaatatgttttttataatgaggcaatatcctgaatggcattagtgtgggatcaatttggcccagatgaccctctgattttaatggtagaaaatgataagagagagagggagaaaggaagagagtgtaaaacgtgttgttcagcatgtagtattggactgagatgcttcaagcgaaacaagaatgagcaggaggaggatttagagatgctagtagccccaagagggctagggggcctcaaaatcatggaacccaactccctggagtagaagggtcaggtaatgagtccaaaagagggactataggaaccatgccagtaacagagaggactcacagccagcatagggtcgtgctgtaagccctgttgaggcagttggaaatgacagttcggtagtagttaaggtacccttttaaattacagatttaaacatttggaaagcagctgctggcagctaccgtgatgatcctaaacgggtagctaatgcttttgaaatgatgattaaaactcaggaactggataggaaagatatggaatttattatgcacatgttgtttgatagtacagaaaaataaatgattcactagaccacacggacccaagtggaggatcaggtaatggcaggggttttgcttggatgtaatgctggaaaggacaagagtcctttcctgtcctggggaggatttagaggtggttagaccttggggaggag

SEQ ID NO: 4 - DNA, 592 bp, Artificial Sequence
  For cloning using the gRNA1, W chromosome, “Left arm”
  misc_feature: Synthetic
tcgggctaacattagacccgttgtgtcttaatcctgccgtcgggtatacatatactaaaagaacctcctttccctcctataaatcggagcgagacaaggcattctttttttattattattttggttgcctgcccatcagatgtaatgaaagttgcacagggtagggctaagaaggtaaaggagttaaattcccccgggaggttagagattccctgtattgtggttgcctgcccattagacgtaacaaaagttgtgaagggtaggactaaggatccaggtaaagaggttagtcccccagggttagagtccctcctcagaagtaaatgtaaaacataaccacggtaattgtcgttgagtgcgcttgtttgattgttaattgtttgtgctgttattgtatgctgattgtttgtgttgttattatacaaagagctcacatgcatcgttaaaatgggcattggatcacctaggggtgagggaattttgaaaaagtcacttttgggctgtgttttaagtcattggaaagaaatagctggatcccctggtggggttgcacagaaggatgatttaagtacgatggatgcctttgaggcag

SEQ ID NO: 5 - DNA, 848 bp, Artificial Sequence
  For cloning using the gRNA1, W chromosome, “Right arm”
  misc_feature: Synthetic
tggcagatttgtgatcatggaaagaattggtgggagcatacggcaccaggactcagataagatagctagctaaggtgatgaattgtagatggtgtcagataacgaaagaagaaaaacaaatgtaataaagtgttaaatgccaatatgttttttataatgaggcaatatcctgaatggcattagtgtgggatcaatttggcccagatgaccctctgattttaatggtagaaaatgataagagagagagggagaaaggaagagagtgtaaaacgtgttgttcagcatgtagtattggactgagatgcttcaagcgaaacaagaatgagcaggaggaggatttagagatgctagtagccccaagagggctagggggcctcaaaatcatggaacccaactccctggagtagaagggtcaggtaatgagtccaaaagagggactataggaaccatgccagtaacagagaggactcacagccagcatagggtcgtgctgtaagccctgttgaggcagttggaaatgacagttcggtagtagttaaggtacccttttaaattacagatttaaacatttggaaagcagctgctggcagctaccgtgatgatcctaaacgggtagctaatgcttttgaaatgatgattaaaactcaggaactggataggaaagatatggaatttattatgcacatgttgtttgatagtacagaaaaataaatgattcactagaccacacggacccaagtggaggatcaggtaatggcaggggttttgcttggatgtaatgctggaaaggacaagagtcctttcctgtcctggggaggatttagaggtggttagaccttggggaggag

SEQ ID NO: 6 - DNA, 962 bp, Artificial Sequence
  For cloning using the gRNA2, W chromosome, “Left arm”
  misc_feature: Synthetic
tcgggctaacattagacccgttgtgtcttaatcctgccgtcgggtatacatatactaaaagaacctcctttccctcctataaatcggagcgagacaaggcattctttttttattattattttggttgcctgcccatcagatgtaatgaaagttgcacagggtagggctaagaaggtaaaggagttaaattcccccgggaggttagagattccctgtattgtggttgcctgcccattagacgtaacaaaagttgtgaagggtaggactaaggatccaggtaaagaggttagtcccccagggttagagtccctcctcagaagtaaatgtaaaacataaccacggtaattgtcgttgagtgcgcttgtttgattgttaattgtttgtgctgttattgtatgctgattgtttgtgttgttattatacaaagagctcacatgcatcgttaaaatgggcattggatcacctaggggtgagggaattttgaaaaagtcacttttgggctgtgttttaagtcattggaaagaaatagctggatcccctggtggggttgcacagaaggatgatttaagtacgatggatgcctttgaggcaggcactaggaaccagcagcagtggcagatttgtgatcatggaaagaattggtgggagcatacggcaccaggactcagataagatagctagctaaggtgatgaattgtagatggtgtcagataacgaaagaagaaaaacaaatgtaataaagtgttaaatgccaatatgttttttataatgaggcaatatcctgaatggcattagtgtgggatcaatttggcccagatgaccctctgattttaatggtagaaaatgataagagagagagggagaaaggaagagagtgtaaaacgtgttgttcagcatgtagtattggactgagatgcttcaagcgaaacaagaatgagcaggaggaggatttagagatgcta

SEQ ID NO: 7 - DNA, 478 bp, Artificial Sequence
  For cloning using the gRNA2, W chromosome, “Right arm”
  misc_feature: Synthetic
gggcctcaaaatcatggaacccaactccctggagtagaagggtcaggtaatgagtccaaaagagggactataggaaccatgccagtaacagagaggactcacagccagcatagggtcgtgctgtaagccctgttgaggcagttggaaatgacagttcggtagtagttaaggtacccttttaaattacagatttaaacatttggaaagcagctgctggcagctaccgtgatgatcctaaacgggtagctaatgcttttgaaatgatgattaaaactcaggaactggataggaaagatatggaatttattatgcacatgttgtttgatagtacagaaaaataaatgattcactagaccacacggacccaagtggaggatcaggtaatggcaggggttttgcttggatgtaatgctggaaaggacaagagtcctttcctgtcctggggaggatttagaggtggttagaccttggggaggag

SEQ ID NO: 8 - DNA, 669 bp, Artificial Sequence
  A “left arm” for the region upstream to the CMV-promoter
  misc_feature: Synthetic
aggcaaggcttgaccgacaattgcatgaagtcgggctaacattagacccgttgtgtcttaatcctgccgtcgggtatacatatactaaaagaacctcctttccctcctataaatcggagcgagacaaggcattctttttttattattattttggttgcctgcccatcagatgtaatgaaagttgcacagggtagggctaagaaggtaaaggagttaaattcccccgggaggttagagattccctgtattgtggttgcctgcccattagacgtaacaaaagttgtgaagggtaggactaaggatccaggtaaagaggttagtcccccagggttagagtccctcctcagaagtaaatgtaaaacataaccacggtaattgtcgttgagtgcgcttgtttgattgttaattgtttgtgctgttattgtatgctgattgtttgtgttgttattatacaaagagctcacatgcatcgttaaaatgggcattggatcacctaggggtgagggaattttgaaaaagtcacttttgggctgtgttttaagtcattggaaagaaatagctggatcccctggtggggttgcacagaaggatgatttaagtacgatggatgcctttgaggcaggcactaggaaccagcagcgatgtacgggccagatatacgcgttgaca

SEQ ID NO: 9 - DNA, 911 bp, Artificial Sequence
  A “right arm” for the region downstream the Neomycin-resistance
  misc_feature: Synthetic
ttctatcgccttcttgacgagttcttctgacagtggcagatttgtgatcatggaaagaattggtgggagcatacggcaccaggactcagataagatagctagctaaggtgatgaattgtagatggtgtcagataacgaaagaagaaaaacaaatgtaataaagtgttaaatgccaatatgttttttataatgaggcaatatcctgaatggcattagtgtgggatcaatttggcccagatgaccctctgattttaatggtagaaaatgataagagagagagggagaaaggaagagagtgtaaaacgtgttgttcagcatgtagtattggactgagatgcttcaagcgaaacaagaatgagcaggaggaggatttagagatgctagtagccccaagagggctagggggcctcaaaatcatggaacccaactccctggagtagaagggtcaggtaatgagtccaaaagagggactataggaaccatgccagtaacagagaggactcacagccagcatagggtcgtgctgtaagccctgttgaggcagttggaaatgacagttcggtagtagttaaggtacccttttaaattacagatttaaacatttggaaagcagctgctggcagctaccgtgatgatcctaaacgggtagctaatgcttttgaaatgatgattaaaactcaggaactggataggaaagatatggaatttattatgcacatgttgtttgatagtacagaaaaataaatgattcactagaccacacggacccaagtggaggatcaggtaatggcaggggttttgcttggatgtaatgctggaaaggacaagagtcctttcctgtcctggggaggatttagaggtggttagaccttggggaggagggttcgaaatgaccgaccaagcgacgccca

SEQ ID NO: 10 - DNA, 911 bp, Artificial Sequence
  A “right arm” for the region downstream the polyA site
  misc_feature: Synthetic
atctgtgtgttggttttttgtgtgtctagacagtggcagatttgtgatcatggaaagaattggtgggagcatacggcaccaggactcagataagatagctagctaaggtgatgaattgtagatggtgtcagataacgaaagaagaaaaacaaatgtaataaagtgttaaatgccaatatgttttttataatgaggcaatatcctgaatggcattagtgtgggatcaatttggcccagatgaccctctgattttaatggtagaaaatgataagagagagagggagaaaggaagagagtgtaaaacgtgttgttcagcatgtagtattggactgagatgcttcaagcgaaacaagaatgagcaggaggaggatttagagatgctagtagccccaagagggctagggggcctcaaaatcatggaacccaactccctggagtagaagggtcaggtaatgagtccaaaagagggactataggaaccatgccagtaacagagaggactcacagccagcatagggtcgtgctgtaagccctgttgaggcagttggaaatgacagttcggtagtagttaaggtacccttttaaattacagatttaaacatttggaaagcagctgctggcagctaccgtgatgatcctaaacgggtagctaatgcttttgaaatgatgattaaaactcaggaactggataggaaagatatggaatttattatgcacatgttgtttgatagtacagaaaaataaatgattcactagaccacacggacccaagtggaggatcaggtaatggcaggggttttgcttggatgtaatgctggaaaggacaagagtcctttcctgtcctggggaggatttagaggtggttagaccttggggaggagcaagtaagatgcttttctgtgctgcaatag

SEQ ID NO: 11 - DNA, 17 bp, Artificial Sequence
  Guide 3 for Z chromosome
  misc_feature: Synthetic
acagacctatgatatgt

SEQ ID NO: 12 - DNA, 17 bp, Artificial Sequence
  Guide 4 for Z chromosome
  misc_feature: Synthetic
cgattatcactcacaag

SEQ ID NO: 13 - DNA, 17 bp, Artificial Sequence
  Guide 5 for Z chromosome
  misc_feature: Synthetic
ctggttagcatggggac

SEQ ID NO: 14 - DNA, 17 bp, Artificial Sequence
  Guide 6 for Z chromosome
  misc_feature: Synthetic
gtaaagagtcagataca

SEQ ID NO: 15 - DNA, 4980 bp, Artificial Sequence
  Synthetic
cctgcaggacatgtggacactctgccaggtactgggggaggatcccctccactgctccctgggagatggcaaacatctagagagggacactgcagggatgctgcatgaacacctccagccttctgcaggctgtgatggggtcagggctgaaggtcaacacaagcaatgactgcttgctgacaatgtgggcattgccgaccacagacctatgatatgtgagtggtgagtggggccaggagagcaggacaggagtgtggactcggaggcgcgggcagaggaggtagctcagagcatgagatatattcgccaggtgtcagtggacttctggcagtgcatactgcagagagctttggttgctggccatctggatgatgacaaaatctcagccacagaccatgtgggttggaagagtcctcaggagatcacctttccacctcctgctaaagcacattccctacagtatgtttcacatgacaccgtgctggcagatttttaatatcatcagagaatgacactccatatcctctctgggcagcctttttcagtgctctgtcacccacaaagtaaagtatttgctcatgttctaatggaacttcctatgttctagtttgtgaccattgaaccttgttctgtcactggacagtgctgaaggagcctggccctatccacttgactcccacactttacatatttacaagcatcgataagacccccctcagtcttctccagactagatagccccaggtctcccaaccattccttttatgggagatgcttcaggcccctcatcacctctgtggccctctgctggactgcctgcagtagttccaggtttttttttttgaactggggagcccagaacttgacacggtattccagatgtggcctcaccagcgcagaggagaggggaaggctcacctccctcgacctggtggccatgttttttttaacagacctcaggataccactggccttcttggcccaagggcacactgctggctcatggcccatgggttggcttccaggactctcaggtccttctccgtagagctcctctcaagcaggacaaccctcagcctgtactgatgtgtgcggtgcggttattcctccccacgtgcaagactctacatctgccattgttaaatctcataagcttcctctctgcccaactctccactgtgtacgggccttagtgaatggcagcacagacttctggtgtgaggatagcattcagaacaagctgctgtaccttcccagtcacagaggtgagggtgactggcttgtactttcccagctttgccttcttgcccttttggaagactggagcgacattggccttctggaggaaacggtcctcaggcacccctcctgttctccatgacctttgaaagatgatcgagagcaccttggcagtcacttctgccagctccttcagcacgcatgagtgcatcccgccagtgcccatggatttgtgttcattgaattagcctaggtgacctctgatcagatccttcttgaccaaggggaagacttcctttccccaggttcttatctccagggcctgggattcccaaggggcaatcttagcaggaaaggatgaagtaaaggaggtgtgcagttacagtgctttcatggtgtcctccattactccaatgaggtcacccacctcattcagcagcagacatacattctcccagtcttcctttttctactgccatactaaagtagtccttcatgttgtccttgactgaaggtcacccaacttgatttccagtgagccttagcattccttgtagcattcatgcatgtcctgacaacattcctattttcttcccaagtggccagagaccttttccacactccagaggctttgttcttccattactgcctttccatgagcttcttgctcatctctgcaggtctcatgccacctttgcccgatttcttagtcttagagatgcaccgatcttgaatttggaagaagtggtgcttgaatgacaaccagctctcttggtcccccttgctttccaaagctctaacccatggaatgcctccatgtatgtctgtgaagaggtcaaagtcagctctcctgaagtcgtggttgcaatcctacaaactgcctgatttctcccatgggagatcccgaactccactatctcacagtcactgcactcaaggcttcccccagccttcacatccccagctacaggaagagaagaggctcgaggctacttgcagctgtctgcggaaggcttctccagcttcctcctcttcatcaggcggcctgttgtaaacacccacaacagcgtcacccatcatagcctgtcccttagtcctcacccataaactttctactcattcatcatgcatcccttggcagagctctgtacattccagctgttccctcacataaagagcaactccaccacctcacaccgacgtctgtctgtcctaaaaagtacagagccatccctgagagcattccagtcatgcgagctgccccaccacgtccctctgctgatcgcactgagatcgcggccctacgaccatccatggatctatgactcttcctgtttactccccgcactgtgtgtgatgttacacaggcacttcagagaagaaaagagagcacaggatgccgatttcccttggcaggaccccaggggccgcacgcagcccccacctctatcagcaggtctgagcaggactggagctgcaggcagagcacagctcagccctgagacacctccatgctgcctcctgcacagcctcggccccacagcccccgggaaggagagcacgggtggggtgccgggagccacgctgcgctctgacacactgcgtgtcccctgctcccagcgcgagctgcagggcccgtgcaggaacgagctgggccccaacgtgaccctcggccccggtcactgctgaacgacagcgttcagctctccaggctgcaaagagcgagccgtgaaatggagcaacacgcgggattcccgggtgtgaattcctaaacggattcgaaggtgcccaacaggcagccactgcgtccgcagctgtgcttcggtacctggagacccgcgagcccgtctgttcccaccagcctccagcagccggtgctcgcggatcaaaagaccgccgtgaaggccgcagggccgtccctgggacggagcgccgcccgcttccgccgccagccgcggctccgccctccccgccgagctgacgctggcagcgcgcagagcgcggagcggagccgcggcgctttccggcggcagcgcccgcacggggctcgccgagggccgcgcagagcgctgtgcccggcgggccgcgtcgccgctccggcggcaccgcgattccggcacggagcgcaacgagcgcccgctgcccggctgcaggacgcggtcggggccacaccgcggcggggccgcagcctgccttccgcgggaccgcacgtccggcctccccgaatttggccccgcagcccccgggctgtcaccgtgctgtcaccgtgccgttcacggtttacgggctggttcggcccggttcggcccggttccgtgactggcgggacagagggattcgtggatccgcgcctccgggaagggaaacgggaccccgaataattaaaaacagcggccacgatctgaggggttacaaatgtactaaaacgtactaaaaacaaacagctgcttaacacctcctattccaaaggcccatcgacaccccttggcaccctcaaagtatccccttctgaggtaatgggtaccaagaatgcacggagcccctgggccagtcacagcagggtgcttttgccagtctgtaccggtgaggcctatttcggccacaaacacagtcaattcttgggaacccccattcaccccatgaatataagttgattctgtcccttggtgactcgagggcgtgacagtgcactgtgcaccagggtccaccagggcctcgtattcctgaggttctgatgtgccacaccacgaacccacacggttcaatacactccatcatcctttccccccatggtgggggcagggtccctctatttgttacctctgtcgttcttagagcggttgccatgtcctgcagcaatgggagcgatcacctctttggggattaatcttggtggttaatctgccttgtaattattttacctgtgcttgaaaatgcccctccaggtgaaagcaaactgcagcctgcattctgtggccaaaggaatggaaaagaaggcgttagcaatgccagtggtggcataccacttggcttcttttgactccagttcatactggagttctaacatgtccagaacagcagcgctcagtgggggtgtgacttcattctggccatggtagtctactgtcagcctccattctgcactggctttatgcactggagcacagggcttatggggagcggcggagggagctgggatggttcagtctgcagatgaggaggctcaggggagaccttattgctctctgtaactccatgaagggaggttgtagtgagctgggggtcggtctcttctctcatgtgactagtggtaggactagagggaatggcctccagttgcagcaggggagattcaggctggatgttaggaaatgggctggagtgggctgcccggggaggtggtggagtcaccgaccctggaggtgctcaaggaatgtttagacaccgtgtttagggacatggtatagtgagaactgttggtgatgggtggacagttgggctggatgatcttgtaggtctttttaaagcttggtgattctttgattcgataagtgcaaagtgttgcatttgggtcagagcaatctcacgggtgtgcacaggctgggagaagaactcctgggagaagaagagagcagccctgcagagaaggacttgacatgaaagacttaacgtcagccagactccctctgctctgccctcatgagcctctcctactctgtaaaggtttgggcccccagcaca

SEQ ID NO: 16 - DNA, 4980 bp, Artificial Sequence
  Synthetic
aggagtggcttctgaggctggaaacaaggtgctcccctcttgtcaaacagccaattccatctagctccaacccatcctccactggccaaagctgagcccatcgctgatggtgatagcatctgtaatagcatctcagaaaggctaaaagcactgcgcagcagctgagagaaaagagtgagggaaaatgggacagaaacagctctgcagccctcagaggcaatgcagaggagaggcaggaggtgctccaggcatggagcattagtttcttgcagtccaggagttacaggtgatggtgcaggctctgtgcagcccagtgatgactgtttcaggatccatgctgcagcctgtgccagagccaggggataggcttggctgcttagtgcaggacgtggagagcccatgctgagtgcaagctcccaaaaggctaggacagaacaggtgtagtggagtagatcttgtactgtggagcagtcttttcctggaggactaacctgtgaagaggaggactgcgctgaagcggttcatgaagaatgaaaccttgtgggagggaccccacactgaaaaaggagaaaaccacgatgaggaaggagcagcagagagctccccagccctgtgtgcactgccaggagagaaggaagtagaaaacgtgggaatgtgcaggtgtaaagctgagtctgggaaaaaggtgggggtggaataaaggtatttttagtcctatacctttctcatcaacctactttaattgtagattggccataaattaaatgttttccccaggtcaagtctgtttcttgcttgtgatggtggatgatctccctgtcctcatcccaatcctgaagctatttcatcatatattctctccctgccctgtttcaggacagggagtgcaagcagtttggcagccagacgaggttaacccagcaccagaggtttagtcacatgcaagtgagagctattgcctcaacttaaatgggagaaaacattttagcaggttgagtctcaccttgtttatccagattatacagccatgccagcacatgggctgcatgcagcctggcccaacccatcctctagccaccccctgccccatactgaaaatgcagcggctctgccctgccaagctgccaagcctggctccaggcactcacccattgcttacaaacaacaaaacgtcagcgtgtgagcgcgccctgtgagctgcaactggcatggggagggcacagtcagcacaaatacatgacctgcaaattttcacatggaatgtatggagttgcaatcagatactcagctcaaaaaatttaatcatggctccttacctgtcttttttttaacctttctcttagctcagagaggtatacctcacttcacaatcatgccgtattcatgtcattgctttctggcagtacgtacgtttgtgaacagctgttttcaagggtgatgcacaggaagagtaaaatttcattaaaaaatctccaaggagcacatggagagctcactcagaagcctcaccgctgccactgaacaagactgatgcattagtttcatgaaatcaaggctatgtgtcccactggtttcattattttgttactctttctttaaaacaaaaaaattgttttattgcttatatacatcaactatgttgttatatattttccaaaggccgctccaaaagtaatgcctcctgtttcattctgttggcccaccgtgtcagaggcagatggtggtacggcagtagagatggaaccttcccaccaatattccattacatgttgccatgtgatagacagcagcagatgggcactctgacagaatggtatctgacatggaagcgtggatggagcaaagttgtgtcactgaattcctccataaggaaatgagcacccactaacattcactgatgcttgctgaacatttctggagaccaaacagtggatgtgagcacagtgaggtggtgggtggtgcatttcagcagtggtgacaatgacgttgggtcacctctgctggtgcaggttttgatgagtgcagcctgtgaggtcttgttcatcaatggagaaaatgcacaactaatggtggtgactgctgaaaaatagtgttttgtagctgagaattttctctgtcaaacagtgctaattgtgctctttgtatctgttgtagtttccatgggaataaataggaggcattacttgcagactgacctacatatatgtggccccaggcaagtcaacagctgtacgaaaagtccatgcttcctcattataaacggaggaaaaaaagttgtttacagctgtaatgggattataaaaggcaaatggggatagtacagtggtgagaaccagatctatgaaggaaaagccctaagaaaaagagagtgcacagatactcttaaccatctaataactgtttcctccatcctacagctcagagttaagacttacagaggactctagtacttagtaagatgaatacgagctaatagtggcaaaaataatcccagtgcctcaacactgacctgggaaaaaggggcatgtatagaccttctgatattgtgatgctgtgtttgtacacttattatctacattttcagaaattaggttaaacttcagaaaactgaagatctccagggcttgtagcagaccctgaccaccagactggtccccgattatcactcacaagctctcaaaccgattgtggctgtttctcagaggcaactctttgcgctctagcccctctataacatggggcaacttcccctgccccaccttcccttcctgtatcttctgaaaagcttgtagccctcaattgtcacgctccagccatatgaatcatcccaccacgtccctgtgttcacaattaccttctagtcttctaattgcaccatggcttccaattcctcctgtttatcacccacgttgcatccattgatgtagaggcacttcagtcgggctatcagcaatgttaccacctgtgaggagccctcttgagatcttttaggacaatttagaggttttccccactgtttcctaaaattacagcattccctgtcccttcttagtgatatagaactccatccccttccaccatcaaacctagcttaagctctggtaaatgatccagccagtctgcttcccaaaactcttgctcagttgcattctatctgatgcctacatgcccaacaccttcaaggcctgtccaagatcatagaacacaaaggcttgatcatgacaacatccatgcagccaatcattcacatgacccattcctctcctcctttccaggtcccaacggccaactgagagttgtgctctgagctcttcagtgtccttccgagggatgtaaagtcccttttaatgttctgcattttctttgtcacagccttctgagacccaacttgaatgtggaggagtggagagtaatcatcccgctctgattatcagatacattagcctcttcttggcacctctggcagacagaagacttccctggagagattacctggatgataactgggggcctcagtgcctcacagcagcaagtccccagttactaagactctacacctctttggtggcgctagttctgatgtagttcttcagttgcttaacatgcttgttgtttcccaagatttgactgctatctgcaccctccctttcttccagccccagagccttatatcatgagctgagaacattctgtgttcaaaatacaagacacatttatgcaatttcgtattctctacccatatttttgtgatagaaattagggctacttatttcccatagtgatttccatgctgcctaccaggttctaagtcaggtcaccctgccagcttcttccaaaccatgctcaggagtgcatcaaatcacacgcagacccaaggaaaaaatgtaccaaacaatattaccacgcatgcaaactcctcagtagctctgtcccatatgctacaattaggtctgtataggcttgttagatcttgtctacttgattagtgttttccagcaggcctacaatccaacctaaactgaacagacattaaagacatgtttcactaatacaaaacaactccacctagttcaacagtctgctcttttgtgtatggtacctgaaactcttctgttcactgaacttatcccaacatttttcctcctaatgcatactttttctagacgtgatgtgaaagtacagtgttataccctggtgctgcattaatttgcataaccacagcacacaaagcataatcataacacacaaaggcctcctatgaaaacatctgcttaaagtcataggtcacttatttattctccctctagttatgtaccagaatgatgtatgttaataaataatagattattaaatccatcaaaaattagggaagagctctagcacaaaggcaaagcaaagaagctttgaagaagtgcaagtaaaatgaattatttccaaacaactagcttctcactgtcccactaagaacaaagactattcttatgcactgctgtccttaaaattatcagctgctttgtgtttatttcatggagtaacacatgcaatattttttacaagaaataagaaaccacagcagcagaagacgaatctgcttttattgaacgcagaaatagatggacactgaaacaacagaagtttctaccatgttttgctgctgtgagagcaaatctgctgttgatcaactattagttttgaaaagtatatgcaactctgctatcaactccttccccacatcacatctcgtttcaaggagaaccttcctgtccaggcaactagccagggattttaattttaacatttgctatctcagataatcttttaatatttctaggccgtgcacataacccacatagccctaactccagatgttcccagcatcacttagcagtagttccacttgaagcataaatacagcactggcacaacaaatgaagacaattcaagcacgcctttaacaagcatacagaacatacagtttta

SEQ ID NO: 17 - DNA, 4980 bp, Artificial Sequence
  Synthetic
acaaattaaatgtattttctcttgatactattttgattataagctattaacttgatttgtaaaagcaccgatattatggttaatgtatgtatggtaactgttgagtcacagcctgaacctctgattgaccacctgcggaaaggtctgggtgagccctgggagcacaggcaaaaacaattcagttgtgcaactggaaggcctctcttagacctcatttaagggctgactgtcactggggaaggatctgattctggagatcccttattggtgaagctttcccctgcaaacctagaatcttctgatgcaagtgagcaatcttcttcccttccctagcagctctctgttgcaccagtccttccatccttgcacctttgttgtaatgcttttcccatggcattgatctgtccagttgctacagtgtagaaacaagaaagtgttttctcaaaacattatacatgcttactatcagatcttacttaacattgataagaagtacacctttcattacaacagtgaacggataggcagcaagcattcagaacaatgaaaaagatctgcgttctattgtaaagcaaacaaaggtgatgacggaacgtttatttgagatacaggcaccatagtacaagatatcaaagaggaattaattcaaccctcccaacaacagttgggaaaaaaaaaggataagctgaggaatgcagccttctccgatttttactctggtgccggaaggaggaggaggagcgggggaaaacccagcggactcagactgcatacgcactctcggaagtacctctgaggcggggtgagaggaagggtggatctaggctccccccgctccacacactcacgtgctcgtgtactggttcaggctgtgacttggcaactcctctacatctatccatctagttagactcaacaagaccatcgtgattcaggagtctgaagatgcttaactctctcagaacgtctagttctcctggcctggtaaatgctatgtttctcatactgcctctctgaagaatgcttcgaatgcttctagtgatgctctaaagttctaaacagaaaaatctggagacagttctggtctttagatagaaaaaatgccaacatgccaaaggatggttacatccttcaagcaaccttgttgcatgctgtacaatagactcatgtaataacttagccgtagtcatcgtatctcttatttacctgttcgttattacattttcctggtactgctttatatttagtcagttgtccttttaagacaaattttttggtgtgtgctaataggcagttaccaaatgttctagagggagggaatataatcagtgagtatgtagatgtatatagatgtataaccagtaagtatacagagaagttagggtggtcttttgagagcatatagctggaaagattatcacatgggaaaaggtaatcagaaataaatggaaaaatgctcaccagtgtcataggctcacaagaaaactccccaaggagcaatctccagtaacagacccttcctcagtggcagtcagcccttaaatggcatctaggagaggtgtagccaggcaccaccccttctgtcacataggtgaattgccttcagctgtgctcctgcagctgactcagtgttcacctcaggtgatcaatcagaggtccagaccatgattcaacatttcccatgcggagggagatgaaacaagactgtcatctcatcttctgaagctgataggagaatggttacaataagtaggtccccttccactggtttagctctgtattactcagtttaaaactttatgtagttaatggagttccatagaatcacatcgctggaaaatgtatgtgatagctatacccaaaaagtgacacagcatcacagcctctaccaggtcctctgcctccagacaggaccttgagctgtgtgatccctttgggatgtttctgtttaagacaggggacacttacacattaggaaactggagtcatgtcggtggaggagaaagtggctgtagaaaaggtaacggacaagaaatccctcactggcttagctgtaaaggtcagaggaaaatcatcttctgttctccatacacatctcctgtaattaggcacctgtgccctttaaaaaagtaaagtaatcaaagagtcatttggtgaaaatagaagccaaacagttacaaaatttagttaatattcaaagacaaaacaactctggttccaaatataaaaccctttgtgtaaacactgcgggaaggtgccaaagagccacctacacaaagagatgccacagagtaatttgcgtaccccaacaataggatcatatatgggcaaaccaaaagaccaacaaagaccccttgcaataatcccttgattggcagaagcaagtgcagcgttagatttagtatgtgtgacaattctgagaataggaaggaatttcactgaaactgtgtgaagatttgtatgttaaatacatataatgagggttatttagctctggggtgtgcatgctatgtggagagatccccatgtgcccagcactgcaatagactaatgtcagctttttaaaccatcatttggtttgaagagtttattatgattttcagaaacagaattatgtattcagtgtctgtacattcttacaagcctgctgttctgtacacatgaaaaagctacttatggtgcaaatcagtatagagaagcagttttgacatacagtggtcatgactgggtgatctgctgtcagaaggtaagacactggtatacagaattgcaaacgtgagatgaagacctcaagtgcaactctgtcatcataccacttctcttgctattatctattcagtgtttgttctaaatattcaggcagaaggcttggtttcacatcaggtgttacatttaagtattctttgcccattttttgctgtttgtatgtgtaacttcagattctcacattgactgtgtagtgtaaatttgcaaatagatgtaacagcttctctttaacatcccatgccaaacttttaatcactttcaatcaatgttggaaatctgcagtacataaattttattttgattcattattcagagaatcagagaaataaggacttttttagaggtttatgtttattttagaaaatataaataaatcttgtatacatagatttccaactagtaaaatttttagctatcgctgtagcatttctatgaggagttttactttttactgtctcttatgaaacttagtggaaacaaagctaggtttcataaaacgtctaatctcatattcatctctctccaaaatatggtgaccttcaaaacaagaacagtttgcttatgtaaacatttcttacattgtattctattacattatcatattcatgaagatactttagaattttgttgctgttagttctgttgtgactggctgcttttctcagcttaagagctctgcagaagtgatgataatatatattctctttgctgagttacaggaatttcctgctttcctctactgtacgttggagcccaggagatcagatccaactaccagaattttatatagctctttgtgatcagtccttccttagttgctgttattcctattgatgcaaacctatttatggtgtattaaatgaagaagatataatgaggacagtctaataatcaaagtttttttctgcagccagtagtacaggtagggtgacatgccttgcataaacagatccagatgaaaggtgtgggtgaggcaataagtcttcaggccctgggctaaggcaggacctgttttgtttcatgagatatgcctaggcctctctatagacagggggagatgtcagagaattctgaagcatcagcagaggctatcttggttttcaggccctgtgctaagtatgagaggaagttgagaacagataatgtgtgcaacaaagttagcaacatgctttttcaaaacagaggaaacgatccataaatactggaacagaacaacctcacagaaatcactgactgtccctttgctggccatggcatcctgaaggttgtgctggtagcactcattgggttgctgtagctgataaggcagtgcaaggacattgggtctgcacctgctatgaacaggcactggggcctccaggagctgctggtattagtcatgggcatcagaggtgaagaaggcacttgtagttctcacagctctggcactgaccgatatttctgctgtagttaatgggattatttgcatgtagtcaacagtttacaggctgatttggctctgctccgcaactagcaggacagagggacccacagatccatgtcctggaaggggataagggaaaaggatagagagatgggcctaaaacaaaacagaagcaacgatctgaggataaacaaagtaatttactaaataagatagtggaatgcatgataacacactatagtacaacataatgcaatgtaattagaactggagctaataaatcaaatataaagagagtgtctgaaagccaaaggccttactctaatgctgtgatgagatggcacccacaaaagagacgagccagatgattgaaagggatgtatggtggtctaccaaggcctgttatctgtttgccctgagcagaaatgatagcagaacagcaaacagtcctctggggaatgtagtagttcttctcttctgggacaggtagctggacctagagcattaactctttaacccccagtacactacatgatgttatgatgtggaataccaataaccaaacatcataaaaccatgacatagtcttatgtcgctgtgttgtaactggaagctgaggatctccactcagtatttctgctgaatgaagcagatctttcagttgaaggagaccaacttaatcatccacttccaaccccttactatggacaggttcaccgttcactagatcaagctgcccagggccccatccaacctagccttgaatgcctccagggatgaggcatccacca

SEQ ID NO: 18 - DNA, 4980 bp, Artificial Sequence
  Synthetic
gaaaatctctgccactgattgttttgcagcttagttgtctagttattcgtgagatgttgattcaaacctttgctcagatcaggcacagaagcagtgctgaactttgtttttccgtagccttgctagtgttgtaactgctgtcttagtgaaaacctgcttatttgcatccccttcccaactttgggaaagttgctgtctgtgattccagaattaggactccctcggttgcaacatgactcagattttcagaccttggcttcagaggcacagtggtctttggtgcagagaaaaatgaggctgagaagaccagggccatgatgagtgaggaagatgcaacagaacaagatagagaagagggaagagttttttaggagctccaccagacatactgtagaagcagtgtagatgcacagtaccagtaatgtactccagccaaccactcggtgaagacccatgaaaacctaatgaggttcagtaaggccaagtgcagggtgctgtacttgggtcggggcaatcccaggtatttatacaaactaggggaagatctccttgagagcagccctgcagagaaggacttgggggtcctggtggatgagaagctggacgtgagccagcagtgtgcacctgcagcccggaaggccaactgtgttctgggctgcattaaagaaggggtgaccagcagggagagggaggtgattgtccccctctactcagctcttgtgaggccccatctggagtactgcatccaggcctggagcccccagtacaggaaggacgtggagctcttggagcgggtccagaggaagaccactaagatgatcagagggctggagcacctctcctatgaggaaaggttgagggaactgtccttgtttagcttagagaagagaaggcttcagggaaacctcagtgcagctttccagtacttggaaggagcacataaacaggaaggggaatggctgtttacgagggtggatagtgacaggataagggggaatggttttaaactgagacgggagagttttaggttagatattaggaggaagtttttcacacagagggtggtgacgcactggaacaggttgcccaaggaggttgtggatgccccatccctggaggcattcaaggccaggctggatgtggctctgggcagcctgctctggtggctggcaaccctccacatagcaggggggttgaaactgcgtgatcattgtagtctttttcaacccaggccattctatgattccatgaagactctgtaaggaccccaatgccatcagctgtccccacagcttcagtacccactgccaccacatttttactgagataggtagtttggtagccttcctcccatcttggataggggagggttaactgctggcatttttgttctttcctgcaatatgtgttttctgcatgcattctcttttcacccaaaattttaatgtggacggactttgaggatattctgcatctgcccaatatttcttacagcctcacatctactgatttatgcacagtatcttatatataatgtatattatattatgtatattatgcactgaatcacatctactgtgtatgcacagtaacttctcctatggtactgtaagcccagaaatcccagatatgtcactacaacgtgtctgctatgtattgcttctgtgagacacagatgtgataatcagaggctgtacacagtagaaatgcatacatatcctactgtgaaatctctgaaatctagccttaatcttggaacagaaatgaatatggtgacatcttgatctgatagaattggttgccagtagcacagctgtaatcatccatgatatgaatcaaaccaagcacaggtaaacaggtgagagaaatcatgaacaattacatgcaaacggaccagctaaaatgtgtttgtttgttgttttttttttttcaggtgattcttgattacagtaagatcagaagctgctacattagcagaccagccactgcactcaaggctgtgattcacagcttgcagacctgacagcagttctgtggaagaggcaggtccctgtcaaccagtttaatcaataaatcagtctcgtgtacacaaataatgttatcctgcaccactgctggtgttacactatttcacccaagtttatcaccagcaaactgagtcttatcgttcctactgtgctttgcttttcttgcttagcaaaacatatcagaaaggttttcagttaaaaaaaaaaaaaaaaaaaaaggattatactgcagccatcaaagagttctgttaaagaagttgtggttcatattcttttcaagttctgaagacaatctgacaatagaggagatgagggtttagtggaggtaaatggagctgtttctgaaagtagggcaccgtcgtgaggtaatctcaccactagatgttgctgtaagcatgatgaggggagaagctggagcacaaaaggtgtaaacccaatggttgatcatgcgcactcggacagaggaaatcttcaaaatgggaaagttagctttgaactgatttggtgggttcccattaaaaatgggggacttactgccttattgtctttatgtacccatttgggggatagatgccatacagaaacaccagagcttacggtgtgaattcattgcacatcttattagatcaacaaaatggaataaaagaatacagaagattactactccattgggcatgtggacgttttacaggcctggataaattagatcttaaaaacaaacaaacaaacaaacaaaaaaactccttgcaaagataatgttatgtaatattagttgcaagaagtaagcaaacacacagaactgggagcagaagcaaagcactaagttattaaagcaagttgcacattttgagttgcattttgccactggttttataaacatgtttagcatgtctggtcagaatttgggcaccaggatgcttttaagatgtctgtctatggaacctgtcagtgctcaagaataacttctgttatttggatgctgcaccaaagaattcagaggaagacgagccaagccagacgttatcatagtcactagtaaagtggttctaagcctaattaagacatgtcagaactatgtgttgtgcaaccaaatcctccaaaagagaaatcagaggtgaacttgtgcaataaatatagaagacacgtaaatcctgaggcagttagctaaccatatgaagccaatcatacctgactgctggacgcaggagactgaaccttacagaccctggagaatcactgtttggcctagttaggcctgaatggaatttaccaagattcatgactttaacatggatcaggtgcaaagaaaagaaggctcagttagttcctacaggctaccagatctttttccacctctgctcagcctggagctgtgggctctacctgcccctgaagtgaagtggcatcaactgcaacactttttgcagaggcaaaaatcatcaagtcgtgcctctgcattttaaggtgatggattccaagagatagattccaaacccaacactgagattcctctggtgatgcagatgactgcttactgggatccttctctctctatagcctaatcccatgcagcaccaaaagttgggcacctgaatactatctatcttcctccatctgacctctgctgctggctcatcacgcatggctcctgagcctgacagagccatggggaaagacatggcctgaccttttcagacagcacgacttcctccagtgtgctgcactaacgacaatattaaaaataacactgcaggaaaacatgctgttttctagcaggctggaggaaaaaaaaggtgttcaaggtgagactctataagatcactctttccaaacacagcagttaactcttttctcacttgaagctgaaattcatcttcagtaggtgacggttacaagcagctctccaccggtaagtgaaagtttgctgggtttttttcaggcacttcaagacacgaaggtaattgctgcaacaccttcctagctcttcctgagtactcagcccaaagctacactcttcagggctactctcctagcacggctctctgtaattagcctggctttctaacttatgaattaacgctgcagctactcaacagggcagctttgcagctatgaactttatgcatacactttttattctgggtggtacaaattcattgctaagaaatcttataaactctatggaacacttttaaaagcctacatgtgattaaaaacaaaaaaacaaaaaacactgatatcacatgctatattctctagaaaagagtattttggaaacaattgtaattcacagctcaggagaaagagcaattgttttcctttgtgctggagctggaattctatgcagtataaatacagatgtccaaacaaactcattaaatggtagtcatagcaaatggaggaaaaaaaaagggggggggggggggaactgaagacactgaagaaattctgctgaaattcatggagctgcaacaagaattgatattgattacttcaccatattccgctctaattaactagcacaacactgactgttgtgaaaacaagcatgtgatttgacccaataagtgtttcttttagtaaccaaaaccaaacaacaacaaaccttctggctgaccagatggaggataaattctaacctgatcattctgacacaaatgaggaattatgtaggttggaactgcattccaaagcaaagcatgtttttggcttagttgaacacacagaattcatttttcttgctgattcttgtaactaagggcaggtatgaattggttcccttaaaagaagtccccagaagccatggtcctcaacaagtcttctcagattagcaaggatgaacgactgagaaatcaaaacccaaattcagatctaagagccttcaacagatttgctaaatgaaactagcaaatactgtagaagttaacttgattttgaaataaaacaggatttc

SEQ ID NO: 19 - DNA, 4980 bp, Artificial Sequence
  Synthetic
aaaggagtttcttgcctgaaaccccttaagattttgtgtattgttgttgccctggtttcagctgggacaggactgatatccttcacagtatctggtatgatgctgtgttttggttttaggagaaaaccagtgctgatggcacaccgaggttgtagttgcgactgagcagtgctgcacagagccaaggatgtttcagctgcccacactggccttcctctaagggggtgtgagggcacatggagctggagggaaccaaaggggtattccacaccatatgtcactgagcaaagacattgctgcccaggcagagaatacagtggtagaaatacgtcacataggtgcccatctaccccagaactgggccagcgaagaacatcagaacaacaagcatgcgaatcaggctgctcagattgatgtggctcaggtagatctcaactgtcaactgtcacccataaaatggatgagacaggagcaaggcctgcctgtccaaactgaccttggggccccgggcacagctgctcccgagcaaggagaggcagagaattgtggaggaaaccctcttccacctagctccaagggtgcacgctcaatgcttggatgaagattcaagttctcaatgaagaactgcattcaaagacatctttggaggtgacccagactgcctctgggatgaatccagaacaaacagaatcttcccagcaacacctttgtttatcatacacgttcagaaatgcagtgcctgcctggtatttttttaaactcatcacagaggaatctctgagtggagcagaggagtgtttcctcttgcttttttcttccccctttctgtaagaaatgcatatgccagtttccccctaaatgttttcaaactccaaattgtttcctgctggtgacattctcctctccagcactgcctacaccccagctgcgtctgataggaaaagcacccagtaccatgctggcatggcatggtcagtgacacccactggttagcatggggacaaactgccagagcagagggctgctgaaacgccgagtgccaactgacactgttcagctgacagcctcacgagagtgctgcgagggttaagtgaggcaacaaaatacaagtactcaaaaatagaatggaatggaatggaataacagagggaatggggaacaggctgcagtgagaaggaaacccccgtgcaccgaccaacccgctcccagtagcctggtccctaccttgccccactccagcagctccacgaggtactcgaagtgcccatgtccggccatccccagcatggcacagctgtcacctgggagcagcgggggtccggcaggcatcgccggcacccacagcctgggctgggctgaggatgggcttggtgggcagctgggtgctgccgtggcagctgagtgccgtcgtgcaggtgggctcggtgctctctgcagcccacggctcgggacccgtggtgggcatactgctgggcaaggggacggctcggatgtgcatcccagagcagggtgcatgatgtgtgcgcagcggcacacagcagcgcacttgtccccgaggcaccacccggtctgatgaaattccaggttttgtttaagatgaaaatgcatgtgggcatacatttgcacatgagaacacctgctaatgaaacacacagagttgtatgtgcagtctccctggctggtggcatgtctgtgggggcagttaggcaggaagaaaagctccaccatctgtttctgtgtcccgggcttggtgctgccactagagatgggatgcacacagcatgcctgcgtgctgccgaggcagctcagtacctgctgctgggggagagcagccctcagggagctgctgcaaagaccccgtgggatggctggtgaacagtgctggagacatggcagatgtcgcacccctgctccacaccaccctccgctgcagctgcctgcacgatgccatttcagcgtgccacggcttgtttcttcctcttgcagtctcaggttgccgcagcagcagctgcctgaggtctggtggcaaagggcagagcacccagcccactgctctccatgagtgagcgggtagggggcacgccgtgcttcgcttctggtatcgggtggcctgtggagcacatcacccctggacagtgaaaacatgtcaaagggtgttttataacagtatagcatatccctttgaggctgaatttcctgagcttatggatatggtagtaatgctataccaggctctctctgcaggttgctcactgaaacaatataccctttctttctcaagaaatgggactcatgaatagctaccaggcctttctgctactagatactgtagaattacatatatcagatggctcattaatcacatagcttgttattgggacagaggcaggttttcagcatttcccacactgcttttctgcaaatggattaggagtaaagagtcagatacacaggggaccatcactcacttcctaggccatgcttcgtagtttaactccatgcaaggtattttcttgctctgatctaactctagatcactaaatggcacttgtgcaggacacttttccactgtcattttggggggtagaagtgtggtcagagccagggaggcttgcagggcccacaccagctgtggcccaagcagctgctgtacaatggctgtatggcagtatcgatgtgaacttcctgataataaacagaactcagcagcaaataaacccagattgttctgatcagtaacgagaggctgtgcaaaaagctgagaaatgtacagcccttcctgctcaccactacagcccttcatgcagcggcctgctgcactagggcctgcttggccagaggcagggctgcagcttgagatacgcagcacccagtaccccagcaggctgccatgtgcttctgtcaggccatgaattgcacaagttggtatagttttatacagtgtcatgcagaagcacaactcttaccttggcacactgatatggaaagcaaagtgtaaaggaactgtatgtttcttggcttgtgagttccattggttctcccaggacagcagtgacagctctagattttctggttgatctcttccatctaagtttttacttctgaatacatctgtgcagcaaagcctgatgcttttttttttccccctcccctgggatcgtgaaatattattttcccatctccataatacagatgatccactcaaacatgcatcctcagtcgcagtcctcagaagggccttttcccaccatacacctacacgctacaccagactttattggtcaccatgccttgagggattctgcctaacacaggcacacaggaagggctgataccagtaattcattgctgaggagagaaggggcacttcttttatagtagagccacagaatcaccaggattggaaaacacctccaagatcatccaatccaaccacccacctaccaccagtatttcccactaaaccatgccccttggtaccacatctaaaagcatcttctgcaccttcacagcttcattgcctttctcaggacgtgctccagggccttggtgtctttcttgtattgagtggcccaaaattgaacacaatactcgaggtgcagcttcaccaaagctgagtgcagaggggtgatcacctctactaggccttgttttaccccttccccctgcatacctagtttaaagccctttcagcgagccctaccagctcctgggctaggatccatttccccctttaagagaggtgaaacccacccgcaggcatcaggccaggcactgagttaaactgctccatcagctaaccaccccaatgccctgatgtcccttctgatagtcctcaggcttctctcagcgacttcatcactgccagcctgagctatccgtaatggataacaaccagagggctgaaccagactagggagtctcctggtaatgtccctaagtaatgaattcatgggggaaatggatctgtgcagatgcactgccatctcacagggctcaactgtcacagacagttgtcacaaaccagagcagcagagagagcagcaaaggagatatcataaatagtggaaaagggtgccagtgtttgttattgactgcttcactgtgtgtttggaaatcacaaagaaaacaacaaaaaaaccccacagaatacacatttccaagcatgcatgcttcaggtaaggactgcggcccgctttaattcccctggaagcgtccagaggcctacacacaaatcaggccacaacaaacaggttccgtgcaagcagtttgtgagttgtatgaaaatagctttcttccataaagcgtggcattgatgggaactgttactagcagatgaaaatctgccatgcacgacacccttaatggttggagtggttcgactgagttttgcattcctcattctgatggatgctggcaaaaccaagatcacccaacacgtaacactgaatatagctcaccaacagcaacgagccacaccagatactgaggcatctccaaggctacgtctggtggaaagaagagacagtctcaccctgcagtgcaaaagcatcagggcttctgcgtaaggtttattgatcccaaagataggcaagttaatattcatcagtttaatagacaggtactcacattactggagatggcatgccaggagagcctctgccaagtgctcactgccagtggcagccggccagtgcacagcttcttggtgctgtggccatctatataacagaaggaagcaattaagcatcacctatcaagataagcaagatcattcaaagagaataagggtacttggttagagctgcagagcacttcctcacgaatgagtttcttccagctacatgcagatgttacaggcagtttgcttgtgttctagggataacacaaacccctcctcatgggctagctgcctgctcaggctgtggtctgtatttttgcacgtgcactgctagcctttacttcacatttccctttact

SEQ ID NO: 20 - DNA, 1653 bp, Photinus pyralis
  misc_feature: luciferase gene (cds) GenBank M15077.1
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 21 - protein, 550 aa, Photinus pyralis
  misc_feature: luciferase protein GenBank AAA29795.1
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 22 - DNA, 558 bp, Artificial Sequence
  Synthetic
  misc_feature: Gaussia princeps luciferase GenBank AY015993.1
atgggagtgaaagttctttttgcccttatttgtattgctgtggccgaggccaaaccaactgaaaacaatgaagatttcaacattgtagctgtagctagcaactttgctacaacggatctcgatgctgaccgtggtaaattgcccggaaaaaaattaccacttgaggtactcaaagaaatggaagccaatgctaggaaagctggctgcactaggggatgtctgatatgcctgtcacacatcaagtgtacacccaaaatgaagaagtttatcccaggaagatgccacacctatgaaggagacaaagaaagtgcacagggaggaataggagaggctattgttgacattcctgaaattcctgggtttaaggatttggaacccatggaacaattcattgcacaagttgacctatgtgtagactgcacaactggatgcctcaaaggtcttgccaatgtgcaatgttctgatttactcaagaaatggctgccacaaagatgtgcaacttttgctagcaaaattcaaggccaagtggacaaaataaagggtgccggtggtgattaa

SEQ ID NO: 23 - protein, 185 aa, Gaussia princeps
  misc_feature: luciferase GenBank AAG54095.1
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 24 - DNA, 4107 bp, Streptococcus pyogenes
  misc_feature: M1 GAS Cas9  protein id AAK33936.1
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 25 - protein, 1368 aa, Streptococcus pyogenes [sequence omitted]

SEQ ID NO: 26 - DNA, 20 bp, Artificial Sequence
  gRNA7 for Z chromosome locus chrZ_42174515_-1
  misc_feature: Synthetic
gtaatacagagctaaaccag

SEQ ID NO: 27 - DNA, 20 bp, Artificial Sequence
  gRNA8 for Z chromosome locus chrZ_9157091_1
  misc_feature: Synthetic
acagacctatgatatgtgag

SEQ ID NO: 28 - DNA, 20 bp, Artificial Sequence
  gRNA9 for Z chromosome locus chrZ_27767602_-1
  misc_feature: Synthetic
gagcttgtgagtgataatcg

SEQ ID NO: 29 - DNA, 20 bp, Artificial Sequence
  gRNA10 for Z chromosome locus chrZ_78779927_1
  misc_feature: Synthetic
gtaaagagtcagatacacag

SEQ ID NO: 30 - DNA, 20 bp, Artificial Sequence
  gRNA11 for Z chromosome locus chrZ_63364946_-1
  misc_feature: Synthetic
cagtgggtactgaagctgtg

SEQ ID NO: 31 - DNA, 1012 bp, Artificial Sequence
  Left arm for gRNA7 integration to Chromosome Z ZNF367_HABP4_ENSGALG00000012629_ENSGALG00000012628_chrZ_42174515_ -1
  misc_feature: Synthetic
gtgaacggataggcagcaagcattcagaacaatgaaaaagatctgcgttctattgtaaagcaaacaaaggtgatgacggaacgtttatttgagatacaggcaccatagtacaagatatcaaagaggaattaattcaaccctcccaacaacagttgggaaaaaaaaaggataagctgaggaatgcagccttctccgatttttactctggtgccggaaggaggaggaggagcgggggaaaacccagcggactcagactgcatacgcactctcggaagtacctctgaggcggggtgagaggaagggtggatctaggctccccccgctccacacactcacgtgctcgtgtactggttcaggctgtgacttggcaactcctctacatctatccatctagttagactcaacaagaccatcgtgattcaggagtctgaagatgcttaactctctcagaacgtctagttctcctggcctggtaaatgctatgtttctcatactgcctctctgaagaatgcttcgaatgcttctagtgatgctctaaagttctaaacagaaaaatctggagacagttctggtctttagatagaaaaaatgccaacatgccaaaggatggttacatccttcaagcaaccttgttgcatgctgtacaatagactcatgtaataacttagccgtagtcatcgtatctcttatttacctgttcgttattacattttcctggtactgctttatatttagtcagttgtccttttaagacaaattttttggtgtgtgctaataggcagttaccaaatgttctagagggagggaatataatcagtgagtatgtagatgtatatagatgtataaccagtaagtatacagagaagttagggtggtcttttgagagcatatagctggaaagattatcacatgggaaaaggtaatcagaaataaatggaaaaatgctcaccagtgtcataggctcacaagaaaactccccaaggagcaatctccagtaacagacccttcctca

SEQ ID NO: 32 - DNA, 1023 bp, Artificial Sequence
  Synthetic
gtcatgtcggtggaggagaaagtggctgtagaaaaggtaacggacaagaaatccctcactggcttagctgtaaaggtcagaggaaaatcatcttctgttctccatacacatctcctgtaattaggcacctgtgccctttaaaaaagtaaagtaatcaaagagtcatttggtgaaaatagaagccaaacagttacaaaatttagttaatattcaaagacaaaacaactctggttccaaatataaaaccctttgtgtaaacactgcgggaaggtgccaaagagccacctacacaaagagatgccacagagtaatttgcgtaccccaacaataggatcatatatgggcaaaccaaaagaccaacaaagaccccttgcaataatcccttgattggcagaagcaagtgcagcgttagatttagtatgtgtgacaattctgagaataggaaggaatttcactgaaactgtgtgaagatttgtatgttaaatacatataatgagggttatttagctctggggtgtgcatgctatgtggagagatccccatgtgcccagcactgcaatagactaatgtcagctttttaaaccatcatttggtttgaagagtttattatgattttcagaaacagaattatgtattcagtgtctgtacattcttacaagcctgctgttctgtacacatgaaaaagctacttatggtgcaaatcagtatagagaagcagttttgacatacagtggtcatgactgggtgatctgctgtcagaaggtaagacactggtatacagaattgcaaacgtgagatgaagacctcaagtgcaactctgtcatcataccacttctcttgctattatctattcagtgtttgttctaaatattcaggcagaaggcttggtttcacatcaggtgttacatttaagtattctttgcccattttttgctgtttgtatgtgtaacttcagattctcacattgactgtgtagtgtaaatttgcaaatagatgtaacagcttctctttaacatcccatgc

SEQ ID NO: 33 - DNA, 1014 bp, Artificial Sequence
  Synthetic
ctctgcatggtcacaacggccccaacagcctcccatcccgatgtcaccaatcagttcactattactggtgagcaaccagtccagcactgcatccccctggtggggctgtctgttacgtggctcaggaagttatcctcagtgcattccaggtgctcctggatcgcctgtagctcaccgtgctacttttccagcaggtgtcagggtggtagaagtcccccagcaggacgagagcctgcgattgtgacagcttctgtagctggagggagaatgcttcatcaacaggctccgcttgatcagtgtccctgtactgataccacaaggcttcttttgctgctcccatcttaactctcaccccatcgtggtgttatacgtgcataacatcatgcagtgcactaagagttaaagagttaatgctccagttccgtcgattgtcaataattccaggtgtacctttctcagaagagaagaactacaaatcccttaagatcttactgttccattttcattctccattcagaaggaaaggtaaaactgcagtgtgtgctccctagcattagagtaatttctcccttagctcgttgccaccattttgtttttaggcccacctccttaccttttccctacacccctcctgtgtctctgagacgtgggtccatgggtccctcaaccatagtagacacaaactcagatcaaaccaagccagagctttgacaacccctaagctttcagcttactcagggttgctctgcagggactgctcttcacactctattccttccttgatatagagggcaatgtctcctctcctccttccttacctgtccctcctcatctgcctgttgctgtcagtggccacacttcagccatgggaatcatcccaccctgcttttgtgaaggaaactacattatggttctcaggtagcacagtggcttccatcacctcctgtttgcttcccaagctgtgtgtgttggtgtagaggcacttcagctgggcagctgaagaactggacgcac

SEQ ID NO: 34 - DNA, 1032 bp, Artificial Sequence
  Synthetic
cacctttccacctcctgctaaagcacattccctacagtatgtttcacatgacaccgtgctggcagatttttaatatcatcagagaatgacactccatatcctctctgggcagcctttttcagtgctctgtcacccacaaagtaaagtatttgctcatgttctaatggaacttcctatgttctagtttgtgaccattgaaccttgttctgtcactggacagtgctgaaggagcctggccctatccacttgactcccacactttacatatttacaagcatcgataagacccccctcagtcttctccagactagatagccccaggtctcccaaccattccttttatgggagatgcttcaggcccctcatcacctctgtggccctctgctggactgcctgcagtagttccaggtttttttttttgaactggggagcccagaacttgacacggtattccagatgtggcctcaccagcgcagaggagaggggaaggctcacctccctcgacctggtggccatgttttttttaacagacctcaggataccactggccttcttggcccaagggcacactgctggctcatggcccatgggttggcttccaggactctcaggtccttctccgtagagctcctctcaagcaggacaaccctcagcctgtactgatgtgtgcggtgcggttattcctccccacgtgcaagactctacatctgccattgttaaatctcataagcttcctctctgcccaactctccactgtgtacgggccttagtgaatggcagcacagacttctggtgtgaggatagcattcagaacaagctgctgtaccttcccagtcacagaggtgagggtgactggcttgtactttcccagctttgccttcttgcccttttggaagactggagcgacattggccttctggaggaaacggtcctcaggcacccctcctgttctccatgacctttgaaagatgatcgagagcaccttggcagtcacttctgccagctccttcagcacgcatgagtgcatcccgc

SEQ ID NO: 35 - DNA, 1025 bp, Artificial Sequence
  Synthetic
gcagtacgtacgtttgtgaacagctgttttcaagggtgatgcacaggaagagtaaaatttcattaaaaaatctccaaggagcacatggagagctcactcagaagcctcaccgctgccactgaacaagactgatgcattagtttcatgaaatcaaggctatgtgtcccactggtttcattattttgttactctttctttaaaacaaaaaaattgttttattgcttatatacatcaactatgttgttatatattttccaaaggccgctccaaaagtaatgcctcctgtttcattctgttggcccaccgtgtcagaggcagatggtggtacggcagtagagatggaaccttcccaccaatattccattacatgttgccatgtgatagacagcagcagatgggcactctgacagaatggtatctgacatggaagcgtggatggagcaaagttgtgtcactgaattcctccataaggaaatgagcacccactaacattcactgatgcttgctgaacatttctggagaccaaacagtggatgtgagcacagtgaggtggtgggtggtgcatttcagcagtggtgacaatgacgttgggtcacctctgctggtgcaggttttgatgagtgcagcctgtgaggtcttgttcatcaatggagaaaatgcacaactaatggtggtgactgctgaaaaatagtgttttgtagctgagaattttctctgtcaaacagtgctaattgtgctctttgtatctgttgtagtttccatgggaataaataggaggcattacttgcagactgacctacatatatgtggccccaggcaagtcaacagctgtacgaaaagtccatgcttcctcattataaacggaggaaaaaaagttgtttacagctgtaatgggattataaaaggcaaatggggatagtacagtggtgagaaccagatctatgaaggaaaagccctaagaaaaagagagtgcacagatactcttaaccatctaataactgtttcctccatcctacagctcagag

SEQ ID NO: 36 - DNA, 1040 bp, Artificial Sequence
  Synthetic
tggcttccaattcctcctgtttatcacccacgttgcatccattgatgtagaggcacttcagtcgggctatcagcaatgttaccacctgtgaggagccctcttgagatcttttaggacaatttagaggttttccccactgtttcctaaaattacagcattccctgtcccttcttagtgatatagaactccatccccttccaccatcaaacctagcttaagctctggtaaatgatccagccagtctgcttcccaaaactcttgctcagttgcattctatctgatgcctacatgcccaacaccttcaaggcctgtccaagatcatagaacacaaaggcttgatcatgacaacatccatgcagccaatcattcacatgacccattcctctcctcctttccaggtcccaacggccaactgagagttgtgctctgagctcttcagtgtccttccgagggatgtaaagtcccttttaatgttctgcattttctttgtcacagccttctgagacccaacttgaatgtggaggagtggagagtaatcatcccgctctgattatcagatacattagcctcttcttggcacctctggcagacagaagacttccctggagagattacctggatgataactgggggcctcagtgcctcacagcagcaagtccccagttactaagactctacacctctttggtggcgctagttctgatgtagttcttcagttgcttaacatgcttgttgtttcccaagatttgactgctatctgcaccctccctttcttccagccccagagccttatatcatgagctgagaacattctgtgttcaaaatacaagacacatttatgcaatttcgtattctctacccatatttttgtgatagaaattagggctacttatttcccatagtgatttccatgctgcctaccaggttctaagtcaggtcaccctgccagcttcttccaaaccatgctcaggagtgcatcaaatcacacgcagacccaaggaaaaaatgtaccaaacaatattaccacgcatgcaaactcc

SEQ ID NO: 37 - DNA, 1024 bp, Artificial Sequence
  Synthetic
ctaccttgccccactccagcagctccacgaggtactcgaagtgcccatgtccggccatccccagcatggcacagctgtcacctgggagcagcgggggtccggcaggcatcgccggcacccacagcctgggctgggctgaggatgggcttggtgggcagctgggtgctgccgtggcagctgagtgccgtcgtgcaggtgggctcggtgctctctgcagcccacggctcgggacccgtggtgggcatactgctgggcaaggggacggctcggatgtgcatcccagagcagggtgcatgatgtgtgcgcagcggcacacagcagcgcacttgtccccgaggcaccacccggtctgatgaaattccaggttttgtttaagatgaaaatgcatgtgggcatacatttgcacatgagaacacctgctaatgaaacacacagagttgtatgtgcagtctccctggctggtggcatgtctgtgggggcagttaggcaggaagaaaagctccaccatctgtttctgtgtcccgggcttggtgctgccactagagatgggatgcacacagcatgcctgcgtgctgccgaggcagctcagtacctgctgctgggggagagcagccctcagggagctgctgcaaagaccccgtgggatggctggtgaacagtgctggagacatggcagatgtcgcacccctgctccacaccaccctccgctgcagctgcctgcacgatgccatttcagcgtgccacggcttgtttcttcctcttgcagtctcaggttgccgcagcagcagctgcctgaggtctggtggcaaagggcagagcacccagcccactgctctccatgagtgagcgggtagggggcacgccgtgcttcgcttctggtatcgggtggcctgtggagcacatcacccctggacagtgaaaacatgtcaaagggtgttttataacagtatagcatatccctttgaggctgaatttcctgagcttatggatatggtagtaatgctataccaggctctctctgcag

SEQ ID NO: 38 - DNA, 1022 bp, Artificial Sequence
  Synthetic
cagctgctgtacaatggctgtatggcagtatcgatgtgaacttcctgataataaacagaactcagcagcaaataaacccagattgttctgatcagtaacgagaggctgtgcaaaaagctgagaaatgtacagcccttcctgctcaccactacagcccttcatgcagcggcctgctgcactagggcctgcttggccagaggcagggctgcagcttgagatacgcagcacccagtaccccagcaggctgccatgtgcttctgtcaggccatgaattgcacaagttggtatagttttatacagtgtcatgcagaagcacaactcttaccttggcacactgatatggaaagcaaagtgtaaaggaactgtatgtttcttggcttgtgagttccattggttctcccaggacagcagtgacagctctagattttctggttgatctcttccatctaagtttttacttctgaatacatctgtgcagcaaagcctgatgcttttttttttccccctcccctgggatcgtgaaatattattttcccatctccataatacagatgatccactcaaacatgcatcctcagtcgcagtcctcagaagggccttttcccaccatacacctacacgctacaccagactttattggtcaccatgccttgagggattctgcctaacacaggcacacaggaagggctgataccagtaattcattgctgaggagagaaggggcacttcttttatagtagagccacagaatcaccaggattggaaaacacctccaagatcatccaatccaaccacccacctaccaccagtatttcccactaaaccatgccccttggtaccacatctaaaagcatcttctgcaccttcacagcttcattgcctttctcaggacgtgctccagggccttggtgtctttcttgtattgagtggcccaaaattgaacacaatactcgaggtgcagcttcaccaaagctgagtgcagaggggtgatcacctctactaggccttgttttaccccttcc

SEQ ID NO: 39 - DNA, 1003 bp, Artificial Sequence
  Synthetic
gggttgaaactgcgtgatcattgtagtctttttcaacccaggccattctatgattccatgaagactctgtaaggaccccaatgccatcagctgtccccacagcttcagtacccactgccaccacatttttactgagataggtagtttggtagccttcctcccatcttggataggggagggttaactgctggcatttttgttctttcctgcaatatgtgttttctgcatgcattctcttttcacccaaaattttaatgtggacggactttgaggatattctgcatctgcccaatatttcttacagcctcacatctactgatttatgcacagtatcttatatataatgtatattatattatgtatattatgcactgaatcacatctactgtgtatgcacagtaacttctcctatggtactgtaagcccagaaatcccagatatgtcactacaacgtgtctgctatgtattgcttctgtgagacacagatgtgataatcagaggctgtacacagtagaaatgcatacatatcctactgtgaaatctctgaaatctagccttaatcttggaacagaaatgaatatggtgacatcttgatctgatagaattggttgccagtagcacagctgtaatcatccatgatatgaatcaaaccaagcacaggtaaacaggtgagagaaatcatgaacaattacatgcaaacggaccagctaaaatgtgtttgtttgttgttttttttttttcaggtgattcttgattacagtaagatcagaagctgctacattagcagaccagccactgcactcaaggctgtgattcacagcttgcagacctgacagcagttctgtggaagaggcaggtccctgtcaaccagtttaatcaataaatcagtctcgtgtacacaaataatgttatcctgcaccactgctggtgttacactatttcacccaagtttatcaccagcaaactgagtcttatcgttcctactgtgctttgcttttcttgc

SEQ ID NO: 40 - DNA, 1003 bp, Artificial Sequence
  Synthetic
cagaaacaccagagcttacggtgtgaattcattgcacatcttattagatcaacaaaatggaataaaagaatacagaagattactactccattgggcatgtggacgttttacaggcctggataaattagatcttaaaaacaaacaaacaaacaaacaaaaaaactccttgcaaagataatgttatgtaatattagttgcaagaagtaagcaaacacacagaactgggagcagaagcaaagcactaagttattaaagcaagttgcacattttgagttgcattttgccactggttttataaacatgtttagcatgtctggtcagaatttgggcaccaggatgcttttaagatgtctgtctatggaacctgtcagtgctcaagaataacttctgttatttggatgctgcaccaaagaattcagaggaagacgagccaagccagacgttatcatagtcactagtaaagtggttctaagcctaattaagacatgtcagaactatgtgttgtgcaaccaaatcctccaaaagagaaatcagaggtgaacttgtgcaataaatatagaagacacgtaaatcctgaggcagttagctaaccatatgaagccaatcatacctgactgctggacgcaggagactgaaccttacagaccctggagaatcactgtttggcctagttaggcctgaatggaatttaccaagattcatgactttaacatggatcaggtgcaaagaaaagaaggctcagttagttcctacaggctaccagatctttttccacctctgctcagcctggagctgtgggctctacctgcccctgaagtgaagtggcatcaactgcaacactttttgcagaggcaaaaatcatcaagtcgtgcctctgcattttaaggtgatggattccaagagatagattccaaacccaacactgagattcctctggtgatgcagatgactgcttactgggatccttctctctctatagcctaatcccatgcagcaccaaaag

SEQ ID NO: 41 - DNA, 200 bp, Artificial Sequence
  For cloning using the gRNA3, Z chromosome, “Left arm”
  misc_feature: Synthetic
cctgcaggacatgtggacactctgccaggtactgggggaggatcccctccactgctccctgggagatggcaaacatctagagagggacactgcagggatgctgcatgaacacctccagccttctgcaggctgtgatggggtcagggctgaaggtcaacacaagcaatgactgcttgctgacaatgtgggcattgccgacc

SEQ ID NO: 42 - DNA, 258 bp, Artificial Sequence
  For cloning using the gRNA3, Z chromosome, “Right arm”
  misc_feature: Synthetic
tggtgagtggggccaggagagcaggacaggagtgtggactcggaggcgcgggcagaggaggtagctcagagcatgagatatattcgccaggtgtcagtggacttctggcagtgcatactgcagagagctttggttgctggccatctggatgatgacaaaatctcagccacagaccatgtgggttggaagagtcctcaggagatcacctttccacctcctgctaaagcacattccctacagtatgtttcacatgacacc

SEQ ID NO: 43 - DNA, 436 bp, Artificial Sequence
  For cloning using the gRNA4, Z chromosome, “Left arm”
  misc_feature: Synthetic
tcaacagctgtacgaaaagtccatgcttcctcattataaacggaggaaaaaaagttgtttacagctgtaatgggattataaaaggcaaatggggatagtacagtggtgagaaccagatctatgaaggaaaagccctaagaaaaagagagtgcacagatactcttaaccatctaataactgtttcctccatcctacagctcagagttaagacttacagaggactctagtacttagtaagatgaatacgagctaatagtggcaaaaataatcccagtgcctcaacactgacctgggaaaaaggggcatgtatagaccttctgatattgtgatgctgtgtttgtacacttattatctacattttcagaaattaggttaaacttcagaaaactgaagatctccagggcttgtagcagaccctgaccaccagactggtccc

SEQ ID NO: 44 - DNA, 521 bp, Artificial Sequence
  For cloning using the gRNA4, Z chromosome, “Right arm”
  misc_feature: Synthetic
tcaaaccgattgtggctgtttctcagaggcaactctttgcgctctagcccctctataacatggggcaacttcccctgccccaccttcccttcctgtatcttctgaaaagcttgtagccctcaattgtcacgctccagccatatgaatcatcccaccacgtccctgtgttcacaattaccttctagtcttctaattgcaccatggcttccaattcctcctgtttatcacccacgttgcatccattgatgtagaggcacttcagtcgggctatcagcaatgttaccacctgtgaggagccctcttgagatcttttaggacaatttagaggttttccccactgtttcctaaaattacagcattccctgtcccttcttagtgatatagaactccatccccttccaccatcaaacctagcttaagctctggtaaatgatccagccagtctgcttcccaaaactcttgctcagttgcattctatctgatgcctacatgcccaacaccttcaaggcctgtccaagatc

SEQ ID NO: 45 - DNA, 505 bp, Artificial Sequence
  For cloning using the gRNA5, Z chromosome, “Left arm”
  misc_feature: Synthetic
gcctgtccaaactgaccttggggccccgggcacagctgctcccgagcaaggagaggcagagaattgtggaggaaaccctcttccacctagctccaagggtgcacgctcaatgcttggatgaagattcaagttctcaatgaagaactgcattcaaagacatctttggaggtgacccagactgcctctgggatgaatccagaacaaacagaatcttcccagcaacacctttgtttatcatacacgttcagaaatgcagtgcctgcctggtatttttttaaactcatcacagaggaatctctgagtggagcagaggagtgtttcctcttgcttttttcttccccctttctgtaagaaatgcatatgccagtttccccctaaatgttttcaaactccaaattgtttcctgctggtgacattctcctctccagcactgcctacaccccagctgcgtctgataggaaaagcacccagtaccatgctggcatggcatggtcagtgacaccca

SEQ ID NO: 46 - DNA, 599 bp, Artificial Sequence
  For cloning using the gRNA5, Z chromosome, “Right arm”
  misc_feature: Synthetic
ctgccagagcagagggctgctgaaacgccgagtgccaactgacactgttcagctgacagcctcacgagagtgctgcgagggttaagtgaggcaacaaaatacaagtactcaaaaatagaatggaatggaatggaataacagagggaatggggaacaggctgcagtgagaaggaaacccccgtgcaccgaccaacccgctcccagtagcctggtccctaccttgccccactccagcagctccacgaggtactcgaagtgcccatgtccggccatccccagcatggcacagctgtcacctgggagcagcgggggtccggcaggcatcgccggcacccacagcctgggctgggctgaggatgggcttggtgggcagctgggtgctgccgtggcagctgagtgccgtcgtgcaggtgggctcggtgctctctgcagcccacggctcgggacccgtggtgggcatactgctgggcaaggggacggctcggatgtgcatcccagagcagggtgcatgatgtgtgcgcagcggcacacagcagcgcacttgtccccgaggcaccacccggtctgatgaaattccaggttttgtttaagatgaaaat

SEQ ID NO: 47 - DNA, 453 bp, Artificial Sequence
  For cloning using the gRNA6, Z chromosome, “Left arm”
  misc_feature: Synthetic
ttgcagtctcaggttgccgcagcagcagctgcctgaggtctggtggcaaagggcagagcacccagcccactgctctccatgagtgagcgggtagggggcacgccgtgcttcgcttctggtatcgggtggcctgtggagcacatcacccctggacagtgaaaacatgtcaaagggtgttttataacagtatagcatatccctttgaggctgaatttcctgagcttatggatatggtagtaatgctataccaggctctctctgcaggttgctcactgaaacaatataccctttctttctcaagaaatgggactcatgaatagctaccaggcctttctgctactagatactgtagaattacatatatcagatggctcattaatcacatagcttgttattgggacagaggcaggttttcagcatttcccacactgcttttctgcaaatggattagga

SEQ ID NO: 48 - DNA, 439 bp, Artificial Sequence
  For cloning using the gRNA6, Z chromosome, “Right arm”
  misc_feature: Synthetic
gggaccatcactcacttcctaggccatgcttcgtagtttaactccatgcaaggtattttcttgctctgatctaactctagatcactaaatggcacttgtgcaggacacttttccactgtcattttggggggtagaagtgtggtcagagccagggaggcttgcagggcccacaccagctgtggcccaagcagctgctgtacaatggctgtatggcagtatcgatgtgaacttcctgataataaacagaactcagcagcaaataaacccagattgttctgatcagtaacgagaggctgtgcaaaaagctgagaaatgtacagcccttcctgctcaccactacagcccttcatgcagcggcctgctgcactagggcctgcttggccagaggcagggctgcagcttgagatacgcagcacccagtaccccagcaggctgccat

(19) United States
(12) Patent Application Publication
(10) Pub. No.: US 2019/0029236 A1
(43) Pub. Date: Jan. 31, 2019

(54) METHODS FOR GENDER DETERMINATION OF AVIAN EMBRYOS IN UNHATCHED EGGS AND MEANS THEREOF

(71) Applicant: EGGXYT LTD, Jerusalem (IL)

(72) Inventor: Daniel OFFEN, Kfar HaRoe (IL)

(21) Appl. No.: 15/996,045

(22) Filed: Jun. 1, 2018

Related U.S. Application Data
(63) Continuation-in-part of application No. PCT/IL2016/051291, filed on Dec. 1, 2016.
(60) Provisional application No. 62/262,409, filed on Dec. 3, 2015.

(51) Int. Cl. A01K 67/027; C12N 15/10; C12Q 1/6879; C12N 15/90; C12N 9/02

(52) U.S. Cl. CPC A01K 67/0275; C12N 15/102; C12Q 1/6879; A01K 2227/30; C12N 9/0069; A01K 2217/07; C12N 15/907

(57) ABSTRACT

The present invention relates to methods of gender determination and identification in avian subjects. More specifically, the invention provides non-invasive methods using transgenic avian animals that comprise at least one reporter gene integrated into at least one gender chromosome Z or W. The transgenic avian animals of the invention are used for gender determination and selection of embryos in unhatched avian eggs.

### FIELD OF THE INVENTION

The present invention relates to methods of gender determination and identification in avian subjects. More specifically, the invention provides non-invasive methods and transgenic avian animals for gender determination and selection of embryos in unhatched avian eggs.

### BACKGROUND ART

References considered to be relevant as background to the presently disclosed subject matter are listed below:
WO 2010/103111
WO 2014/0296707
U.S. Pat. No. 6,244,214
WO 06124456A2
US2014069336A
WO16005539
WO 96/39505
WO 97/49806
Quansah, E., Long, J. A., Donovan, D. M., Becker, S. C., Telugu, B., Foster Frey, J. A., Urwin, N. 2014. Sperm-mediated transgenesis in chicken using a PiggyBac transposon system. Poultry Science Association Meeting Abstract. BARC Poster Day.
Jinek, M., Chylinski, K., Fonfara, I., Hauer, M., Doudna, J. A., & Charpentier, E. (2012). A programmable dual-RNA-guided DNA endonuclease in adaptive bacterial immunity Science, 337(6096), 816-821.
Cong, L., & Zhang, F. (2015). Genome engineering using CRISPR-Cas9 system. Chromosomal Mutagenesis, 197-217.
Véron N., Qu Z., Kipen P A., Hirst C E., Marcelle C. (2015). CRISPR mediated somatic cell genome engineering in the chicken. Dev. Biol. 407(1):68-74. doi: 10.1016/j.ydbio.2015.08.007. Epub 2015 Aug 13.
CA2264450.
Niu, Y., B. Shen, Y. Cui, Y. Chen, J. Wang et al., (2014). Generation of genemodified cynomolgus monkey via cas9/rna-mediated gene targeting in one-cell embryos. Cell, 156(4): 836-843.
Hwang, W. Y., Y. Fu, D. Reyon, M. L. Maeder, S. Q. Tsai et al., (2013). Efficient genome editing in zebrafish using a CRISPR-Cas system. Nat. Biotechnol. 31(3): 227-229.
Nadège, V., Q. Zhengdong, P. A. S. Kipen, C. E. Hirst, M. Christophe et al., (2015). CRISPR mediated somatic cell genome engineering in the chicken. Dev. Biol. 407(1): 68-74.

Bai, Y., L. He, P. Li, K. Xu, S. Shao et al., (2016). Efficient genome editing in chicken DF-1 cells using the CRISPR/Cas9 system. G3 (Bethesda) pii: g3.116.027706. Acknowledgement of the above references herein is not to be inferred as meaning that these are in any way relevant to the patentability of the presently disclosed subject matter.

### BACKGROUND OF THE INVENTION

In the food industry, chicks are culled by billions on a daily basis via suffocation or grinding. The males are terminated since they are not useful for laying eggs or to be bread for meat and the weak or unhealthy females are being terminated as well. A method for in-ovo, or embryo sex-determination prior to hatching is thus highly desired due to both ethical and economic considerations.

Specifically, visually identifying poultry fertile eggs is important to allow removal of unfertile eggs to save hatching costs (by prevention of hatching an unfertile egg), and to lower the bio-security risks involved in the continuation of the incubation of these contamination-prone unfertile eggs alongside the fertile eggs.

Visually identifying egg fertility at an early stage of the embryo, while inside the unhatched egg, involves outer light source candling and can be difficult, virtually impossible in early embryonic stages. An even greater challenge is to identify the sex of embryos, and currently there is no available method for the discrimination between males and females in unhatched eggs that are found fertile. However, identification of fertility at an early embryonic stage and the sex determination of poultry are vital for aviculture, scientific research, and conservation. The determination of sex in young birds by morphological features is extremely challenging for most species. The gender may be determined by individual vent sexing which involves manually squeezing the feces out of the chick, which opens up the chicks' anal vent slightly, allowing to see if the chick has a small “bump”, which would indicate that the chick is a male. However, this method represents high risk of bird injury and mistakes in sex determination, together with cumbersome work conducted manually by trained personal.

Vent sexing or chick sexing is the method of distinguishing the sex of chicken and other hatchlings, usually by a trained person called a chick sexer or chicken sexer. Chicken sexing is practiced mostly by large commercial hatcheries, who have to know the difference between the sexes in order to separate them into sex groups, and in order to take them into different programs, which can include the growing of one group and culling of the other group because due to being a sex which does not meet the commercial needs. (In example, a male hatched from an egg that comes from an egg layer commercial line of breed. That male will not have a good meat yield and will not lay eggs; therefore it will be culled after sexing. After the sexing, the relevant sex will continue its course to serve his or her purpose while the other sex or most of it will be culled within days of hatching being irrelevant to egg production.

In farms that produce eggs, males are unwanted, and chicks of an unwanted sex are killed almost immediately to reduce costs to the breeder. Chicks are moved down a conveyer belt, where chick sexers separate out the males and toss them into a chute where they are usually ground up alive in a meat grinder.

Identification and determination of the fertility of an egg and the sex of the embryos in eggs prior to their hatching, will enable the elimination of unfertile eggs, and the unwanted type of embryos while in their eggs, and thus will immensely reduce incubation costs (which includes the energy and efficiency costs alongside with air pollution and energy consumption). In addition, chicks' suffering will cease and pollution from culling will be prevented. An automated sexing device will additionally result in reduced eggs production costs by eliminating the need for chick sexers, as well as reduce the size of the hatcheries needed since at early stage 50% of the eggs will be reduced deducted from the process, thus reducing the costs of hatching these eggs, and later on the need for any elaborate killing procedures.

In all commercial types of birds intended for breeding, laying, or meat production, there is a need to determine fertility and the sex of the embryo. There are great economic returns; in energy saving, biosecurity risk reduction, garbage disposal, sexing labor costs and sexing errors, culling costs and disposal, and animal welfare.

WO 2010/103111 describes an invasive method comprising a series of steps, among them introducing into the egg a labeled antibody, specifically designed to match a sex-specific antigen on the embryo.

WO 2014/0296707 describes luminance composition designed to serve as a biomarker for quantifying or evaluating efficiency of vaccination being injected into the bird's egg. No sex determination is described or even hinted in this disclosure. In-ovo injection apparatus and detection methods was disclosed by U.S. Pat. No. 6,244,214.

WO 06124456A2 discloses invasive methods of in-ovo sex determining of an avian embryo by determining the presence of an estrogenic steroid compound in a sample of embryonic fluid (e.g., allantoic fluid or blood) from the avian egg. Determining the presence of the compound is done by measuring analytes in samples obtained from said avian egg by competitive immunoassay utilizing fluorescence microscopy.

Spectroscopic approaches were also described, among them US2014069336A which is based on screening the avian embryo feather color (pre-hatching) and determining the sex of the avian embryo, based on the feather color or WO016005539 which disclose a device obtaining a shell-specific spectral response to an incident light signal

Further genetic approaches for this problem include DNA sequencing of DNA samples obtained from fertilized eggs for detecting two specific genes located on the Z and W chromosomes of birds (WO 96/39505), or the use of oligonucleotide probes which hybridize to specific sequences of the female W chromosome (WO 97/49806). These methods are invasive and therefore do not provide a safe strategy.

The clustered regularly interspaced short palindromic repeats (CRISPR)/CRISPR-associated (Cas) system is the state of the art gene editing system, allowing a simple construct design with high success rate (M. Jinek and J. Doudna, 2012).

Niu et al. (2014) injected guide RNA (gRNA) and Cas9 RNA into monkey oocytes to modify three target genes, and Hwang et al. (2013) modified the drd3 and gsk3b genes in zebrafish embryos to obtain a two-locus mutant. Cong and Zhang (2015) have modified the CRISPR system to edit any gene in living cells.

Veron and coworkers (2015), demonstrated that expression levels of somatic cells in chicken embryos were modified by electroporation of CRISPR gRNA plasmids directed against the PAX7 transcription factor (Nadège et al. 2015), Bai and coworkers edited the PPAR-g, ATP synthase epsilon subunit (ATPSE).

Quansah, E. et. al., disclosed sperm mediated transgenesis in chicken using a PiggyBac transposon system. In particular, they disclose that aGFP plasmid and Lipofectamine LTXTM 9LPX) combination had no effect on viability, mobility or fertility of chicken sperm.

Thus, effective and non-invasive methods for sex identification during the egg stage, prior to the hatching of the chick are currently not available. There is therefore a long-felt need for a method enabling accurate and safe sex identification of the embryos in unhatched eggs.

### SUMMARY OF THE INVENTION

A first aspect of the invention relates to a method of gender determination of avian, or avian embryo in an unhatched egg, specifically, a fertilized unhatched egg. In some specific embodiments, the method may comprise the step of:

First, in step (a), providing or obtaining at least one transgenic avian subject or animal comprising at least one exogenous reporter gene integrated into at least one position or location (also referred to herein as locus) in at least one of gender chromosome Z and W. In a second step (b) obtaining at least one fertilized egg from the transgenic avian subject, specifically animal or of any cells thereof.

The next step (c) involves determining in the egg if at least one detectable signal is detected. In more specific embodiments, detection of at least one detectable signal indicates the expression of said at least one reporter gene, thereby the presence of the W chromosome or Z chromosome in the avian embryo.

In a second aspect, the invention relates to an avian transgenic animal comprising, in at least one cell thereof, at least one exogenous reporter gene integrated into at least one position or location (also referred to herein as locus) in at least one of gender chromosome Z and W.

In yet another aspect, the invention relates to a cell comprising at least one exogenous reporter gene integrated into at least one position or locus in at least one of gender chromosome Z and W.

In yet a further aspect thereof, the invention relates to any egg derived, laid or fertilized by at least one of any of the transgenic avian subjects or animals of the invention, or by any progeny thereof, any component or any parts thereof or any product comprising said egg, components or parts thereof. It should be understood that in some embodiments, such transgenic avian subjects may comprise, in at least one cell thereof, at least one exogenous reporter gene integrated into at least one position or location (also referred to herein as locus) in at least one of gender chromosome Z and W.

In yet a further aspect, the invention provides a kit comprising:

(a) at least one first nucleic acid sequence comprising at least one nucleic acid sequence encoding at least one Cas9 protein and at least one nucleic acid sequence encoding at least one guide RNA (gRNA); and (b) at least one second nucleic acid sequence comprising at least one said reporter gene.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B. Luciferase Reporter Gene Signal Penetrates the Egg Shell

Luciferase expressing transgenic mice were injected subcutaneously with luciferin. Ear (FIG. 1A) and tail (FIG. 1B) are excised 10 min thereafter and incorporated into unfertilized eggs. Eggs were imaged using the bio-space photon Imager (Bio space lab, USA).

FIGS. 2A-2B. Luciferase Reporter Gene Signal is Formed in a Fertilized Egg and Penetrates the Egg Shell

Ear (FIG. 2A) and tail (FIG. 2B), excised from luciferase expressing transgenic mice, were incorporated into a fertilized carrying a 10 days old chicken embryo. Luciferin is subsequently injected to induce bioluminescence. Images were taken 10 minutes thereafter using the bio-space photon Imager (Bio space lab, USA).

FIGS. 3A-3B. GFP Reporter Gene Signal is not Detectable Through the Egg Shell

Tail from GFP-expressing transgenic mice were incorporated into Chicken embryo (10 days) or placed outside of the shell. Only tail placed outside of the egg shell (FIG. 3A) can be observed with GFP fluorescence, whereas no signal is detected when placed inside the egg (FIG. 3B) Images were taken after 5 minutes thereafter using the Maestro 2.2 Imager (Cambridge Research & Instrumentation, Inc. USA).

FIG. 4. Detection of Female Avian Embryo

Luciferase reporter gene (star) is incorporated into the W chromosome of a female transgenic chicken (hen). Only female egg that carry the W and Z chromosomes, provide the reporter gene, specifically, luciferase signal.

FIG. 5. Detection of Male Avian Embryo

Luciferase reporter gene (star) is incorporated into the Z chromosomes of female transgenic chicken (hen). Male embryos are detected via the luciferase signal and females are free of foreign DNA.

### DETAILED DESCRIPTION OF THE INVENTION

Each day, billions of male chicks are being terminated via suffocation or grinding since they are not useful for laying eggs or to be bread for meat. The ability to determine the sex of the embryo before hatching is of high importance both ethically and financially. In the chicken- the genetic make-up of the sex chromosomes is ZZ for males and ZW for females. Meaning the W chromosome determines the gender of the female. This is unlike humans, in which it is the Y from the father that determines the male gender.

The invention provides a non-invasive efficient method for gender determination, using a reporter gene integrated in a gender specific chromosomes of transgenic avian subjects. Expression of this reporter gene in an embryo of an unhatched egg clearly and accurately identify the gender of said embryo.

Thus, a first aspect of the invention relates to a method of gender determination and optionally of selection of avian, or avian embryo in an unhatched egg, specifically, a fertilized unhatched egg. In some specific embodiments, the method may comprise the step of:

First, in step (a), providing or obtaining at least one transgenic avian subject or animal comprising at least one exogenous reporter gene integrated into at least one position or location in at least one of gender chromosome Z and W. In a second step (b) obtaining at least one fertilized egg from the transgenic avian subject, specifically animal or of any cells thereof.

The next step (c) involves determining in the egg if at least one detectable signal is detected. In more specific embodiments, detection of at least one detectable signal indicates the expression of the at least one reporter gene, thereby the presence of the W chromosome or Z chromosome in the avian embryo. Thus, in case the reporter gene has been integrated into the Z chromosome of a female transgenic avian, identification of a detectable signal in the examined egg indicate that the embryo has a maternal Z chromosome having a reporter gene integrated therein, and the embryo is thereby identified as male. Alternatively, in case the reporter gene has been integrated into the W chromosome of a female transgenic avian, identification of a detectable signal in the examined egg indicate that the embryo carries a maternal W chromosome and is therefore determined as female, thereby providing gender determination thereof.

It should be appreciated that the transgenic avian provided by the invention may be either a female or a male, as described in more detail herein after. In more specific embodiments, where the transgenic avian subject is a female, the egg identified by the method of the invention is laid by the transgenic female avian provided by the invention. In more specific embodiment, the transgenic female may be fertilized either by a transgenic male or by a wild type avian male. Still further, fertilization may occur either by mating or by insemination of the transgenic avian female with sperms obtained from a transgenic or wild type avian male. In yet other embodiments, where the transgenic avian is a male, egg identified by the method of the invention may be laid by either a wild type or transgenic female mated with the transgenic male provided by the invention, or inseminated by any cells thereof, specifically sperm cells that comprise the exogenous reporter gene of the invention integrated into the gender chromosomes thereof.

The invention thus provides a method for detecting a gender of an avian embryo within an unhatched fertilized egg. It should be appreciated that the method of the invention may be applicable for unhatched eggs of any embryonic stage of an avian embryo.

It should be noted that “Embryonic development stage or step of avian embryo”, as used herein refers to the stage of day 1 wherein the germinal disc is at the blastodermal stage and the segmentation cavity takes on the shape of a dark ring; the stage of day 2 wherein the first groove appears at the center of the blastoderm and the vitelline membrane appears; the stage of day 3 wherein blood circulation starts, the head and trunk can be discerned, as well as the brain and the cardiac structures which begins to beat; the stage of day 4 wherein the amniotic cavity is developing to surround the embryo and the allantoic vesicle appears; the stage of day 5 wherein the embryo takes a C shape and limbs are extending; the stage of day 6 wherein fingers of the upper and lower limbs becomes distinct; the stage of day 7 wherein the neck clearly separates the head from the body, the beak is formed and the brain progressively enters the cephalic region; the stage of day 8 wherein eye pigmentation is readily visible, the wings and legs are differentiated and the external auditory canal is opening; the stage of day 9 wherein claws appears and the first feather follicles are budding; the stage of day 10 wherein the nostrils are present, eyelids grow and the egg-tooth appears; the stage of day 11 wherein the palpebral aperture has an elliptic shape and the embryo has the aspect of a chick; the stage of day 12 wherein feather follicles surround the external auditory meatus and cover the upper eyelid whereas the lower eyelid covers major part of the cornea; the stage of day 13 wherein the allantois becomes the chorioallantoic membrane while claws and leg scales becomes apparent; the stage of days 14 to 16 wherein the whole body grows rapidly, vitellus shrinking accelerates and the egg white progressively disappears; the stage of day 17 wherein the renal system produces urates, the beak points to the air cell and the egg white is fully resorbed; the stage of day 18 wherein the vitellus internalized and the amount of amniotic fluid is reduced; the stage of day 19 wherein vitellus resorption accelerates and the beak is ready to pierce the inner shell membrane; the stage of day 20 wherein the vitellus is fully resorbed, the umbilicus is closed, the chick pierces the inner shell membrane, breathes in the air cell and is ready to hatch; the stage of day 21 wherein the chick pierces the shell in a circular way by means of its egg-tooth, extricates itself from the shell in 12 to 18 hours and lets its down dry off.

More specifically, the method of the invention may be applicable in determining the gender of an avian embryo in-ovo, inside the egg, at every stage of the embryonic developmental process. More specifically, from day 1, from day 2, from day 3, from day 4, from day 5, from day 6, from day 7, from day 8, from day 9, from day 10, from day 11, from day 12, from day 13, from day 14, from day 15, from day 16, from day 17, from day 18, from day 19, from day 20 and from day 21. More specifically, the method of the invention may be applicable for early detection of the embryo's gender, specifically, from day 1 to day 10, more specifically, between days 1 to 5.

As noted above, the method of the invention may be applicable for fertilized unhatched eggs. The term “fertilized egg” refers hereinafter to an egg laid by a hen wherein the hen has been mated by a rooster within two weeks, allowing deposit of male sperm into the female infundibulum and fertilization event to occur upon release of the ovum from the ovary. “Unhatched egg” as used herein, relates to an egg containing and embryo (also referred to herein as a fertile egg) within a structurally integral (not broken) shell.

The method of the invention is based on determination of a detectable signal formed by a reporter gene integrated into specific loci of the transgenic avian female or male laying the examined egg.

The “Integration of foreign or exogenous DNA/gene into chromosome” as used herein, refers hereinafter to a permanent modification of the nucleotide sequence of an organism chromosome. This modification is further transferred during cell division and if occurring in germinal cell lines, it will be transmitted also to offspring. In this case, the integrated reporter gene may be transferred to the embryo within the unhatched egg. The term “exogenous” as used herein, refers to originating from outside an organism that has been introduced into an organism for example by transformation or transfection with specifically manipulated vectors, viruses or any other vehicle. The integrated exogenous gene according to certain embodiments, may be a reporter gene. The term “reporter gene” relates to gene which encodes a polypeptide, whose expression can be detected in a variety of known assays and wherein the level of the detected signal indicates the presence of said reported.

As noted above, the exogenous reporter gene may be integrated into the avian gender chromosomes Z or W. The avian “gender chromosome Z or W” as used herein refers to the chromosomal system that determines the sex of offspring in chicken wherein males are the homogametic sex (ZZ), while females are the heterogametic sex (ZW). The presence of the W chromosome in the ovum determines the sex of the offspring while the Z chromosome is known to be larger and to possess more genes.

The method of the invention is based on the detection of a detectable signal that indicates and reflects the presence of the reporter gene and thereby the presence of a specific gender chromosome. “Detectable signal” refers hereinafter to a change in that is perceptible either by observation or instrumentally. Without limitations, the signal can be detected directly or only in the presence of a reagent. In some embodiments, detectable response is an optical signal including, but are not limited to chemiluminescent groups.

It should be appreciated that in some specific embodiments, at least one transgenic avian subject provided by the method of the invention, may comprise at least two different reporter genes, each reporter gene may be integrated into at least one position or location in one of gender chromosome Z or W. In case of at least two different reporter genes, each of the gender chromosomes may be labeled differently. The evaluation of the detectable signal formed, may indicate the gender of the examined embryo.

In yet some specific embodiments, the reporter gene comprised within the transgenic avian of the invention may be at least one bioluminescence reporter gene. Thus, in some embodiments the expressed polypeptide is a bioluminescence protein and accordingly the assay measures the levels of light emitted from bioluminescent reaction.

The term “bioluminescence” refers to the emission of light by biological molecules, such as proteins. Bioluminescence involves a molecular oxygen, an oxygenase, and a luciferase, which acts on a substrate, a luciferin, as will be described in more detail herein after.

In more specific embodiments, the reporter gene may be luciferase. The term “Luciferase” refers hereinafter to a class of oxidative enzymes that produce bioluminescence (photon emission). The emitted photon can be detected by light sensitive apparatus such as a luminometer or modified optical microscopes. Luciferase can be produced through genetic engineering in a variety of organisms mostly for use as a reporter gene. Luciferases occur naturally in bacteria, algae, fungi, jellyfish, insects, shrimp, and squid. In bacteria, the genes responsible for the light-emitting reaction (the lux genes encoded into the lux operon) have been isolated and used extensively in the construction of bio reporters that emit a blue-green light with a maximum intensity at 490 nm. Three variants of lux are available, one that functions at <30° C., another at <37° C., and a third at <45° C. The lux genetic system consists of five genes, luxA, luxB, luxC, luxD, and luxE. Depending on the combination of these genes used, several different types of bioluminescent bioreporters can be constructed. The luciferase protein is a heterodimer formed by the luxA and luxB gene products. The luxC, luxD, and luxE gene products encode for a reductase, transferase, and synthase respectively, that work together in a single complex to generate an aldehyde substrate for the bioluminescent reaction. luxAB bioreporters contain only the luxA and luxB genes, which are able to generate the light signal. However, to fully complete the light-emitting reaction, the substrate (long chain aldehyde) must be supplied to the cell.

On the other hand, luxCDABE bioreporters contain all five genes of the lux cassette, thereby allowing for a completely independent light generating system that requires no extraneous additions of substrate nor any excitation by an external light source. Due to their rapidity and ease of use, along with the ability to perform the bioassay repetitively in real time and on-line, makes luxCDABE bioreporters extremely attractive. Thus, in certain embodiments, the method of the invention may use as the reporter gene, the luxCDABE bioreporters.

In yet some further embodiments, the method of the invention may use as a reporter gene, the luc gene. Firefly luciferase (luc gene) catalyzes a reaction that produces visible light in the 550-575 nm range. A click-beetle luciferase is also available that produces light at a peak closer to 595 nm. Both luciferases require the addition of an exogenous substrate (luciferin) for the light reaction to occur.

It should be appreciated that any of the luciferases described herein, of any source known in the art, may be applicable for the methods and kits of the invention.

In yet some specific embodiments, the luciferase that may be used by the methods of the invention may be Gaussia princeps luciferase. In yet more specific embodiments, the luciferase used by the invention may be the luciferase encoded by the nucleic acid sequence as disclosed by GenBank: AY015993.1, having the amino acid sequence as disclosed by GenBank: AAG54095.1. In yet some further specific embodiments, the luciferase used by the methods and kits of the invention may be encoded by a nucleic acid sequence comprising the sequence as denoted by SEQ ID NO. 22. In yet some further embodiments, such luciferase may comprise the amino acid sequence as denoted by SEQ ID NO. 23, or any homologs, mutants or derivatives thereof.

In yet some further embodiments, luciferase used by the invention may be P. pyralis (firefly) luciferase. In some specific embodiments such luciferase may be the luciferase encoded by the nucleic acid sequence as disclosed by GenBank: M15077.1, having the amino acid sequence as disclosed by GenBank: AAA29795.1. In yet some further specific embodiments, the luciferase used by the methods and kits of the invention may be encoded by a nucleic acid sequence comprising the sequence as denoted by SEQ ID NO. 20. In yet some further embodiments, such luciferase may comprise the amino acid sequence as denoted by SEQ ID NO. 21, or any homologs, mutants or derivatives thereof.

As noted above, the luciferase used by the method of the invention may require supplementing additional reagents, specifically, a substrate.

Thus, in yet some further embodiments, the method may further comprise the step of providing to said egg of step (b), at least one of substrate and enzyme compatible to the bioluminescence reporter gene. It should be noted that such substrate or enzyme may be required for the formation of the detectable signal detected at step (c). More specifically, the method of the invention may comprise the step of providing to the egg of step (b), for example by injection, a substrate for luciferase. In some specific embodiments, such substrate may be luciferin. Luciferin, as used herein is a generic term for the light-emitting compound found in organisms that generate bioluminescence. Luciferins typically undergo an enzyme-catalyzed oxidation and the resulting excited state intermediate emits light upon decaying to its basal state. In yet some further embodiments, the substrate luciferin, that is an essential element in formation of said detectable signal, is injected to said egg, specifically, prior to measurement and determination of said signal, as performed in step (c). In some embodiments, the substrate may be injected at day 1, 2, 3, 4, 5, 6, 7, 8, 9, 10, 11, 12, 13, 14, 15, 16, 17, 18, 19, 20 or 21 of embryonal development of said avian subject, specifically animal. In yet some further embodiments, the substrate and/or further enzyme required for formation of the detectable signal may be provided to the fertilized egg as nucleic acid sequence encoding said substrate and/or enzyme, operably liked to said reporter gene. Such specific embodiments may refer for example to the use of the luxCDABE bioreporters as described above. LuxCDABE system contain five genes of the lux cassette, thereby allowing for a completely independent light generating system that requires no extraneous additions of substrate nor any excitation by an external light source.

In some embodiments, it should be noted that the detectable signal, specifically, the bioluminescent signal may be detected using suitable bioluminescent means. In some embodiments, the detectable signal formed by the luciferase reporter gene may be detected by light sensitive apparatus such as a luminometer or modified optical microscopes or Charge Coupled Device (CCD), a highly sensitive photon detector.

In still further embodiments, the at least one transgenic avian subject or animal provided by the method of the invention may be a female avian subject or animal. In more specific embodiments, where the at least one reporter gene is integrated into at least one position of female chromosome Z, detection of a detectable signal indicates that the embryo in the unhatched egg is male.

In yet some further embodiments, at least one transgenic avian subject or animal provided by the method of the invention may be a female avian subject or animal. In some specific embodiments, where the at least one reporter gene is integrated into at least one position of female chromosome W, detection of a detectable signal, indicates that the embryo in the unhatched egg is female.

In yet some further embodiments, the transgenic animal provided by the method of the invention may be a male subject having the reporter gene integrated into the Z chromosomes thereof. In such case, a detectable signal determined in an egg fertilized by such transgenic male or any sperms thereof, indicates that the embryo carries a paternal Z chromosome comprising the transgenic reporter gene, and is therefore male. In still further embodiments, detection of a detectable signal in an egg laid by a transgenic female avian fertilized by a transgenic male avian, both carrying the reporter gene of the invention integrated into the Z chromosomes thereof, may indicate in case of an intense signal that the embryo carries two copies of a reporter gene integrated into the female and male Z chromosomes thereof. In case of a less intense signal, the egg may be determined as a female.

As indicated herein before, the method of the invention involves the provision of transgenic avian animals. The preparation of transgenic avian animals, requires the use of genetic engineering approach that may use specific nucleases.

Thus, in yet more specific embodiments, the at least one reporter gene may be integrated into the gender chromosome of the transgenic avian subject or animal provided by the method of the invention using at least one programmable engineered nuclease (PEN). The term “programmable engineered nucleases (PEN)” as used herein, refers to synthetic enzymes that cut specific DNA sequences, derived from natural occurring nucleases involved in DNA repair of double strand DNA lesions and enabling direct genome editing.

The Clustered Regularly Interspaced Short Palindromic Repeats (CRISPR) Type II system is a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein.

As used herein, CRISPR arrays also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. Thus, in certain embodiments the CRISPR spacers used in the sequence encoding at least one gRNA of the methods and kits of the invention may comprise between 10 to 75 nucleotides (nt) each. More specifically, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 or more. In some specific embodiments the spacers comprise about 20 to 25 nucleotides, more specifically, about 20 nucleobases.

In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In more specific embodiments, PEN may be a clustered regularly interspaced short palindromic repeat (CRISPR) type II system.

More specifically, three major types of CRISPR-Cas system are delineated: Type I, Type II and Type III.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas 1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of cas9, cas1, cas2 csn2, and cas4 genes. It should be appreciated that any type II CRISPR-Cas systems may be applicable in the present invention, specifically, any one of type II-A or B.

Thus, in yet some further and alternative embodiments, the at least one cas gene used in the methods and kits of the invention may be at least one cas gene of type II CRISPR system (either typeII-A or typeII-B). In more particular embodiments, at least one cas gene of type II CRISPR system used by the methods and kits of the invention may be the cas9 gene. It should be appreciated that such system may further comprise at least one of cas1, cas2, csn2 and cas4 genes.

Double-stranded DNA (dsDNA) cleavage by Cas9 is a hallmark of “type II CRISPR-Gas” immune systems. The CRISPR-associated protein Cas9 is an RNA-guided DNA endonuclease that uses RNA:DNA complementarity to identify target sites for sequence-specific double stranded DNA (dsDNA) cleavage, creating the double strand brakes (DSBs) required for the HDR that results in the integration of the reporter gene into the specific target sequence, for example, a specific target within the avian gender chromosomes W and Z. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as proto-spacers. These proto-spacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

The target within the genome to be edited, specifically, the specific target loci within the gender chromosomes Z or W, where the reporter gene of the invention is to be integrated, should be present immediately upstream of a Protospacer Adjacent Motif (PAM).

The PAM sequence is absolutely necessary for target binding and the exact sequence is dependent upon the species of Cas9 (5′ NGG 3′ for Streptococcus pyogenes Cas9). In certain embodiments, Cas9 from S. pyogenes is used in the methods and kits of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. In some specific embodiments, the Cas9 of Streptococcus pyogenes M1 GAS, specifically, the Cas9 of protein id: AAK33936.1, may be applicable in the methods and kits of the invention. In some embodiments, the Cas9 protein may be encoded by the nucleic acid sequence as denoted by SEQ ID NO. 24. In further specific embodiments, the Cas9 protein may comprise the amino acid sequence as denoted by SEQ ID NO. 25, or any derivatives, mutants or variants thereof. Once expressed, the Cas9 protein and the gRNA, form a riboprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex binds any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA continues to anneal to the target DNA in a 3′ to 5′ direction.

Cas9 will only cleave the target if sufficient homology exists between the gRNA spacer and target sequences. Still further, the Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double strand break (DSB) within the target DNA that occurs about 3 to 4 nucleotides upstream of the PAM sequence.

The resulting DSB may be then repaired by one of two general repair pathways, the efficient but error-prone Non-Homologous End Joining (NHEJ) pathway and the less efficient but high-fidelity Homology Directed Repair (HDR) pathway. In some embodiments, the insertion that results in the specific integration of the reporter gene of the invention to the specific target loci within the gender chromosomes W or Z, is a result of repair of DSBs caused by Cas9. In some specific embodiments, the reporter gene of the invention is integrated, or knocked-in the target loci by HDR.

The term “Homology directed repair (HDR)”, as used herein refers to a mechanism in cells to repair double strand DNA lesions. The most common form of HDR is homologous recombination. The HDR repair mechanism can only be used by the cell when there is a homologue piece of DNA present in the nucleus, mostly in G2 and S phase of the cell cycle. When the homologue DNA piece is absent, another process called non-homologous end joining (NHEJ) can take place instead. Programmable engineered nucleases (PEN) strategies for genome editing, are based on cell activation of the HDR mechanism following specific double stranded DNA cleavage.

As discussed previously, Cas9 generates double strand breaks (DSBs) through the combined activity of two nuclease domains, RuvC and HNH. The exact amino acid residues within each nuclease domain that are critical for endonuclease activity are known (D10A for HNH and H840A for RuvC in S. pyogenes Cas9) and modified versions of the Cas9 enzyme containing only one active catalytic domain (called “Cas9 nickase”) have been generated. Cas9 nickases still bind DNA based on gRNA specificity, but nickases are only capable of cutting one of the DNA strands, resulting in a “nick”, or single strand break, instead of a DSB. DNA nicks are rapidly repaired by HDR (homology directed repair) using the intact complementary DNA strand as the template. Thus, two nickases targeting opposite strands are required to generate a DSB within the target DNA (often referred to as a “double nick” or “dual nickase” CRISPR system). This requirement dramatically increases target specificity, since it is unlikely that two off-target nicks will be generated within close enough proximity to cause a DSB. It should be therefore understood, that the invention further encompasses the use of the dual nickase approach to create a double nick-induced DSB for increasing specificity and reducing off-target effects.

Thus, in certain embodiments, the at least one reporter gene may be integrated into the gender chromosome of the transgenic avian subject, specifically animal by homology directed repair (HDR) mediated by at least one CRISPR/CRISPR-associated endonuclease 9 (Cas9) system.

In some further embodiments, the gRNA of the kit of the invention may comprise at least one CRISPR RNA (crRNA) and at least one trans-activating crRNA (tracrRNA).

In some alternative embodiments the kit of the invention may comprise nucleic acid sequence encoding the at least one gRNA. Such nucleic acid sequence may comprise a CRISPR array comprising at least one spacer sequence that targets and is therefore identical to at least one protospacer in a target genomic DNA sequence. It should be note that the nucleic acid sequence further comprises a sequence encoding at least one tracrRNA.

In some embodiments the CRISPR array according to the present disclosure comprises at least one spacer and at least one repeat. In yet another embodiment, the invention further encompasses the option of providing a pre-crRNA that can be processed to several final gRNA products that may target identical or different targets.

In yet some more specific embodiments, the crRNA comprised within the gRNA of the invention may be a single-stranded ribonucleic acid (ssRNA) sequence complementary to a target genomic DNA sequence. In some specific embodiments, the target genomic DNA sequence may be located immediately upstream of a protospacer adjacent motif (PAM) sequence and further.

As indicated herein, the gRNA of the kit of the invention may be complementary, at least in part, to the target genomic DNA. In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G).

As indicated above, the genomic DNA sequence targeted by the gRNA of the kit of the invention is located immediately upstream to a PAM sequence. In some embodiments, such PAM sequence may be of the nucleic acid sequence NGG.

In certain embodiments, the PAM sequence referred to by the invention may comprise N, that is any nucleotide, specifically, any one of Adenine (A), Guanine (G), Cytosine (C) or Thymine (T). In yet some further embodiments the PAM sequence according to the invention is composed of A, G, C, or T and two Guanines.

According to one embodiment, the polynucleotide encoding the gRNA of the invention may comprise at least one spacer and optionally, at least one repeat. In yet some further embodiments, the DNA encoding the gRNA of the invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers. In some embodiments, each spacer is located between two repeats. It should be further understood that the spacers of the nucleic acid sequence encoding the gRNA of the invention may be either identical or different spacers. In more embodiments, these spacers may target either an identical or different target genomic DNA. In yet some other embodiments, such spacer may target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target genomic DNA sequence. These target sequences may be derived from a single locus or alternatively, from several target loci.

As used herein, the term “spacer” refers to a non-repetitive spacer sequence that is designed to target a specific sequence and is located between multiple short direct repeats (i.e., CRISPR repeats) of CRISPR arrays. In some specific embodiments, spacers may comprise between about 15 to about 30 nucleotides, specifically, about 15, 16, 17, 18, 19, 20, 21, 22, 23, 24, 25, 26, 27, 28, 29, 30 or more nucleotides. More specifically, about 20-25 nucleotides.

The guide or targeting RNA encoded by the CRISPR system of the invention may comprise a CRISPR RNA (crRNA) and a trans activating RNA (tracrRNA). The sequence of the targeting RNA encoded by the CRISPR spacers is not particularly limited, other than by the requirement for it to be directed to (i.e., having a segment that is the same as or complementarity to) a target sequence in avian genomic DNA that is also referred to herein as a “proto-spacer”. Such proto-spacers comprise nucleic acid sequence having sufficient complementarity to a targeting RNA encoded by the CRISPR spacers comprised within the nucleic acid sequence encoding the gRNA of the methods and kits of the invention.

In some embodiments, a crRNA comprises or consists of 20, 21, 22, 23, 24, 25, 26, 27, 28, 29, 30, 31, 32, 33, 34, 35, 36, 37, 38, 39 or 40 nt of the spacer (targeting) sequence followed by 19-36 nt of repeat sequence. In specific and non-limiting embodiments, the targeting spacer may comprise or consist of a segment that targets any one of the genomic DNA sequence for which representative spacer sequences are indicated herein.

It should be noted that in some specific embodiments, the spacers of the CRISPR system of the invention may encode a targeting guide RNA (gRNA). A “gRNA” or “targeting RNA” is an RNA that, when transcribed from the portion of the CRISPR system encoding it, comprises at least one segment of RNA sequence that is identical to (with the exception of replacing T for U in the case of RNA) or complementary to (and thus “targets”) a DNA sequence in the target genomic DNA. The CRISPR systems of the present disclosure may optionally encode more than one targeting RNA, and the targeting RNAs be directed to one or more target sequences in the genomic DNA.

Still further, in some embodiments, the at least one reporter gene may be integrated into a gender chromosome of the transgenic avian subject, specifically animal by co-transfecting at least one cell of the avian subject, specifically animal or at least one cell introduced into the avian subject, specifically animal, with: (a) at least one first nucleic acid sequence comprising at least one nucleic acid sequence encoding at least one Cas9 protein and at least one nucleic acid sequence encoding at least one guide RNA (gRNA); and (b) at least one second nucleic acid sequence comprising at least one reporter gene.

Thus, for the preparation of a transgenic avian animal used by the methods of the invention, at least two nucleic acid molecules should be provided.

As used herein, “nucleic acids or nucleic acid molecules” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single-and double-stranded nucleic acids. As used herein, the term “nucleic acid(s)” also includes DNAs or RNAs as described above that contain one or more modified bases. As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 8 to about 1,000 nucleotides long. More specifically, the oligonucleotide molecule/s used by the kit of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200, 300, 400, 500, 600, 700, 800, 900, 1000 or more bases in length.

Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase).

In this connection an “isolated polynucleotide” is a nucleic acid molecule that is separated from the genome of an organism. For example, a DNA molecule that encodes the reporter gene used by the methods and kits of the invention or any derivatives or homologs thereof, as well as the sequences encoding the CRISPR/Cas9 and gRNAs of the methods and kits of the invention, that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species. In some embodiments, the nucleic acid sequences used by the methods and kits of the invention, specifically, nucleic acid sequences comprising sequences encoding the Cas9 and gRNA, or alternatively the reporter gene of the invention, may be provided constructed within a vector. The invention thus further relates to recombinant DNA constructs comprising the polynucleotides of the invention, and optionally, further additional elements such as promoters, regulatory and control elements, translation, expression and other signals, operably linked to the nucleic acid sequence of the invention.

As used herein, the terms “recombinant DNA”, “recombinant nucleic acid sequence” or “recombinant gene” refer to a nucleic acid comprising an open reading frame encoding one of the CRISPR system of the invention, specifically, the CRISPR/Cas9 type II, along with the gRNA of the invention that target the Cas9 to specific locus within avian chromosomes Z and/or W. In yet another embodiments, recombinant DNA as used herein further refers to a nucleic acid comprising an open reading frame encoding the reporter gene of the invention, specifically, transgene.

As referred to herein, by the term “gene” or “transgene” is meant a nucleic acid, either naturally occurring or synthetic, which encodes a protein product. The term “nucleic acid” is intended to mean natural and/or synthetic linear, circular and sequential arrays of nucleotides and nucleosides, e.g., cDNA, genomic DNA (gDNA), mRNA, and RNA, oligonucleotides, oligonucleosides, and derivatives thereof.

The phrase “operatively-linked” is intended to mean attached in a manner which allows for transgene transcription. The term “encoding” is intended to mean that the subject nucleic acid may be transcribed and translated into either the desired polypeptide or the subject protein in an appropriate expression system, e.g., when the subject nucleic acid is linked to appropriate control sequences such as promoter and enhancer elements in a suitable vector (e.g., an expression vector) and when the vector is introduced into an appropriate system or cell.

It should be appreciated that in some embodiments, at least one of the first and the second nucleic acid sequences provided and used by the methods and kits of the invention may be constructed and comprised within a vector. “Vectors” or “Vehicles”, as used herein, encompass vectors such as plasmids, phagemides, viruses, integratable DNA fragments, and other vehicles, which enable the integration of DNA fragments into the genome of the host, or alternatively, enable expression of genetic elements that are not integrated. Vectors are typically self-replicating DNA or RNA constructs containing the desired nucleic acid sequences, and operably linked genetic control elements that are recognized in a suitable host cell and effect the translation of the desired spacers. Generally, the genetic control elements can include a prokaryotic promoter system or a eukaryotic promoter expression control system. Such system typically includes a transcriptional promoter, transcription enhancers to elevate the level of RNA expression. Vectors usually contain an origin of replication that allows the vector to replicate independently of the host cell. In yet some alternative embodiments, the expression vectors used by the invention may comprise elements necessary for integration of the desired reporter gene of the invention into the avian gender specific chromosomes W and/or Z.

Accordingly, the term “control and regulatory elements” includes promoters, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding any desired protein using the method of this invention.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

To create the transgenic avian animal used by the methods of the invention, an avian cell comprising the reporter gene integrated into specific loci within the gender chromosomes Z or W thereof must be prepared. Such cell may be prepared by co-transfecting the cell with the first and second nucleic acid sequences provided by the methods and kits of the invention or with any construct comprising the same. “Transfection” as used herein is meant the process of inserting genetic material, such as DNA and double stranded RNA, into mammalian cells. The insertion of DNA into a cell enables the expression, or production, of proteins using the cells own machinery. Thus, co-transfection as used herein refers to simultaneous transfection of at least two different nucleic acid molecules or any vector comprising the same to each single cell. Still further, the nucleic acid sequences to be transfected can be transiently expressed for a short period of time, or become incorporated into the genomic DNA, where the change is passed on from cell to cell as it divides.

The invention therefore provides methods for an in-ovo gender determination of an avian embryo in-ovo based on expression of a reporter gene, specifically, luciferase. “Expression” generally refers to the process by which gene-encoded information is converted into the structures present and operating in the cell. Therefore, according to the invention “expression” of a reporter gene, specifically, may refer to transcription into a polynucleotide, translation into a protein, or even posttranslational modification of the protein.

In yet some further specific embodiments, the at least one reporter gene in the second nucleic acid sequence may be flanked at 5′ and 3′ thereof by homologous arms. It should be appreciated that in some embodiments, these arms are required and therefore facilitate HDR of the reporter gene at the integration site.

In more specific embodiments, the reporter gene in the second nucleic acid sequence used by the method of the invention, may be flanked with two arms that are homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to at least one nucleic acid sequence comprised within the target loci within the gender chromosomes Z or W, that serves as the integration site to facilitate specific integration via HDR. In certain embodiments, the target sequence is also referred to herein as at least one “proto-spacer” that is recognized by the “spacer” sequences that are part of the gRNA used by the invention, and provided by the first nucleic acid sequence.

The term “Homologous arms”, as used herein refers to HDR templates introduced into specific vectors or viruses, used to create specific mutations or insertion of new elements into a gene, that possess a certain amount of homology surrounding the target sequence to be modified (depending on which PEN is used). In yet some further specific embodiments, where CRISPR is used as a PEN, the arms sequences (left, upstream and right, downstream) may comprise between about 10 to 5000 bp, specifically, between about 50 to 1000 bp, between 100 to 500, specifically, 50, 100, 200, 300, 400, 500, 600, 700, 800, 900, 1000bp.

In yet some further embodiments, the targeting sequence within the gRNA encoded by the first nucleic acid sequence provided by the methods and kits of the invention, also referred to herein as the “spacer” sequence, exhibits homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to at least one nucleic acid sequence comprised within the target loci within the gender chromosomes Z or W, referred to herein as the “proto-spacer”.

In some embodiments, the at least one reporter gene in the second nucleic acid sequence may be operably linked to any one of a gender specific promoter, an embryonal specific promoter (for example α-Globin Promoter as referred in Mason et al. 1996) and an inducible promoter (for example light-inducible promoters derived from the soybean SSU gene claimed into U.S. Pat. No. 5,750,385, or derived from parsley chalcone synthase CHS promoter as referred in Weisshaar et al. 1991, or an engineered version of EL222, a bacterial Light-Oxygen-Voltage protein that activates expression when illuminated with blue light cited from Metta-Mena et al. 2014). In yet more specific embodiments, the reporter gene is under the control of an embryonic promoter, thereby limiting the expression of the transgenic reporter gene to the embryonal stage, with no expression in the adult chick. In such embodiment, the reporter transgene is used and expressed only at the embryonal stage, for diagnostic purposes.

More specifically, “Promoter” as used herein, refers to a particular region of the DNA that has the ability to control the expression of the gene which is placed downstream. Thus, “Promoter specific for gender in chicks” refers hereinafter to a promoter that will activate the expression of a gene, only in a specific chick gender (i.e. male or female). Still further, “Promoter specific for development in chicks” refers to a promoter that will activate the expression of a gene, only at specific stages of the chick development.

In some specific embodiments, the at least one reporter gene may be inserted and thereby integrated into at least one non-coding region of the target gender chromosome. Such approach avoids the disruption of genes that may be required for development and maturation of the unhatched embryo.

“Non-coding region” as used herein, refers to components of an organism's DNA that do not encode protein sequences. Some noncoding DNA region is transcribed into functional non-coding RNA molecules, other functions of noncoding DNA regions include the transcriptional and translational regulation of protein-coding sequences, scaffold attachment regions, origins of DNA replication, centromeres and telomeres. The hypothesized non-functional portion (or DNA of unknown function) has often been referred to as “junk DNA”.

In some specific embodiments, the at least one reporter gene may be integrated into at least one site at gender W chromosome. In more specific embodiments, the specific locus in the W chromosome may be location 1022859-1024215. In some specific embodiments, the target locus may comprise the nucleic acid sequence as denoted by SEQ ID NO. 3.

In more specific embodiments, the at least one gRNA required to target the reporter gene to such specific location within the W chromosome may comprises the nucleic acid sequence as denoted by any one of SEQ ID NO. 1 and 2, these gRNAs are designated herein as gRNA1 and gRNA2, respectively.

In yet some more specific embodiments, the gRNA used by the method of the invention to prepare the transgenic avian female may comprise the nucleic acid sequence as denoted by SEQ ID NO. 1 (gRNA1). In such case, the at least one reporter gene comprised within said second nucleic acid sequence may be flanked at 5′ and 3′ thereof by homologous arms comprising the amino acid sequence as denoted by SEQ ID NO. 4 and 5, that facilitate the integration thereof to said specific loci in W chromosome, respectively. It should be appreciated that these arms are also referred to herein as left and right arms, respectively.

In yet some alternative embodiments, the gRNA used for preparing the transgenic avian female of the invention may comprise the nucleic acid sequence as denoted by SEQ ID NO. 2 (gRNA2). In such case the at least one reporter gene comprised within the second nucleic acid sequence is flanked at 5′ and 3′ thereof by homologous arms comprising the amino acid sequence as denoted by SEQ ID NO. 6 and 7, respectively. It should be appreciated that these arms are also referred to herein as left and right arms, respectively.

In yet some further alternative embodiments, the at least one reporter gene used by the method of the invention for preparing the transgenic avian animal, may be integrated into at least one site at gender Z chromosome. In more specific embodiments, the specific loci in the Z chromosome may be any one of regions 9156874-9161874, as denoted by SEQ ID NO:15, 27764943-27769943, as denoted by SEQ ID NO:16, 42172748-42177748, as denoted by SEQ ID NO:17, 63363656-63368656, as denoted by SEQ ID NO:18 and 78777477-78782477, as denoted by SEQ ID NO:19 of Chromosome Z of female chicken.

In more specific embodiments, the at least one gRNA required to target the reporter gene to such specific location within the Z chromosome may comprises the nucleic acid sequence as denoted by any one of gRNA3: ACAGACCTATGATATGT, as denoted by SEQ ID NO. 11; gRNA4: CGATTATCACTCACAAG, as denoted by SEQ ID NO. 12; gRNA5: CTGGTTAGCATGGGGAC, as denoted by SEQ ID NO. 13; gRNA6: GTAAAGAGTCAGATACA, as denoted by SEQ ID NO. 14.

In yet some further embodiments, for integrating the reporter gene of the invention into the specific locus within the Z chromosome, left arm comprising the nucleic acid sequence as denoted by SEQ ID NO. 41 and right arm comprising the nucleic acid sequence as denoted by SEQ ID NO. 42 may be used to integrate the reporter gene of the invention to the specific loci directed by gRNA3 of SEQ ID NO: 11. In further embodiments, for integrating the reporter gene of the invention into the specific locus within the Z chromosome, left arm comprising the nucleic acid sequence as denoted by SEQ ID NO. 43, and right arm comprising the nucleic acid sequence as denoted by SEQ ID NO. 44, may be used to integrate the reporter gene of the invention to the specific loci directed by gRNA4 of SEQ ID NO:12. In still further embodiments, for integrating the reporter gene of the invention into the specific locus within the Z chromosome, left arm comprising the nucleic acid sequence as denoted by SEQ ID NO. 45, and right arm comprising the nucleic acid sequence as denoted by SEQ ID NO. 46, may be used to integrate the reporter gene of the invention to the specific loci directed by gRNA5 of SEQ ID NO:13. In some further embodiments, for integrating the reporter gene of the invention into the specific locus within the Z chromosome, left arm comprising the nucleic acid sequence as denoted by SEQ ID NO. 47, and right arm comprising the nucleic acid sequence as denoted by SEQ ID NO. 48, may be used to integrate the reporter gene of the invention to the specific loci directed by gRNA6 of SEQ ID NO:14. Further non-limiting examples for gRNA sequences suitable for integration into specific loci within the Z chromosome, may include but are not limited to gRNA7 of Z chromosome locus chrZ_42174515_-1, comprising the nucleic acid sequence GTAATACAGAGCTAAACCAG, as also denoted by SEQ ID NO:26, gRNA8 of Z chromosome locus chrZ_9157091_1, comprising the nucleic acid sequence ACAGACCTATGATATGTGAG, as also denoted by SEQ ID NO:27, gRNA9 of Z chromosome locus chrZ_27767602_-1, comprising the nucleic acid sequence GAGCTTGTGAGTGATAATCG, as also denoted by SEQ ID NO:28, gRNA10 of Z chromosome locus chrZ_78779927_1, comprising the nucleic acid sequence GTAAAGAGTCAGATACACAG, as also denoted by SEQ ID NO: 29, and gRNA11 of Z chromosome locus chrZ_63364946_-1, comprising the nucleic acid sequence CAGTGGGTACTGAAGCTGTG as also denoted by SEQ ID NO: 30.

In yet some further embodiments, for integrating the reporter gene of the invention into the specific locus within the Z chromosome, left arm comprising the nucleic acid sequence as denoted by SEQ ID NO. 31, and right arm comprising the nucleic acid sequence as denoted by SEQ ID NO. 32, may be used to integrate the reporter gene of the invention to the specific loci directed by gRNA7 of SEQ ID NO:26. In further embodiments, for integrating the reporter gene of the invention into the specific locus within the Z chromosome, left arm comprising the nucleic acid sequence as denoted by SEQ ID NO. 33, and right arm comprising the nucleic acid sequence as denoted by SEQ ID NO. 34, may be used to integrate the reporter gene of the invention to the specific loci directed by gRNA8 of SEQ ID NO:27. In still further embodiments, for integrating the reporter gene of the invention into the specific locus within the Z chromosome, left arm comprising the nucleic acid sequence as denoted by SEQ ID NO. 35, and right arm comprising the nucleic acid sequence as denoted by SEQ ID NO. 36, may be used to integrate the reporter gene of the invention to the specific loci directed by gRNA9 of SEQ ID NO:28. In further embodiments, for integrating the reporter gene of the invention into the specific locus within the Z chromosome, left arm comprising the nucleic acid sequence as denoted by SEQ ID NO. 37, and right arm comprising the nucleic acid sequence as denoted by SEQ ID NO. 38, may be used to integrate the reporter gene of the invention to the specific loci directed by gRNA10 of SEQ ID NO:29. In yet a further embodiment, for integrating the reporter gene of the invention into the specific locus within the Z chromosome, left arm comprising the nucleic acid sequence as denoted by SEQ ID NO. 39, and right arm comprising the nucleic acid sequence as denoted by SEQ ID NO. 40, may be used to integrate the reporter gene of the invention to the specific loci directed by gRNA11 of SEQ ID NO:30.

When genetic loci of zygote cells of an avian host, have been targeted and/or transfected with exogenous sequences, specifically, the reporter gene used by the invention, it may be desirable to use such cells to generate transgenic animals. For such a procedure, following the introduction of the targeting construct into the embryonic stem (ES) cells, the cells may be plated onto a feeder layer in an appropriate medium, for example, DMEM supplemented with growth factors and cytokines, fetal bovine serum and antibiotics. The embryonic stem cells may have a single targeted locus (heterozygotic) or both loci targeted (homozygotic). Cells containing the construct may be detected by employing a selective medium and after sufficient time for colonies to grow, colonies may be picked and analyzed for the occurrence of gene targeting. In some specific embodiments, PCR may be applied to verify the integration of the desired exogenous sequences into the target loci, using primers within and outside the construct sequence. Colonies which show gene targeting may then be used for injection into avian embryos. The ES cells can then be trypsinized and the modified cells can be injected through an opening made in the side of the egg. After sealing the eggs, the eggs can be incubated under appropriate conditions until hatching. Newly hatched avian can be tested for the presence of the target construct sequences, for example by examining a biological sample thereof, e.g., a blood sample. After the avian have reached maturity, they are bred and their progeny may be examined to determine whether the exogenous integrated sequences are transmitted through the germ line.

Chimeric avian are generated which are derived in part from the modified embryonic stem cells or zygote cells, capable of transmitting the genetic modifications through the germ line. Mating avian strains containing exogenous sequences, specifically, the reporter gene used by the invention, or portions thereof, with strains in which the avian wild type loci, or portions thereof, is restored, should result in progenies displaying an in-ovo detectable gender.

Still further, transgenic avian can also be produced by other methods, some of which are discussed below. Among the avian cells suitable for transformation for generating transgenic animals are primordial germ cells (PGC), sperm cells and zygote cells (including embryonic stem cells). Sperm cells can be transformed with DNA constructs by any suitable method, including electroporation, microparticle bombardment, lipofection and the like. The sperm can be used for artificial insemination of avian. Progeny of the inseminated avian can be examined for the exogenous sequence as described above.

Alternatively, primordial germ cells may be isolated from avian eggs, transfected with the exogenous reporter gene of the invention by any appropriate method, and transferred or inserted into new embryos, where they can become incorporated into the developing gonads. Hatched avian and their progeny can be examined for the exogenous reporter gene sequence as described by the invention.

In yet another approach, dispersed blastodermal cells isolated from eggs can be transfected by any appropriate means with the exogenous reporter gene sequence, or portions thereof, integrated to the gender specific chromosomes Z or W, followed by injection into the subgerminal cavity of intact eggs. Hatched avian subjects and their progeny may be examined for the exogenous reporter gene as described above.

Chicken primordial germ cells (PGCs) are the precursors for ova and spermatozoa. Thus, in some aspects thereof, the invention provides the production of transgenic chickens via a germline transmission system using PGCs co-transfected with the reporter gene construct and with the CRISPR/Cas9 gRNA construct that directs the integration of the reporter gene into the gender specific chromosomes W and Z. PGCs are sorted and transferred into the bloodstream of 2.5-day recipient embryos for germline transmission.

Thus, in some specific embodiments, the “Preparation of transgenic avian animal” refers to a multi-step method involving genetic engineering techniques for production of chicken with genomic modifications wherein a) Primordial Germ Cells (PGCs) are isolated from the blood of two days-old chick embryos; b) a transgene construct is incorporated into cultured PGCs by using lentiviral system, Piggybac transposon vectors, TALENS or CRISPR/Cas9 techniques; (c) transgenic PGCs are identified and injected into the circulatory system of embryos and migrate to the developing gonads; d) recipient embryos are incubated at 37° C. until hatching (d) hatched males are reared to sexual maturity and crossed with wild-type hens (e) offspring are screened to identify those derived from the transgenic PGCs.

Thus, in a second aspect, the invention relates to an avian transgenic animal comprising, in at least one cell thereof, at least one exogenous reporter gene integrated into at least one position or location (also referred to herein as locus) in at least one of gender chromosome Z and W.

The term “avian” relates to any species derived from birds characterized by feathers, toothless beaked jaws, the laying of hard-shelled eggs, a high metabolic rate, a four-chambered heart, and a lightweight but strong skeleton. Avian species includes, without limitation, chicken, quail, turkey, duck, Gallinacea sp, goose, pheasant and other fowl. The term “hen” includes all females of the avian species. A “transgenic avian” generally refers to an avian that has had a heterologous DNA sequence, or one or more additional DNA sequences normally endogenous to the avian (collectively referred to herein as “transgenes”) chromosomally integrated into the germ cells of the avian. As a result of such transfer and integration, the transferred sequence may be transmitted through germ cells to the offspring of a transgenic avian. The transgenic avian (including its progeny) also have the transgene integrated into the gender chromosomes of somatic cells.

In some specific embodiments, the at least one transgenic animal of the invention may comprise at least two different reporter genes. In such case, each reporter gene may be integrated into at least one position or location in one of gender chromosome Z or W.

In yet some further embodiments, the reporter gene comprised within the transgenic animal of the invention, may be at least one bioluminescence reporter gene.

In more specific embodiments, such bioluminescence reporter gene may comprise or may be luciferase.

In certain embodiments, the at least one transgenic avian animal provided by the invention, may be female. In more specific embodiments, the at least one reporter gene in such transgenic avian female may be integrated into at least one position of the female chromosome Z.

In yet some alternative embodiments, the at least one transgenic avian animal may be female, having at least one reporter gene integrated into at least one position of the female chromosome W.

In some specific embodiments, the at least one reporter gene may be integrated into the gender chromosome of the transgenic animal of the invention using at least one PEN.

More specifically, such PEN may be in certain embodiments, a CRISPR type II system.

In yet more specific embodiments, the at least one reporter gene may be integrated into the gender chromosome of the transgenic avian animal of the invention by HDR mediated by at least one CRISPR/Cas9 system.

In more specific embodiments, the at least one reporter gene may be integrated into a gender chromosome of the transgenic avian animal of the invention by co-transfecting at least one cell of this avian animal, or at least one cell that is to be introduced into said avian animal with at least two nucleic acid sequences. More specifically, such cell may be co-transfected with (a) at least one first nucleic acid sequence comprising at least one nucleic acid sequence encoding at least one Cas9 protein and at least one nucleic acid sequence encoding at least one gRNA, thereby providing a CRISPR mediated integration; and (b) at least one second nucleic acid sequence comprising at least one reporter gene.

In more specific embodiments, the at least one reporter gene in the second nucleic acid sequence may be flanked at 5′ and 3′ thereof by homologous arms. These arms exhibit homology to the integration target site within the target gender chromosome, thereby facilitating HDR at the integration site.

In yet more specific embodiments, the at least one reporter gene in the second nucleic acid sequence may be operably linked to any one of a gender specific promoter, an embryonal specific promoter and an inducible promoter. Such promoter should limit the expression of the reporter gene of the invention to the specific desired gender (in case of gender specific promoter), the specific embryonic stage (embryonic specific promoter) or specific conditions (inducible conditions).

In yet some further specific embodiments, the at least one reporter gene comprised within the transgenic avian animal of the invention may be integrated into at least one non-coding region of one of its gender chromosomes.

In certain embodiments, the at least one reporter gene may be integrated into at least one site at gender W chromosome. In some particular embodiments, the integration site may be located at locus 1022859-1024215 at the W chromosome, specifically, galGal5_dna range of chromosome W:1022859-1024215. In yet some further specific embodiments, such loci comprises the nucleic acid sequence as denoted by SEQ ID NO. 3.

For specific integration of the reporter gene of the invention at any position within the loci described above, specific gRNAs may be required. Therefore, in some particular and non-limiting embodiments, appropriate gRNAs used for the preparation of the transgenic avian animal of the invention may comprise the nucleic acid sequence as denoted by any one of SEQ ID NO. 1 and 2. In some specific embodiments, these gRNAs are referred to herein as gRNA1 and gRNA2, respectively.

In some particular embodiments, the transgenic avian animal provided by the invention has been prepared using a gRNA1 that comprises the nucleic acid sequence as denoted by SEQ ID NO. 1. To enable integration of the reporter gene of the invention in such specific location, the reporter gene that should be integrated, must carry in certain embodiments, particular arms facilitating incorporation thereof in the target integration site directed by the gRNA used. Thus, in some specific embodiments, the at least one reporter gene may be comprised within the second nucleic acid sequence, where this reporter gene is flanked at 5′ and 3′ thereof by homologous arms comprising the amino acid sequence as denoted by SEQ ID NO. 4 and 5, respectively.

In yet some alternative embodiments, the transgenic avian animal provided by the invention may be prepared using a gRNA2 that comprises the nucleic acid sequence as denoted by SEQ ID NO. 2. In such case, to enable integration of the reporter gene of the invention at the specific site recognized by said gRNA2, the at least one reporter gene comprised within the second nucleic acid sequence may be according to specific embodiments, flanked at 5′ and 3′ thereof by homologous arms comprising the amino acid sequence as denoted by SEQ ID NO. 6 and 7, respectively.

In yet some further alternative embodiments, the transgenic avian animal of the invention may comprise at least one reporter gene integrated into at least one site at gender Z chromosome. In some particular and non-limiting embodiments, such avian transgenic animal may be female that carry the transgenic reporter gene integrated into the Z chromosome. In more specific embodiments, the specific loci in the Z chromosome may be any one of regions 9156874-9161874, as denoted by SEQ ID NO:15, 27764943-27769943, as denoted by SEQ ID NO:16, 42172748-42177748, as denoted by SEQ ID NO:17, 63363656-63368656, as denoted by SEQ ID NO:18 and 78777477-78782477, as denoted by SEQ ID NO:19 of Chromosome Z of female chicken.

In more specific embodiments, the at least one gRNA required to target the reporter gene to such specific location within the Z chromosome may comprises the nucleic acid sequence as denoted by any one of gRNA3: ACAGACCTATGATATGT, as denoted by SEQ ID NO. 11; gRNA4: CGATTATCACTCACAAG, as denoted by SEQ ID NO. 12; gRNA5: CTGGTTAGCATGGGGAC, as denoted by SEQ ID NO. 13 ; gRNA6: GTAAAGAGTCAGATACA, as denoted by SEQ ID NO. 14.

In yet some further embodiments, for integrating the reporter gene of the invention into the specific locus within the Z chromosome, left arm comprising the nucleic acid sequence as denoted by SEQ ID NO. 41, and right arm comprising the nucleic acid sequence as denoted by SEQ ID NO. 42, may be used to integrate the reporter gene of the invention to the specific loci directed by gRNA3 of SEQ ID NO:11. In further embodiments, for integrating the reporter gene of the invention into the specific locus within the Z chromosome, left arm comprising the nucleic acid sequence as denoted by SEQ ID NO. 43, and right arm comprising the nucleic acid sequence as denoted by SEQ ID NO. 44, may be used to integrate the reporter gene of the invention to the specific loci directed by gRNA4 of SEQ ID NO:12. In still further embodiments, for integrating the reporter gene of the invention into the specific locus within the Z chromosome, left arm comprising the nucleic acid sequence as denoted by SEQ ID NO. 45, and right arm comprising the nucleic acid sequence as denoted by SEQ ID NO. 46, may be used to integrate the reporter gene of the invention to the specific loci directed by gRNA5 of SEQ ID NO:13. In some further embodiments, for integrating the reporter gene of the invention into the specific locus within the Z chromosome, left arm comprising the nucleic acid sequence as denoted by SEQ ID NO. 47, and right arm comprising the nucleic acid sequence as denoted by SEQ ID NO. 48, may be used to integrate the reporter gene of the invention to the specific loci directed by gRNA6 of SEQ ID NO:14.

Further non-limiting examples for gRNA sequences suitable for integration into specific loci within the Z chromosome, may include but are not limited to gRNA7 of Z chromosome locus chrZ_42174515_-1, comprising the nucleic acid sequence GTAATACAGAGCTAAACCAG, as also denoted by SEQ ID NO:26, gRNA8 of Z chromosome locus chrZ_9157091_1, comprising the nucleic acid sequence ACAGACCTATGATATGTGAG, as also denoted by SEQ ID NO:27, gRNA9 of Z chromosome locus chrZ_27767602_-1, comprising the nucleic acid sequence GAGCTTGTGAGTGATAATCG, as also denoted by SEQ ID NO:28, gRNA10 of Z chromosome locus chrZ_78779927_1, comprising the nucleic acid sequence GTAAAGAGTCAGATACACAG, as also denoted by SEQ ID NO: 29, and gRNA11 of Z chromosome locus chrZ_63364946_-1, comprising the nucleic acid sequence CAGTGGGTACTGAAGCTGTG as also denoted by SEQ ID NO: 30.

In yet some further embodiments, for integrating the reporter gene of the invention into the specific locus within the Z chromosome, left arm comprising the nucleic acid sequence as denoted by SEQ ID NO. 31, and right arm comprising the nucleic acid sequence as denoted by SEQ ID NO. 32, may be used to integrate the reporter gene of the invention to the specific loci directed by gRNA7 of SEQ ID NO:26. In further embodiments, for integrating the reporter gene of the invention into the specific locus within the Z chromosome, left arm comprising the nucleic acid sequence as denoted by SEQ ID NO. 33, and right arm comprising the nucleic acid sequence as denoted by SEQ ID NO. 34, may be used to integrate the reporter gene of the invention to the specific loci directed by gRNA8 of SEQ ID NO:27. In still further embodiments, for integrating the reporter gene of the invention into the specific locus within the Z chromosome, left arm comprising the nucleic acid sequence as denoted by SEQ ID NO. 35, and right arm comprising the nucleic acid sequence as denoted by SEQ ID NO. 36, may be used to integrate the reporter gene of the invention to the specific loci directed by gRNA9 of SEQ ID NO:28. In further embodiments, for integrating the reporter gene of the invention into the specific locus within the Z chromosome, left arm comprising the nucleic acid sequence as denoted by SEQ ID NO. 37, and right arm comprising the nucleic acid sequence as denoted by SEQ ID NO. 38, may be used to integrate the reporter gene of the invention to the specific loci directed by gRNA10 of SEQ ID NO:29. In yet a further embodiment, for integrating the reporter gene of the invention into the specific locus within the Z chromosome, left arm comprising the nucleic acid sequence as denoted by SEQ ID NO. 39, and right arm comprising the nucleic acid sequence as denoted by SEQ ID NO. 40, may be used to integrate the reporter gene of the invention to the specific loci directed by gRNA11 of SEQ ID NO:30.

In yet another aspect, the invention relates to a cell comprising at least one exogenous reporter gene integrated into at least one position or location in at least one of gender chromosome Z and W.

In some specific embodiments, the cell provided by the invention may be an avian cell.

In some particular embodiments, the avian cell provided by the invention may be a primordial germ cell (PGC).

The term “germ cells” refers to an embryonic cell that upon uniting with another germ cells develops into a gamete. “Primordial germ cells (PGCs)”, as used herein relates to germline stem cells that serve as progenitors of the gametes and give rise to pluripotent embryonic stem cells. The cells in the gastrulating embryo that are signaled to become PGCs during embryogenesis, migrate into the genital ridges which becomes the gonads, and differentiate into mature gametes.

In some specific embodiments, the at least one cell of the invention may comprise at least two different reporter genes. In such case, each reporter gene may be integrated into at least one position or location in one of gender chromosome Z or W.

In yet some further embodiments, the reporter gene comprised within the transgenic animal of the invention, may be at least one bioluminescence reporter gene. In more specific embodiments, such bioluminescence reporter gene may comprise or may be luciferase.

In certain embodiments, the at least one transgenic cell provided by the invention, may comprise the at least one reporter gene integrated into at least one position of at least one chromosome Z thereof. It should be noted that in some embodiments such cell may be either a female avian cell or a male avian cell.

In yet some alternative embodiments, the at least one transgenic cell of the invention, may carry at least one reporter gene integrated into at least one position of chromosome W. It should be noted that in some embodiments such cell may be a female avian cell.

In some specific embodiments, the at least one reporter gene may be integrated into the gender chromosome of the transgenic cell of the invention using at least one PEN.

More specifically, such PEN may be in certain embodiments, a CRISPR type II system.

In yet more specific embodiments, the at least one reporter gene may be integrated into the gender chromosome of the transgenic cell of the invention by HDR mediated by at least one CRISPR/Cas9 system.

In yet some further embodiments, the cell provided by the invention may comprise at least one reporter gene integrated into a gender chromosome of the cell. In more specific embodiments, such specific integration of the reporter gene may be enabled by co-transfecting the cell with: (a) at least one first nucleic acid sequence comprising at least one nucleic acid sequence encoding at least one Cas9 protein and at least one nucleic acid sequence encoding at least one guide RNA (gRNA); and (b) at least one second nucleic acid sequence comprising at least one said reporter gene.

In certain embodiments, the at least one reporter gene in the second nucleic acid sequence co-transfected to the cell of the invention, may be flanked at 5′ and 3′ thereof by homologous arms for HDR at the integration site.

In yet more specific embodiments, the at least one reporter gene in the second nucleic acid sequence may be operably linked to any one of a gender specific promoter, an embryonal specific promoter and an inducible promoter. Such promoter should limit the expression of the reporter gene of the invention to the specific desired gender (in case of gender specific promoter), the specific embryonic stage (embryonic specific promoter) or specific conditions (inducible conditions).

In yet some further specific embodiments, the at least one reporter gene comprised within the transgenic cell of the invention may be integrated into at least one non-coding region of one of its gender chromosomes.

In certain embodiments, the at least one reporter gene may be integrated into at least one site at gender W chromosome. In some particular embodiments, the integration site may be located at locus 1022859-1024215 at the W chromosome, specifically, galGal5_dna range of chromosome W:1022859-1024215. In yet some further specific embodiments, such loci comprises the nucleic acid sequence as denoted by SEQ ID NO. 3.

For specific integration of the reporter gene of the invention at any position within the loci described above, specific gRNAs may be required. Therefore, in some particular and non-limiting embodiments, appropriate gRNAs used for the preparation of the transgenic avian animal of the invention may comprise the nucleic acid sequence as denoted by any one of SEQ ID NO. 1 and 2. In some specific embodiments, these gRNAs are referred to herein as gRNA1 and gRNA2, respectively.

In some particular embodiments, to target the integration of the reporter gene to chromosome W in the cell provided by the invention, specific gRNAs should be used. In further particular embodiments, the gRNA may comprise the nucleic acid sequence as denoted by SEQ ID NO. 1 referred to herein as gRNA1. In such case, the at least one reporter gene comprised within the second nucleic acid sequence, may be flanked at 5′ and 3′ thereof by homologous arms comprising the amino acid sequence as denoted by SEQ ID NO. 4 and 5, respectively.

In yet some further alternative embodiments, the cell provided by the invention may be prepared by using gRNA referred to herein as gRNA2. In certain embodiments, gRNA2 may comprise the nucleic acid sequence as denoted by SEQ ID NO. 2. In such specific embodiments, the at least one reporter gene comprised within the second nucleic acid sequence may be flanked at 5′ and 3′ thereof by homologous arms comprising the amino acid sequence as denoted by SEQ ID NO. 6 and 7, respectively.

In yet some further alternative embodiments, the cell provided by the invention may be prepared by integrating the at least one reporter gene of the invention into the Z chromosome of the cell. In certain embodiments, for preparing the cell of the invention, the at least one reporter gene may be integrated into at least one site at gender Z chromosome. In more specific embodiments, the specific loci in the Z chromosome may be any one of regions 9156874-9161874, as denoted by SEQ ID NO:15, 27764943-27769943, as denoted by SEQ ID NO:16, 42172748-42177748, as denoted by SEQ ID NO:17, 63363656-63368656, as denoted by SEQ ID NO:18 and 78777477-78782477, as denoted by SEQ ID NO:19 of Chromosome Z of female chicken.

In more specific embodiments, the at least one gRNA required to target the reporter gene to such specific location within the Z chromosome of the cell of the invention may comprises the nucleic acid sequence as denoted by any one of gRNA3: ACAGACCTATGATATGT, as denoted by SEQ ID NO. 11; gRNA4: CGATTATCACTCACAAG, as denoted by SEQ ID NO. 12; gRNA5: CTGGTTAGCATGGGGAC, as denoted by SEQ ID NO. 13; gRNA6: GTAAAGAGTCAGATACA, as denoted by SEQ ID NO. 14.

In yet some further embodiments, for integrating the reporter gene of the invention into the specific locus within the Z chromosome, left arm comprising the nucleic acid sequence as denoted by SEQ ID NO. 41, and right arm comprising the nucleic acid sequence as denoted by SEQ ID NO. 42, may be used to integrate the reporter gene of the invention to the specific loci directed by gRNA3 of SEQ ID NO:11. In further embodiments, for integrating the reporter gene of the invention into the specific locus within the Z chromosome, left arm comprising the nucleic acid sequence as denoted by SEQ ID NO. 43, and right arm comprising the nucleic acid sequence as denoted by SEQ ID NO. 44, may be used to integrate the reporter gene of the invention to the specific loci directed by gRNA4 of SEQ ID NO:12. In still further embodiments, for integrating the reporter gene of the invention into the specific locus within the Z chromosome, left arm comprising the nucleic acid sequence as denoted by SEQ ID NO. 45, and right arm comprising the nucleic acid sequence as denoted by SEQ ID NO. 46, may be used to integrate the reporter gene of the invention to the specific loci directed by gRNA5 of SEQ ID NO:13. In some further embodiments, for integrating the reporter gene of the invention into the specific locus within the Z chromosome, left arm comprising the nucleic acid sequence as denoted by SEQ ID NO. 47, and right arm comprising the nucleic acid sequence as denoted by SEQ ID NO. 48, may be used to integrate the reporter gene of the invention to the specific loci directed by gRNA6 of SEQ ID NO:14. Further non-limiting examples for gRNA sequences suitable for integration into specific loci within the Z chromosome, may include but are not limited to gRNA7 of Z chromosome locus chrZ_42174515_-1, comprising the nucleic acid sequence GTAATACAGAGCTAAACCAG, as also denoted by SEQ ID NO:26, gRNA8 of Z chromosome locus chrZ_9157091_1, comprising the nucleic acid sequence ACAGACCTATGATATGTGAG, as also denoted by SEQ ID NO:27, gRNA9 of Z chromosome locus chrZ_27767602_-1, comprising the nucleic acid sequence GAGCTTGTGAGTGATAATCG, as also denoted by SEQ ID NO:28, gRNA10 of Z chromosome locus chrZ_78779927_1, comprising the nucleic acid sequence GTAAAGAGTCAGATACACAG, as also denoted by SEQ ID NO: 29, and gRNA11 of Z chromosome locus chrZ_63364946_-1, comprising the nucleic acid sequence CAGTGGGTACTGAAGCTGTG as also denoted by SEQ ID NO: 30.

In yet some further embodiments, for integrating the reporter gene of the invention into the specific locus within the Z chromosome, of the cell of the invention, left arm comprising the nucleic acid sequence as denoted by SEQ ID NO. 31, and right arm comprising the nucleic acid sequence as denoted by SEQ ID NO. 32, may be used to integrate the reporter gene of the invention to the specific loci directed by gRNA7 of SEQ ID NO:26. In further embodiments, for integrating the reporter gene of the invention into the specific locus within the Z chromosome, left arm comprising the nucleic acid sequence as denoted by SEQ ID NO. 33, and right arm comprising the nucleic acid sequence as denoted by SEQ ID NO. 34, may be used to integrate the reporter gene of the invention to the specific loci directed by gRNA8 of SEQ ID NO:27. In still further embodiments, for integrating the reporter gene of the invention into the specific locus within the Z chromosome, left arm comprising the nucleic acid sequence as denoted by SEQ ID NO. 35, and right arm comprising the nucleic acid sequence as denoted by SEQ ID NO. 36, may be used to integrate the reporter gene of the invention to the specific loci directed by gRNA9 of SEQ ID NO:28. In further embodiments, for integrating the reporter gene of the invention into the specific locus within the Z chromosome, left arm comprising the nucleic acid sequence as denoted by SEQ ID NO. 37, and right arm comprising the nucleic acid sequence as denoted by SEQ ID NO. 38, may be used to integrate the reporter gene of the invention to the specific loci directed by gRNA10 of SEQ ID NO:29. In yet a further embodiment, for integrating the reporter gene of the invention into the specific locus within the Z chromosome, left arm comprising the nucleic acid sequence as denoted by SEQ ID NO. 39, and right arm comprising the nucleic acid sequence as denoted by SEQ ID NO. 40, may be used to integrate the reporter gene of the invention to the specific loci directed by gRNA11 of SEQ ID NO:30.

In yet some further aspects thereof, the invention encompasses any egg derived, laid or fertilized by at least one of any of the transgenic avian subjects or animals of the invention, or by any progeny thereof, any component or any parts thereof or any product comprising said egg, components or parts thereof. It should be understood that in some embodiments, such transgenic avian subjects may comprise, in at least one cell thereof, at least one exogenous reporter gene integrated into at least one position or location (also referred to herein as locus) in at least one of gender chromosome Z and W.

The term “egg” as used herein, encompasses fertilized as well as non-fertilized eggs. More specifically, a fertilized egg is the organic vessel containing the zygote (that results from fertilization of an ovum), in which an avian embryo develops, at which point the animal hatches. A zygote is a eukaryotic cell formed by a fertilization event between two gametes, specifically, the ovum and the sperm. A non-fertilized egg comprises the ovum, that is the egg cell (plural ova), that forms the female gamete (reproductive cell) in oogamous organisms.

At lay, a typical egg weighs around 55 to 60 g and consists of three main components (also referred to herein as parts): eggshell (9-12%), egg white (60%), and yolk (30-33%). Whole egg is composed of water (75%), proteins (12%), lipids (12%), and carbohydrates and minerals (1). Proteins present in egg are distributed among the egg white and yolk, whereas lipids are mainly concentrated in the yolk. Yolk is covered with the vitelline membrane and mainly consists of water (50%), protein (15-17%), lipids (31-35%), and carbohydrates (1%). Protein present in egg yolk consists of lipovitellins (36%), livetins (38%), phosvitin (8%), and low-density lipoproteins (17%). Also, yolk contains 1% carotinoides, which makes it yellow in color. Egg white mainly consists of water (88%) and protein (11%), with the remainder consisting of carbohydrates, ash, and trace amounts of lipids (1%). Ovalbumin (54%), ovotransferrin (12%), ovomucoid (11%), lysozyme (3.5%), and ovomucin (3.5%) are considered as the main proteins and avidin (0.05%), cystatin (0.05%), ovomacroglobulin (0.5%), ovoflavoprotein (0.8%), ovoglycoprotein (1.0%), and ovoinhibitor (1.5%) are the minor proteins found in egg white.

It should be understood that in some embodiments any of the egg parts, components, or proteins, specifically, any of the parts, elements or components disclosed herein are part of the invention. It should be appreciated that the egg/s of the invention may be laid by any of the transgenic avian provided herein or by any progenies thereof. Such transgenic avian subject may be either a female or a male. In more specific embodiments, where the transgenic avian subject is a female, the egg/s of the invention may be laid in some embodiments by the transgenic female avian provided by the invention. In more specific embodiment, in case the egg/s of the invention is a fertilized egg, the transgenic female may be fertilized either by a transgenic male or by a wild type avian male. Still further, fertilization may occur either by mating or by insemination of the transgenic avian female with sperms obtained from a transgenic or wild type avian male. In yet other embodiments, where the transgenic avian is a male, the egg/s provided by the invention may be laid by either a wild type or transgenic female mated with the transgenic male provided by the invention, or inseminated by any cells thereof, specifically sperm cells that comprise the exogenous reporter gene of the invention integrated into the gender chromosomes thereof.

As indicated above, the egg/s of the invention may be any egg/s laid or fertilized by the transgenic avian subjects provided by the invention. In some embodiments, the egg/s may be a fertilized egg. In yet some further embodiments, the fertilized egg may contain the reporter gene of the invention integrated into at least one gender chromosomes thereof.

In some specific embodiments, the egg/s of the invention may be laid or fertilized by at least one transgenic animal of the invention that may comprise at least two different reporter genes. In such case, each reporter gene may be integrated into at least one position or location in one of gender chromosome Z or W.

In yet some further embodiments, the reporter gene comprised within the transgenic animal that either laid or fertilized the egg of the invention, may be at least one bioluminescence reporter gene. In more specific embodiments, such bioluminescence reporter gene may comprise or may be luciferase.

In certain embodiments, the at least one transgenic avian animal laid or fertilized the egg of the invention, may be female. In more specific embodiments, the at least one reporter gene in such transgenic avian female may be integrated into at least one position of the female chromosome Z. In some embodiments, a fertilized egg laid by such transgenic female avian subject may according to some embodiments carry the reporter gene integrated to its Z chromosome and as such, may carry a male embryo. In some embodiments, such egg may be referred to herein as a labeled egg or as a transgenic egg. In yet some alternative embodiment, such fertilized egg may carry a paternal unlabeled Z chromosome and a maternal unlabeled W chromosome, and therefore may carry an unlabeled female embryo. In some embodiments, such egg may be referred to herein as an unlabeled egg or as a non-transgenic egg (or WT or normal egg).

In yet some alternative embodiments, the at least one transgenic avian animal laid or fertilized the egg/s of the invention may be female, having at least one reporter gene integrated into at least one position of the female chromosome W. In such case, a fertilized egg laid by such transgenic female avian subject may according to some embodiments carry the reporter gene integrated to its W chromosome and as such, may carry a labeled female embryo. In some embodiments, such egg may be referred to herein as a labeled egg or as a transgenic egg. In yet some alternative embodiment, such fertilized egg may carry a paternal unlabeled Z chromosome and a maternal unlabeled Z chromosome, and therefore may carry an unlabeled male embryo. In some embodiments, such egg may be referred to herein as an unlabeled egg or as a non-transgenic egg (or WT or normal egg).

It should be understood that in case of transgenic fertilized eggs, specifically, eggs laid by or fertilized by a transgenic avian subject provided by the invention, that carry the reporter gene of the invention integrated into a gender chromosome thereof, in some embodiments the reporter gene is integrated in the transgenic egg at the same locus as in the transgenic animal laid or fertilized such egg.

In some specific embodiments, the at least one reporter gene may be integrated into the gender chromosome of the transgenic animal laid or fertilized the egg of the invention using at least one PEN. More specifically, such PEN may be in certain embodiments, a CRISPR type II system.

In yet more specific embodiments, the at least one reporter gene may be integrated into the gender chromosome of the transgenic avian animal laid or fertilized the egg/s of the invention by HDR mediated by at least one CRISPR/Cas9 system.

In more specific embodiments, the at least one reporter gene may be integrated into a gender chromosome of the transgenic avian animal laid or fertilized the egg/s of the invention by co-transfecting at least one cell of this avian animal, or at least one cell that is to be introduced into said avian animal with at least two nucleic acid sequences. More specifically, such cell may be co-transfected with (a) at least one first nucleic acid sequence comprising at least one nucleic acid sequence encoding at least one Cas9 protein and at least one nucleic acid sequence encoding at least one gRNA, thereby providing a CRISPR mediated integration; and (b) at least one second nucleic acid sequence comprising at least one reporter gene.

In more specific embodiments, the at least one reporter gene in the second nucleic acid sequence may be flanked at 5′ and 3′ thereof by homologous arms. These arms exhibit homology to the integration target site within the target gender chromosome, thereby facilitating HDR at the integration site.

In yet more specific embodiments, the at least one reporter gene in the second nucleic acid sequence may be operably linked to any one of a gender specific promoter, an embryonal specific promoter and an inducible promoter. Such promoter should limit the expression of the reporter gene of the invention to the specific desired gender (in case of gender specific promoter), the specific embryonic stage (embryonic specific promoter) or specific conditions (inducible conditions).

In yet some further specific embodiments, the at least one reporter gene comprised within the transgenic avian animal laid or fertilized the egg/s of the invention may be integrated into at least one non-coding region of one of its gender chromosomes.

In certain embodiments, the at least one reporter gene may be integrated into at least one site at gender W chromosome. In some particular embodiments, the integration site may be located at locus 1022859-1024215 at the W chromosome, specifically, galGal5_dna range of chromosome W:1022859-1024215. In yet some further specific embodiments, such loci comprises the nucleic acid sequence as denoted by SEQ ID NO. 3.

For specific integration of the reporter gene of the invention at any position within the loci described above, specific gRNAs may be required. Therefore, in some particular and non-limiting embodiments, appropriate gRNAs used for the preparation of the transgenic avian animal laid or fertilized the egg/s of the invention may comprise the nucleic acid sequence as denoted by any one of SEQ ID NO. 1 and 2. In some specific embodiments, these gRNAs are referred to herein as gRNA1 and gRNA2, respectively.

In some particular embodiments, the transgenic avian animal laid or fertilized the egg/s of the invention has been prepared using a gRNA1 that comprises the nucleic acid sequence as denoted by SEQ ID NO. 1. To enable integration of the reporter gene of the invention in such specific location, the reporter gene that should be integrated, must carry in certain embodiments, particular arms facilitating incorporation thereof in the target integration site directed by the gRNA used. Thus, in some specific embodiments, the at least one reporter gene may be comprised within the second nucleic acid sequence, where this reporter gene is flanked at 5′ and 3′ thereof by homologous arms comprising the amino acid sequence as denoted by SEQ ID NO. 4 and 5, respectively.

In yet some alternative embodiments, the transgenic avian animal laid or fertilized the egg of the invention may be prepared using a gRNA2 that comprises the nucleic acid sequence as denoted by SEQ ID NO. 2. In such case, to enable integration of the reporter gene of the invention at the specific site recognized by said gRNA2, the at least one reporter gene comprised within the second nucleic acid sequence may be according to specific embodiments, flanked at 5′ and 3′ thereof by homologous arms comprising the amino acid sequence as denoted by SEQ ID NO. 6 and 7, respectively.

In yet some further alternative embodiments, the transgenic avian animal laid or fertilized the egg/s of the invention may comprise at least one reporter gene integrated into at least one site at gender Z chromosome. In some particular and non-limiting embodiments, such avian transgenic animal may be female that carry the transgenic reporter gene integrated into the Z chromosome. In more specific embodiments, the specific loci in the Z chromosome may be any one of regions 9156874-9161874, as denoted by SEQ ID NO:15, 27764943-27769943, as denoted by SEQ ID NO:16, 42172748-42177748, as denoted by SEQ ID NO:17, 63363656-63368656, as denoted by SEQ ID NO:18 and 78777477-78782477, as denoted by SEQ ID NO:19 of Chromosome Z of female chicken.

In more specific embodiments, the at least one gRNA required to target the reporter gene to such specific location within the Z chromosome may comprises the nucleic acid sequence as denoted by any one of gRNA3: ACAGACCTATGATATGT, as denoted by SEQ ID NO. 11; gRNA4: CGATTATCACTCACAAG, as denoted by SEQ ID NO. 12; gRNA5: CTGGTTAGCATGGGGAC, as denoted by SEQ ID NO. 13 ; gRNA6: GTAAAGAGTCAGATACA, as denoted by SEQ ID NO. 14.

In yet some further embodiments, for integrating the reporter gene of the invention into the specific locus within the Z chromosome, left arm comprising the nucleic acid sequence as denoted by SEQ ID NO. 41, and right arm comprising the nucleic acid sequence as denoted by SEQ ID NO. 42, may be used to integrate the reporter gene of the invention to the specific loci directed by gRNA3 of SEQ ID NO:11. In further embodiments, for integrating the reporter gene of the invention into the specific locus within the Z chromosome, left arm comprising the nucleic acid sequence as denoted by SEQ ID NO. 43, and right arm comprising the nucleic acid sequence as denoted by SEQ ID NO. 44, may be used to integrate the reporter gene of the invention to the specific loci directed by gRNA4 of SEQ ID NO:12. In still further embodiments, for integrating the reporter gene of the invention into the specific locus within the Z chromosome, left arm comprising the nucleic acid sequence as denoted by SEQ ID NO. 45, and right arm comprising the nucleic acid sequence as denoted by SEQ ID NO. 46, may be used to integrate the reporter gene of the invention to the specific loci directed by gRNA5 of SEQ ID NO:13. In some further embodiments, for integrating the reporter gene of the invention into the specific locus within the Z chromosome, left arm comprising the nucleic acid sequence as denoted by SEQ ID NO. 47, and right arm comprising the nucleic acid sequence as denoted by SEQ ID NO. 48, may be used to integrate the reporter gene of the invention to the specific loci directed by gRNA6 of SEQ ID NO:14.

Further non-limiting examples for gRNA sequences suitable for integration into specific loci within the Z chromosome, may include but are not limited to gRNA7 of Z chromosome locus chrZ_42174515_-1, comprising the nucleic acid sequence GTAATACAGAGCTAAACCAG, as also denoted by SEQ ID NO:26, gRNA8 of Z chromosome locus chrZ_9157091_1, comprising the nucleic acid sequence ACAGACCTATGATATGTGAG, as also denoted by SEQ ID NO:27, gRNA9 of Z chromosome locus chrZ_27767602_-1, comprising the nucleic acid sequence GAGCTTGTGAGTGATAATCG, as also denoted by SEQ ID NO:28, gRNA10 of Z chromosome locus chrZ_78779927_1, comprising the nucleic acid sequence GTAAAGAGTCAGATACACAG, as also denoted by SEQ ID NO: 29, and gRNA11 of Z chromosome locus chrZ_63364946_-1, comprising the nucleic acid sequence CAGTGGGTACTGAAGCTGTG as also denoted by SEQ ID NO: 30.

In yet some further embodiments in accordance with the invention, for integrating the reporter gene of the invention into the specific locus within the Z chromosome, left arm comprising the nucleic acid sequence as denoted by SEQ ID NO. 31, and right arm comprising the nucleic acid sequence as denoted by SEQ ID NO. 32, may be used to integrate the reporter gene of the invention to the specific loci directed by gRNA7 of SEQ ID NO:26. In further embodiments, for integrating the reporter gene of the invention into the specific locus within the Z chromosome, left arm comprising the nucleic acid sequence as denoted by SEQ ID NO. 33, and right arm comprising the nucleic acid sequence as denoted by SEQ ID NO. 34, may be used to integrate the reporter gene of the invention to the specific loci directed by gRNA8 of SEQ ID NO:27. In still further embodiments, for integrating the reporter gene of the invention into the specific locus within the Z chromosome, left arm comprising the nucleic acid sequence as denoted by SEQ ID NO. 35, and right arm comprising the nucleic acid sequence as denoted by SEQ ID NO. 36, may be used to integrate the reporter gene of the invention to the specific loci directed by gRNA9 of SEQ ID NO:28. In further embodiments, for integrating the reporter gene of the invention into the specific locus within the Z chromosome, left arm comprising the nucleic acid sequence as denoted by SEQ ID NO. 37, and right arm comprising the nucleic acid sequence as denoted by SEQ ID NO. 38, may be used to integrate the reporter gene of the invention to the specific loci directed by gRNA10 of SEQ ID NO:29. In yet a further embodiment, for integrating the reporter gene of the invention into the specific locus within the Z chromosome, left arm comprising the nucleic acid sequence as denoted by SEQ ID NO. 39, and right arm comprising the nucleic acid sequence as denoted by SEQ ID NO. 40, may be used to integrate the reporter gene of the invention to the specific loci directed by gRNA11 of SEQ ID NO:30.

Still further, in some embodiments, the egg/s of the invention may be laid by a progeny of any of the transgenic avian subjects provided by the invention. In accordance with such embodiments, the egg/s of the invention may be either a fertilized or a non-fertilized egg. In some particular embodiments, when selection of female egg laying avian subject is desired, a transgenic avian female that contains the reporter gene of the invention integrated into its Z gender chromosome thereof may be used. A fertilized egg laid by such transgenic avian female (fertilized either by a WT or by a transgenic avian male) may be determined by the method of the invention as containing a female embryo if no detectable signal of the reporter gene is detected (in other words, the Z chromosome of such embryo is a paternal Z chromosome). Such egg may be further incubated and a female egg-laying avian subject may be successfully hatched and developed. It should be appreciated that such female avian subject is considered by the invention as a progeny of the transgenic avian subjects provided by the invention, and therefore, the present invention further encompasses any egg produced by such egg-laying hen and any components, parts and products thereof. It should be understood that such egg may be either fertilized or non-fertilized egg/s. In yet some further alternative embodiments, in case of a male embryo progeny of such fertilization (a male that carry a maternal Z chromosome having the reporter gene of the invention integrated therein), any egg/s laid by a hen fertilized by such male progeny, is also encompassed by the present invention, as well as any components, products and uses thereof. Still further, any progeny hatched from an egg laid by a transgenic female avian subject that carry the reporter gene of the invention integrated into its W chromosome thereof (either a male that carry two unlabeled Z chromosomes, or a female that carry the maternal labeled W chromosome) are considered herein as progenies of the transgenic avian subjects of the invention and as such, any egg/s laid by such avian subjects or fertilized by such progenies, may be also encompassed by the invention, as well as any parts, components and products thereof. Similarly, in some further embodiments, when a transgenic avian subject used is a male having the reporter gene integrated into at least one Z chromosome thereof, any egg laid by a female progeny, or fertilized by a male progeny of such transgenic male, is also encompassed by the invention.

Still further, it should be noted that the present invention further encompasses any egg product or any product that contains or prepared using the eggs of the invention or any components thereof (e.g., egg parts, specifically, egg shell, membrane, white and yolk, as well as any proteins, lipids or any substances comprised therein), or prepared by a process involving or using any of the eggs of the invention or any components thereof. The term “egg products” refers to any product/s obtained from eggs, from their different components or blends, once the shell and membranes have been removed and that are destined for human consumption or any other use described herein. This term includes eggs that are removed from their shells for processing and convenience, for commercial, foodservice, and home use. These products can be classified as refrigerated liquid, frozen, and dried products.

They can be partially complemented by other food products or additives and can be found solid, concentrated, liquid, dried, crystallized, frozen, deep-frozen or coagulated.

The possibilities in the use of egg products in accordance with the invention, are varied due to the techno-functional properties that they provide. Such properties may include foaming, emulsifying, and a unique color and flavor, which are important in several industrial products and processes, to name but a few, Confectionery, Bakery, Pastry, Dairy products, Ice creams, Drinks, Baby food, Creams and soups, Mayonnaise and sauces, Pasta, Ready cooked meals, Delicatessen, Pet food, Fish farming food, Cosmetic products, Glues (specifically, albumin), Tannery, pains, Pharmaceutical Industry. Still further, egg components and parts may also display useful properties and any uses thereof is also encompassed by the invention. More specifically, egg yolk and components thereof, may exhibit variety of properties such as, Flavouring, Colouring (by Xanthophyllis), Emulsifier capacity (by Lecithin, Lipoproteins LDL), Coagulant and binding substance (by Lipoproteins LDL and other proteins), Antioxidant (Phosvitin), Pharmaceutical uses (IgY, Cholesterol, Sialic acid). Egg white and its main protein, albumen may display Frother capacity, foam stabilizer (Lysozyme, Egg albumen), Anticrystallization (Egg mucin, Egg mucoid), Coagulant and binding substance (by Egg Albumin, Conalbumin), Preservatives (Lysozyme, Conalbumin), Rheological properties and Pharmaceutical properties.

In some embodiments, any of the eggs of the invention as disclosed herein or any component, element part or product thereof may be used for cosmetic applications. More specifically, egg white produced from the eggs of the invention may be used as a facial products, skin care, hair care and in lotions. Egg yolks produces from any of the eggs of the invention may be used in shampoos, conditioners and soaps. Cholesterol, lecithin and some of the egg's fatty acids may be used in skin care products, such as revitalizers, make-up foundations and lipstick.

In yet some further embodiments, the eggs of the invention may be used in animal feed. The excellent nutrition of eggs enhances various pet foods. Egg white may be used as a protein reference in feeding laboratory animals. Eggshells produced from the eggs of the invention may be dried, crushed and used to fed to laying hens as a rich calcium source and high-quality protein source (from egg white left inside the shells).

In yet some further embodiments, any of the eggs of the invention as disclosed herein or any component, element part or product thereof may be used for medical and pharmaceutical application. More specifically, fertile eggs provided by the invention may be used to manufacture vaccines (including influenza shots), as a source of purified protein and as an aid in the preservation of bull semen for artificial insemination.

Still further in some embodiments, any of the eggs of the invention as disclosed herein or any component, element part or product thereof may be used for nutraceutical application. More specifically, particular components purified and prepared from the eggs of the invention may be specifically applicable, in different products and processes. For example, lysozyme, an egg white protein, may be used as a food preservative and as an antimicrobial agent in pharmaceutical products. Avidin that is an egg white protein and biotin that is a vitamin found in egg white and, to a much greater extent, in egg yolk, may be prepared and purified from any of the eggs of the invention. Avidin-biotin technology in accordance with the invention may be used in various medical diagnostic applications such as immuno-assay, histopathology and gene probes. Sialic acid, an amido acid, that may be purified from any of the eggs of the invention, has been shown to inhibit certain stomach infections. Liposomes, fatty droplets found in eggs, are used as a controlled delivery mechanism for various drugs Immunoglobulin yolk (IGY), a simple egg-yolk protein which has immunological properties, may be used as an anti-human-rotavirus (HRV) antibody in food products. Phosvitin, a phosphoprotein found in egg yolk, provides antioxidant benefits in food products. Choline, a B vitamin combined with lecithin in egg yolk, is important in brain development and is used to treat certain liver disorders. Eggs are one of the best food sources of choline. Ovolecithin, a phospholipid found in egg yolk, has a high proportion of phosphatidycholine and contains fatty acids—such as arachidonic acid (AA) and docosahexanoic acid (DHA), which have been shown to improve visual activity in infants and to improve fatty-acid status. Egg lecithin has both emulsifying and antioxidant properties and, beyond its usefulness in keeping the oil and vinegar of mayonnaise in suspension, it's used chiefly in medicine. Shell-membrane protein is being used experimentally to grow human skin fibroblasts (connective tissue cells) for severe-bum victims and in cosmetics.

In yet some further embodiments, the invention further provides the use of egg shells prepared from any of the eggs of the invention, as a dietary source of calcium for humans and other mammals. In further embodiments, these egg shells may be used as a powdered, purified product in fortification of breads and confectioneries, fruit drinks, crackers, condiments. Egg shell calcium in accordance with the invention may be also used as oral phosphate binder in low phosphate diets for e.g. patients suffering from renal failure.

Still further, in some embodiments thereof, the invention provides the use of any protein or substance separated and/or purified from any of the eggs of the invention or from any element or component thereof. More specifically, such separated proteins can be used in food and pharmaceutical industry as is or after enzymatic modifications. In some embodiments, ovotransferrin that may be separated from any of the eggs of the invention, may be used as a metal transporter, antimicrobial, or anticancer agent, whereas lysozyme may be mainly used as a food preservative, and ovalbumin may be used as a nutrient supplement. Ovomucoid may be used to as an anticancer agent and ovomucin as a tumor suppression agent. Hydrolyzed peptides from these proteins may be also used for anticancer, metal binding, and antioxidant activities. Therefore, separation of egg white proteins from any of the eggs of the invention and the productions of bioactive peptides from egg white proteins are all are encompassed by the present invention. In yet some more specific embodiments, lysozyme that may be separated from any of the eggs of the invention, may be used as a bacteriolytic protein. Lysozyme has the capability of controlling foodborne pathogens such as Listeria monocytogens and Clostridium botulinum, which are considered as the major pathogens in the food industry. Lysozyme effectively controls toxin formation by Clostridium botulinum in fish, poultry, and some vegetables. Lysozyme also display antiviral, anti-inflammatory, and therapeutic effects. In yet some further embodiments, lysozyme that may be separated from any of the eggs of the invention, may be used in food as a preservative (e.g., kimuchi pickles, sushi, Chinese noodles, cheese, and wine production). In yet some further embodiments, Ovotransferrin that may be separated from any of the eggs of the invention, have a strong antimicrobial activity, and therefore may be used to improve the safety of foods. In yet some further embodiments, Ovomucin, that may be separated from any of the eggs of the invention, display a strong antimicrobial effect against food poisoning bacteria and therefore may be used in food industry as a food preservative. Also, it has a good emulsifying and forming characteristics that are essential and therefore applicable in the bakery industry. Still further, Ovotransferrin that may be separated from any of the eggs of the invention, can bind iron and easily releases the bound iron and as such, may be used as a source of iron supplementation for humans.

In yet a further aspect, the invention provides a kit comprising:

(a) at least one first nucleic acid sequence comprising at least one nucleic acid sequence encoding at least one Cas9 protein and at least one nucleic acid sequence encoding at least one guide RNA (gRNA); and (b) at least one second nucleic acid sequence comprising at least one said reporter gene.

In some embodiments, the at least one reporter gene in the second nucleic acid sequence comprised within the kit of the invention, may be flanked at 5′ and 3′ thereof by homologous arms for HDR at the integration site.

In yet some further specific embodiments, the at least one reporter gene in the second nucleic acid sequence of the kit of the invention may be operably linked to any one of a gender specific promoter, an embryonic specific promoter and an inducible promoter.

In certain embodiments, the at least one reporter gene may be integrated into at least one non-coding region of the gender chromosome, specifically, to chromosome W. In such case, the first nucleic acid sequence of the kit of the invention may encode at least one gRNA comprising the nucleic acid sequence as denoted by any one of SEQ ID NO. 1 and 2, also referred to herein as gRNA1 and gRNA2, respectively.

In some specific embodiments, the first nucleic acid sequence of the kit of the invention may comprise a gRNA, being gRNA1. In some embodiments, such gRNA1 may comprise the nucleic acid sequence as denoted by SEQ ID NO. 1. In such case, the reporter gene comprised within said second nucleic acid sequence of the kit of the invention, may be flanked at 5′ and 3′ thereof by homologous arms comprising the amino acid sequence as denoted by SEQ ID NO. 4 and 5, respectively.

In yet some further alternative embodiments, the kit of the invention may comprise in the first nucleic acid sequence thereof, a sequence encoding gRNA2. In some specific embodiments, such sequence encodes the nucleic acid sequence as denoted by SEQ ID NO. 2. In yet some further embodiments, the least one reporter gene comprised within the second nucleic acid sequence of the kit of the invention, may be flanked at 5′ and 3′ thereof by homologous arms comprising the amino acid sequence as denoted by SEQ ID NO. 6 and 7, respectively.

In yet some further alternative embodiments, the at least one reporter gene may be integrated into at least one site at gender Z chromosome. In more specific embodiments, the specific loci in the Z chromosome may be any one of regions 9156874-9161874, as denoted by SEQ ID NO:15, 27764943-27769943, as denoted by SEQ ID NO:16, 42172748-42177748, as denoted by SEQ ID NO:17, 63363656-63368656, as denoted by SEQ ID NO:18 and 78777477-78782477, as denoted by SEQ ID NO:19 of Chromosome Z of female chicken.

Thus, in more specific embodiments, the first nucleic acid sequence of the kit of the invention may comprise a gRNA, being the at least one of gRNA3: ACAGACCTATGATATGT, as denoted by SEQ ID NO. 11; gRNA4: CGATTATCACTCACAAG, as denoted by SEQ ID NO. 12; gRNA5: CTGGTTAGCATGGGGAC, as denoted by SEQ ID NO. 13 ; gRNA6: GTAAAGAGTCAGATACA, as denoted by SEQ ID NO. 14.

In further embodiments, the at least one reporter gene comprised within the second nucleic acid sequence of the kit of the invention, may be flanked at 5′ and 3′ thereof by homologous arms comprising the amino acid sequence as denoted by any one of SEQ ID NO. 41-48. More specifically, for integrating the reporter gene of the invention into the specific locus within the Z chromosome, left arm comprising the nucleic acid sequence as denoted by SEQ ID NO. 41, and right arm comprising the nucleic acid sequence as denoted by SEQ ID NO. 42, may be used to integrate the reporter gene of the invention to the specific loci directed by gRNA3 of SEQ ID NO:11. In further embodiments, for integrating the reporter gene of the invention into the specific locus within the Z chromosome, left arm comprising the nucleic acid sequence as denoted by SEQ ID NO. 43, and right arm comprising the nucleic acid sequence as denoted by SEQ ID NO. 44, may be used to integrate the reporter gene of the invention to the specific loci directed by gRNA4 of SEQ ID NO:12. In still further embodiments, for integrating the reporter gene of the invention into the specific locus within the Z chromosome, left arm comprising the nucleic acid sequence as denoted by SEQ ID NO. 45, and right arm comprising the nucleic acid sequence as denoted by SEQ ID NO. 46, may be used to integrate the reporter gene of the invention to the specific loci directed by gRNA5 of SEQ ID NO:13. In some further embodiments, for integrating the reporter gene of the invention into the specific locus within the Z chromosome, left arm comprising the nucleic acid sequence as denoted by SEQ ID NO. 47, and right arm comprising the nucleic acid sequence as denoted by SEQ ID NO. 48, may be used to integrate the reporter gene of the invention to the specific loci directed by gRNA6 of SEQ ID NO:14.

Further non-limiting examples of the first nucleic acid sequence of the kit of the invention may comprise a gRNA, being the at least one of gRNA7 of Z chromosome locus chrZ_42174515_-1, comprising the nucleic acid sequence GTAATACAGAGCTAAACCAG, as also denoted by SEQ ID NO:26, gRNA8 of Z chromosome locus chrZ 9157091_1, comprising the nucleic acid sequence ACAGACCTATGATATGTGAG, as also denoted by SEQ ID NO:27, gRNA9 of Z chromosome locus chrZ_27767602_-1, comprising the nucleic acid sequence GAGCTTGTGAGTGATAATCG, as also denoted by SEQ ID NO:28, gRNA10 of Z chromosome locus chrZ_78779927_1, comprising the nucleic acid sequence GTAAAGAGTCAGATACACAG, as also denoted by SEQ ID NO: 29, and gRNA11 of Z chromosome locus chrZ_63364946_-1, comprising the nucleic acid sequence CAGTGGGTACTGAAGCTGTG as also denoted by SEQ ID NO: 30.

In further embodiments, the at least one reporter gene comprised within the second nucleic acid sequence of the kit of the invention, may be flanked at 5′ and 3′ thereof by homologous arms comprising the amino acid sequence as denoted by any one of SEQ ID NO. 31 to 40. More specifically, for integrating the reporter gene of the invention into the specific locus within the Z chromosome, left arm comprising the nucleic acid sequence as denoted by SEQ ID NO. 31, and right arm comprising the nucleic acid sequence as denoted by SEQ ID NO. 32, may be used to integrate the reporter gene of the invention to the specific loci directed by gRNA7 of SEQ ID NO:26. In further embodiments, for integrating the reporter gene of the invention into the specific locus within the Z chromosome, left arm comprising the nucleic acid sequence as denoted by SEQ ID NO. 33, and right arm comprising the nucleic acid sequence as denoted by SEQ ID NO. 34, may be used to integrate the reporter gene of the invention to the specific loci directed by gRNA8 of SEQ ID NO:27. In still further embodiments, for integrating the reporter gene of the invention into the specific locus within the Z chromosome, left arm comprising the nucleic acid sequence as denoted by SEQ ID NO. 35, and right arm comprising the nucleic acid sequence as denoted by SEQ ID NO. 36, may be used to integrate the reporter gene of the invention to the specific loci directed by gRNA9 of SEQ ID NO:28. In further embodiments, for integrating the reporter gene of the invention into the specific locus within the Z chromosome, left arm comprising the nucleic acid sequence as denoted by SEQ ID NO. 37, and right arm comprising the nucleic acid sequence as denoted by SEQ ID NO. 38, may be used to integrate the reporter gene of the invention to the specific loci directed by gRNA10 of SEQ ID NO:29. In yet a further embodiment, for integrating the reporter gene of the invention into the specific locus within the Z chromosome, left arm comprising the nucleic acid sequence as denoted by SEQ ID NO. 39, and right arm comprising the nucleic acid sequence as denoted by SEQ ID NO. 40, may be used to integrate the reporter gene of the invention to the specific loci directed by gRNA11 of SEQ ID NO:30.

In some embodiments, the reporter gene comprised within the second nucleic acid sequence of the kit of the invention may be at least one bioluminescence reporter gene.

In yet some further embodiments, the kit of the invention may be suitable for use in the preparation of a transgenic avian animal comprising at least one exogenous reporter gene integrated into at least one position or location in at least one of gender chromosome Z and W.

In some embodiments, the method of the invention may use any of the kits of the invention as described herein.

Still further, it must be appreciated that the kits of the invention may further comprise any reagent, buffer, media or material required for the preparation of the transgenic avian animals of the invention. The kit of the invention may further comprise instructions as well as containers for the different components thereof.

It should be appreciated that in certain embodiments, the oligonucleotide/s or polynucleotide/s used by the kit/s and method/s of the invention are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially” of means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989,1992), and in Ausubel et al., Current Protocols in Molecular Biology, John Wiley and Sons, Baltimore, Md. (1988).

Reagents

Animals:

Commercial White Leghorn chickens are obtained from Hendrix ISA, and Minnesota.

Marker Line chickens are from the Pacific Agri-Food Research Centre, Agassiz, British Columbia, Canada.

Transgenic mice CAG-luc-eGFP are from The Jackson Laboratory (catalogue number L2G85).

Transgenic mice C57BL/6-Tg(CAG-EGFP)1Osb/J are from The Jackson Laboratory (catalogue number 00329).

Animal experiments were done in strict accordance to IACUC approved protocols and under supervision of the Crystal Bioscience IACUC committee ensuring that no animal suffers from illness nor dies during the course of the experiments.

Vectors:

Cas9 SmartNuclease™ All-in-one tagged vectors is ordered from System Bioscience Inc., catalogue number CAS8/9xx series.

pCMV-Gluc 2 vector is ordered from New England Biolabs Inc., catalogue number N8081S.

Cell Lines:

Female cells are of Gallus gallus, chicken T lymphocyte cells.

Male cells are of Gallus gallus, chicken Liver, ordered from ATCC® Number: CRL-2118™.

Primordial Germ Cell (PGC) line.

Experimental Procedures

Reporter Gene Bioluminescence Detection Through the Egg Shell

D-Luciferin (Sigma-Aldrich Co. LLC, Israel, catalogue number 2591-17-5) is dissolved at room temperature in DPBS to a final concentration of 15 mg/mL.

An amount of 0.1 ml of luciferin or saline solution (negative control) is injected subcutaneously in the loose skin around the neck and shoulder area of transgenic luciferase-expressing mice. Ear and tail are excised after 10 min and introduced into Chicken embryo (10 days).

Alternatively, excised ear and tail from transgenic luciferase-expressing transgenic mice is incorporated into the Chicken embryo prior to direct injection to the egg of 0.1 ml of luciferin or saline solution.

Bioluminescence is observed by using Bio-space photon Imager (Bio space lab, USA).

Restriction-Free (RF) Cloning

The insertion of gRNAs into Cas9-SmartNuclease™ vector is performed by applying the Restriction Free method (Peleg Y et al., 2010). Primers are ordered from Sigma-Genosys (Rehovot, Israel) and subsequent RF reactions were carried out using Phusion polymerase (Thermo Scientific, Hudson, N.H., USA). Plasmid purification is carried out using the MEGAspin kit and DNA-spin plasmid DNA purification kit, respectively (Intron Biotechnology Biotechnology, Daejoen, South Korea).

Cell Culture

PGCs are grown in KO-DMEM (Life Technologies), of which 40% is preconditioned on buffalo rat liver cells (BRL, ATCC), and supplemented with 7.5% fetal bovine serum (Hyclone), 2.5% irradiated chicken serum, 1× non-essential amino acids, 2 mM glutamine, 1 mM sodium pyruvate, 0.1 mMβ-mercaptoethanol (all from Life Technologies), 4 ng/ml recombinant human fibroblast growth factor, 6 ng/ml recombinant mouse stem cell factor (both from R&D Systems) and grow on an irradiated feeder layer of BRL cells. The cells are passaged 3 times per week onto fresh feeder layers.

Transfection

For stable transfectant targeting of the above-mentioned loci of chromosomes W or Z, 15 μg of vector-containing gRNA and 15 μg of circular luciferase-containing vector were added to 5×10⁶ cells and brought to volume of 100 μl with V-buffer (lonza, Walkersville). The cell suspension is transferred to a 2 mm cuvette and subjected to 8 square wave pulses of 350 volts/100 μsec (BTX 830 electroporator). Cells are then plated with Neomycin-resistant irradiated BRLs and seeded in a 48-well plate at a density of 10⁵ cells per well. After 3 days, 40μg/ml Neomycin is added to select for cells with a stable integration of luciferase reporter gene.

Preparation of Transgenic Chickens

Concentrated vehicle (that may be either lentivirus at a titer of about 10⁷ MOI) or plasmid DNA) is injected to 25 embryos in new laid eggs. Injections are carried out weekly three injections. The injected embryos hatch 3 weeks after injection. These are G0 birds Immediately after hatch, the DNA is extracted from CAM samples of the hatched chicks and detection of the presence/absence of vector DNA is carried out by semi-quantitative PCR. Blood sample G0 chicks at 2-3 weeks of age and repeat PCR screen. G0 birds are raised to sexual maturity, 16-20 weeks for males, 20-24 weeks for females. Cockerels are tested for semen production from approximately 16 weeks.

Hens are inseminated, fertile eggs collected daily. The G1 chicks are hatch 3 weeks later and each individual chick wing banded and a chick chorioallantoic membrane (CAM) sample taken from the shell. Extract DNA from CAM samples and carry out PCR screen for presence of transgene, predicted to be single copy level. Repeat screen to confirm and sex chicks on DNA from blood sample 2-3 weeks later.

At a few weeks of age a blood sample is taken from G1 birds to prepare genomic DNA for PCR analysis. G1 birds are used for breeding G2.

### Example 1

### Selection of Reporter Gene for Visual Gender Identification in Poultry

In order to demonstrate the feasibility of visually identify gender of in-ovo poultry, the use of bioluminescent as compared to fluorescent reporter genes was evaluated. Therefore, transgenic mice expressing reporter genes such as firefly luciferase (having a nucleic acid sequence as denoted by SEQ ID NO:20; encoding the amino acid sequence as denoted by SEQ ID NO:21)and green fluorescent protein (eGFP), were first employed.

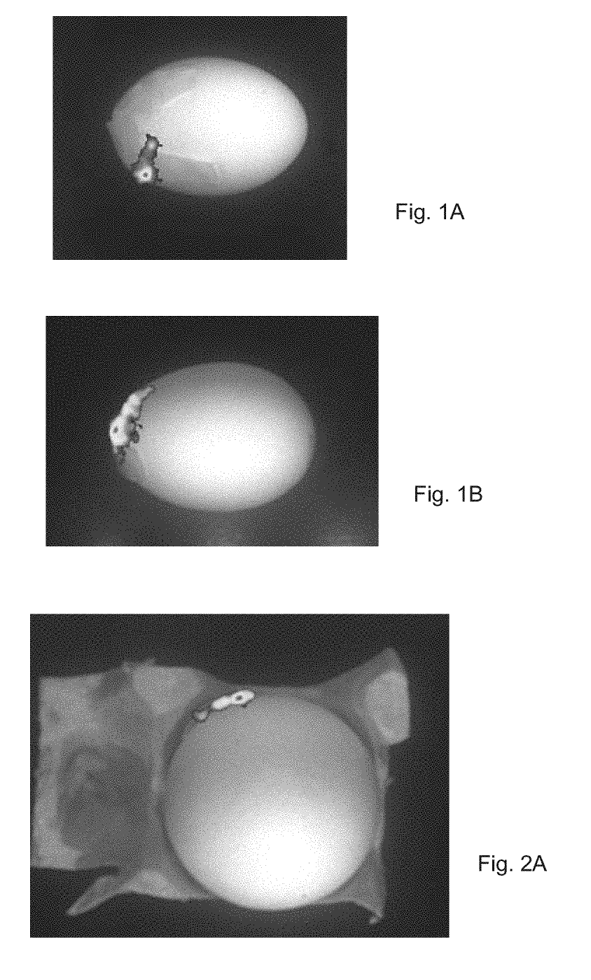

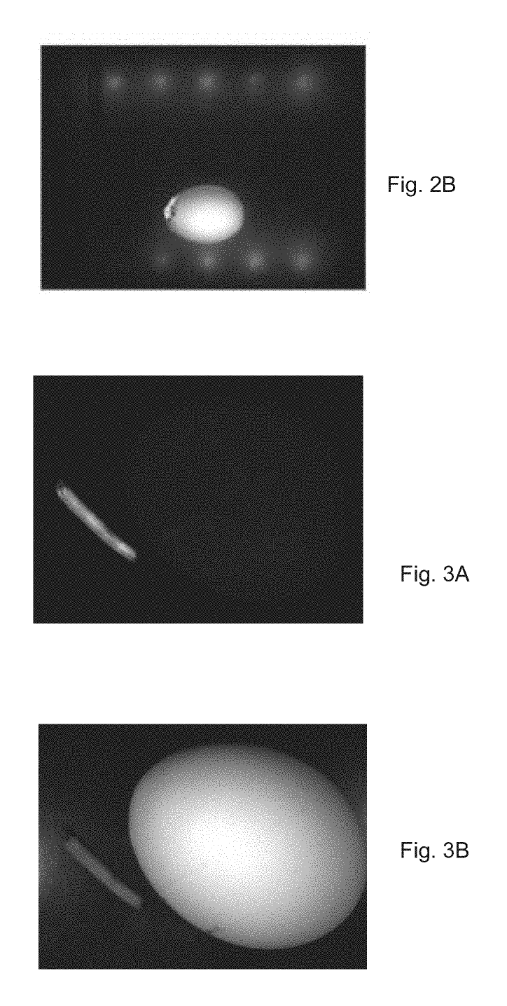

For observation of luciferase activity, in FIG. 1 luciferin was injected subcutaneously to luciferase-expressing transgenic mice, tails and ears were then excised and introduced through a 5 mm hole in the egg shell of an unfertilized egg. As shown in the figure, the luciferase detectable signal is clearly observed in tail and ear samples (FIGS. 1A, 1B) through the egg shell. The inventors therefore next examined the feasibility of inducing luciferase reaction in-ovo. Therefore, ears and tails of luciferase-expressing transgenic mice were excised, introduced through a hole into a fertilized egg that carry a 10-days old chicken embryo and luciferin was subsequently injected. As clearly shown in FIGS. 2A and 2B, an in-ovo luciferase reaction successfully resulted in a detectable signal that was able to penetrate the egg shell.

On the other hand, similar experiments performed using GFP as the reporter gene, clearly indicated that GFP signal is not detectable following incorporation of tails and ears of GFP-expressing transgenic mice into Chicken embryo as seen in FIG. 3.

Luciferase reporter gene, specifically, firefly luciferase (comprising the amino acid sequence as denoted by SEQ ID NO. 21, encoded by the nucleic acid sequence as denoted by SEQ ID NO:20) was thus further selected for incorporation into sex chromosomes W and Z.

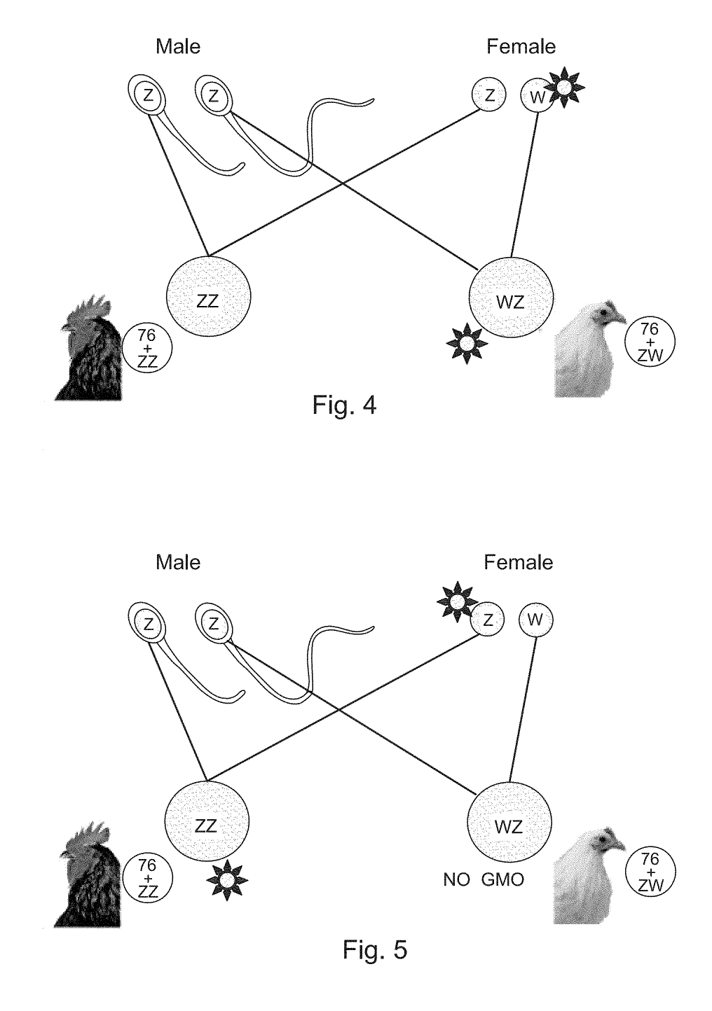

FIG. 4 represents a schematic illustration of the method of the invention for identification of embryo's gender in-ovo. More specifically, a transgenic avian female hen containing a gender specific chromosome (W) with the luciferase reporter gene integrated therein is provided. In eggs laid by said hen, expression of the reporter gene observed by a detectable signal indicates that the embryo carry the W gender chromosome and is therefore female. This enables the selection for continued incubation of male while females that carry the reporter gene are discarded. This selection is probably more relevant for Poultry.

FIG. 5 schematically presents yet a further alternative that facilitates determination of male embryo, in-ovo. More specifically, the provision of transgenic female chickens carrying the gender specific Z chromosome with a reporter gene integrated therein, results in female embryos (that received the maternal wild type W chromosome) without reporter gene or male embryos (that received the maternal labeled Z chromosome) expressing the transgenic luciferase gene.

### Example 2

### Design of Guide RNAs Vector

In order to incorporate the luciferase reporter gene into the gender chromosomes W or Z, the CRISPR/Cas9 mediated HDR method is selected. Relevant gRNA sites are then sought from both gender chromosomes.

The region 1022859-1024215 of Chromosome W of female chicken, comprising the nucleic acid sequence as denoted by SEQ ID NO. 3, is analyzed for guide RNA design. Two guide RNAs are selected, synthesized and cloned separately into the Cas9 SmartNuclease vector containing the wild type Cas9 nuclease (Horizon) by Restriction free cloning protocol: gRNA1: GCACTAGGAACCAGCAGCAG, as denoted by SEQ ID NO. 1 and gRNA2: GTAGCCCCAAGAGGGCTAGG, as denoted by SEQ ID NO. 2.

The predicted parameters of these two gRNAs are presented in Table 1:

TABLE 1

| gRNA parameters | gRNA1 | gRNA2 |
|---|---|---|
| sgRNA designer | 0.506 | 0.63 |
| sscore | 0.8677 | 0.5323 |
| sgRNA scorer | 94.8 | 99.9 |

The regions 9156874-9161874, as denoted by SEQ ID NO:15, 27764943-27769943, as denoted by SEQ ID NO:16, 42172748-42177748, as denoted by SEQ ID NO:17, 63363656-63368656, as denoted by SEQ ID NO:18 and 78777477-78782477, as denoted by SEQ ID NO:19 of Chromosome Z of female chicken are analyzed for guide RNA design. Four guide RNAs are selected, synthesized and cloned separately into the Cas9 SmartNuclease vector containing the wild type Cas9 nuclease (Horizon) by Restriction free cloning protocol: gRNA3: ACAGACCTATGATATGT, as denoted by SEQ ID NO. 11; gRNA4: CGATTATCACTCACAAG, as denoted by SEQ ID NO. 12; gRNA5: CTGGTTAGCATGGGGAC, as denoted by SEQ ID NO. 13 ; gRNA6: GTAAAGAGTCAGATACA, as denoted by SEQ ID NO. 14.

Further non-limiting examples for gRNA sequences suitable for integration into specific loci within the Z chromosome, may include but are not limited to gRNA7 of Z chromosome locus chrZ_42174515_-1, comprising the nucleic acid sequence GTAATACAGAGCTAAACCAG, as also denoted by SEQ ID NO:26, gRNA8 of Z chromosome locus chrZ_9157091_1, comprising the nucleic acid sequence ACAGACCTATGATATGTGAG, as also denoted by SEQ ID NO:27, gRNA9 of Z chromosome locus chrZ_27767602_-1, comprising the nucleic acid sequence GAGCTTGTGAGTGATAATCG, as also denoted by SEQ ID NO:28, gRNA10 of Z chromosome locus chrZ_78779927_1, comprising the nucleic acid sequence GTAAAGAGTCAGATACACAG, as also denoted by SEQ ID NO: 29, and gRNA11 of Z chromosome locus chrZ_63364946_-1, comprising the nucleic acid sequence CAGTGGGTACTGAAGCTGTG as also denoted by SEQ ID NO: 30.

These gRNAs have few predicted off-target sites, none of which were in known coding sequences.

### Example 3

Design of Luciferase Targeting Vector

Flanking sequences homological of the appropriate flanking sequences indicated above of female W chromosome or of the female Z chromosome loci, are introduced into the luciferase-expressing vector upstream to the CMV-promoter and downstream the Neomycin-resistance or alternatively downstream the polyA site (ordered synthetic DNA, Integrated DNA Technologies, Inc., USA).

For the female W chromosome, the reporter gene, specifically Luciferase may be cloned for using either the Guide 1 (gRNA1), as denoted by SEQ ID NO. 1 or Guide 2 (gRNA2): as denoted by SEQ ID NO. 2. For cloning using the gRNA1, “Left arm” comprising the nucleic acid sequence as denoted by SEQ ID NO. 4, and the “Right arm” comprising the nucleic acid sequence as denoted by SEQ ID NO. 5 are provided. For cloning using the gRNA2, “Left arm” comprising the nucleic acid sequence as denoted by SEQ ID NO. 6, and the “Right arm” comprising the nucleic acid sequence as denoted by SEQ ID NO. 7 are provided.

Still further, a “left arm” for the region upstream to the CMV-promoter comprises the nucleic acid sequence as denoted by SEQ ID NO. 8, and a “right arm” for the region downstream the Neomycin-resistance, may comprise the nucleic acid sequence as denoted by SEQ ID NO. 9, or SEQ ID NO.10 for the region downstream the polyA site. For the female Z chromosome, the Luciferase reporter gene may be cloned for using either the gRNA3, as denoted by SEQ ID NO. 11, gRNA4 : as denoted by SEQ ID NO. 12, gRNA5, as denoted by SEQ ID NO. 13, gRNA6, as denoted by SEQ ID NO. 14.

For cloning using the gRNA3, “Left arm” comprising the nucleic acid sequence as denoted by SEQ ID NO. 41, and the “Right arm” comprising the nucleic acid sequence as denoted by SEQ ID NO. 42 are provided. For cloning using the gRNA4, “Left arm” comprising the nucleic acid sequence as denoted by SEQ ID NO. 43, and the “Right arm” comprising the nucleic acid sequence as denoted by SEQ ID NO. 44 are provided. For cloning using the gRNA5, “Left arm” comprising the nucleic acid sequence as denoted by SEQ ID NO. 45, and the “Right arm” comprising the nucleic acid sequence as denoted by SEQ ID NO. 46 are provided. For cloning using the gRNA6, “Left arm” comprising the nucleic acid sequence as denoted by SEQ ID NO. 47, and the “Right arm” comprising the nucleic acid sequence as denoted by SEQ ID NO. 48 are provided. In yet some further embodiments, for integrating the reporter gene of the invention into the specific locus within the Z chromosome, left arm comprising the nucleic acid sequence as denoted by SEQ ID NO. 31, and right arm comprising the nucleic acid sequence as denoted by SEQ ID NO. 32, may be used to integrate the reporter gene of the invention to the specific loci directed by gRNA7 of SEQ ID NO:26. In further embodiments, for integrating the reporter gene of the invention into the specific locus within the Z chromosome, left arm comprising the nucleic acid sequence as denoted by SEQ ID NO. 33, and right arm comprising the nucleic acid sequence as denoted by SEQ ID NO. 34, may be used to integrate the reporter gene of the invention to the specific loci directed by gRNA8 of SEQ ID NO:27. In still further embodiments, for integrating the reporter gene of the invention into the specific locus within the Z chromosome, left arm comprising the nucleic acid sequence as denoted by SEQ ID NO. 35, and right arm comprising the nucleic acid sequence as denoted by SEQ ID NO. 36, may be used to integrate the reporter gene of the invention to the specific loci directed by gRNA9 of SEQ ID NO:28. In further embodiments, for integrating the reporter gene of the invention into the specific locus within the Z chromosome, left arm comprising the nucleic acid sequence as denoted by SEQ ID NO. 37, and right arm comprising the nucleic acid sequence as denoted by SEQ ID NO. 38, may be used to integrate the reporter gene of the invention to the specific loci directed by gRNA10 of SEQ ID NO:29. In yet a further embodiment, for integrating the reporter gene of the invention into the specific locus within the Z chromosome, left arm comprising the nucleic acid sequence as denoted by SEQ ID NO. 39, and right arm comprising the nucleic acid sequence as denoted by SEQ ID NO. 40, may be used to integrate the reporter gene of the invention to the specific loci directed by gRNA11 of SEQ ID NO:30.

### Example 4

### Germline Transmission of CRISPR-Treated Cells

The two above described vectors, specifically, the gRNA/Cas9 and the reporter-gene vectors are co-transfected to PGCs as detailed in experimental procedures. After stable clones are identified, the cells are expanded and confirmed for the luciferase integration by PCR. Confirmed clones are injected into recipient chicken embryos at Stage 14-16 (H&H). The injected embryos are transferred to surrogate shells and incubated until hatch at 37° C. The sex of the chicks is determined after hatch by PCR for the W-chromosome.

Female and Male chimeras are grown to sexual maturity and bred to wild type male and female chickens. Hatched chicks are evaluated for the expression of luciferase, and the germline progeny are confirmed by PCR to carry targeted luciferase.

1. A method of gender determination of avian fertilized unhatched egg, the method comprising the step of:
(a) providing or obtaining at least one transgenic avian animal comprising at least one exogenous reporter gene integrated into at least one position or location in at least one of gender chromosome Z and W;
(b) obtaining at least one fertilized egg from said transgenic avian subject, or of any cells thereof;
(c) determining in said egg if at least one detectable signal is detected, wherein detection of said at least one detectable signal indicates the expression of said at least one reporter gene, thereby the presence of said W chromosome or Z chromosome in said avian embryo.

2. The method according to claim 1, wherein said reporter gene is at least one bioluminescence reporter gene.

3. The method according to claim 2, wherein said reporter gene is luciferase.

4. The method according to claim 3, wherein said method further comprises the step of providing to said egg of step (b), at least one of substrate and enzyme compatible to said bioluminescence reporter gene, for formation of said detectable signal detected at step (c).

5. The method according to claim 1, wherein said at least one transgenic avian subject is a female avian animal, and:
(a) wherein said at least one reporter gene is integrated into at least one position of female chromosome Z, thereby detection of a detectable signal indicates that said embryo in said unhatched egg is a male; or
(b) wherein said at least one reporter gene is integrated into at least one position of female chromosome W, thereby detection of a detectable signal, indicates that said embryo in said unhatched egg is female.

6. The method according to claim 1, wherein said at least one reporter gene is integrated into said gender chromosome of said transgenic avian subject using at least one programmable engineered nuclease (PEN), and wherein said PEN is a clustered regularly interspaced short palindromic repeat (CRISPR) type II system.

7. The method according to claim 6, wherein said at least one reporter gene is integrated into said gender chromosome of said transgenic avian animal by homology directed repair (HDR) mediated by at least one CRISPR/CRISPR-associated endonuclease 9 (Cas9) system, and wherein said at least one reporter gene is integrated into a gender chromosome of said transgenic avian animal by co-transfecting at least one cell of said avian animal or at least one cell introduced into said avian animal, with:
(a) at least one first nucleic acid sequence comprising at least one nucleic acid sequence encoding at least one Cas9 protein and at least one nucleic acid sequence encoding at least one guide RNA (gRNA); and
(b) at least one second nucleic acid sequence comprising at least one said reporter gene.

8. The method according to claim 7, wherein said at least one reporter gene is integrated into:
(a) at least one site at gender W chromosome locus 1022859-1024215; or
(b) at least one site at gender Z chromosome locus 42172748-42177748.

9. An avian transgenic animal comprising at least one exogenous reporter gene integrated into at least one locus in at least one of gender chromosome Z and W.

10. The transgenic animal according to claim 9, wherein said reporter gene is at least one bioluminescence reporter gene.

11. The transgenic animal according to claim 10, wherein said reporter gene is luciferase.

12. The transgenic animal according to claim 9, wherein said at least one transgenic avian animal is a female, and:
(a) wherein said at least one reporter gene is integrated into at least one position of female chromosome Z; or
(b) wherein said at least one reporter gene is integrated into at least one position of female chromosome W.

13. The transgenic animal according to claim 9, wherein said at least one reporter gene is integrated into said gender chromosome of said transgenic avian animal by HDR mediated by at least one CRISPR/Cas9 system, and wherein said at least one reporter gene is integrated into a gender chromosome of said transgenic avian subject, specifically animal by co-transfecting at least one cell of said avian animal or at least one cell introduced into said avian animal:
(a) at least one first nucleic acid sequence comprising at least one nucleic acid sequence encoding at least one Cas9 protein and at least one nucleic acid sequence encoding at least one gRNA; and
(b) at least one second nucleic acid sequence comprising at least one said reporter gene.

14. The transgenic animal according to claim 9, wherein said at least one reporter gene is integrated into:
(a) at least one site at gender W chromosome locus 1022859-1024215; or
(b) at least one site at gender Z chromosome locus 42172748-42177748.

15. A cell comprising at least one exogenous reporter gene integrated into at least one locus in at least one of gender chromosome Z and W.

16. The cell according to claim 15, wherein said cell is an avian cell, and wherein said avian cell is a primordial germ cell (PGC).

17. The cell according to claim 15, wherein said at least one reporter gene is luciferase, and wherein said reporter gene is integrated into:
(a) at least one site at gender W chromosome locus 1022859-1024215; or
(b) at least one site at gender Z chromosome locus 42172748-42177748.

18. An egg derived, laid or fertilized by at least one transgenic avian subject or by any progeny thereof, any component or any parts thereof or any product comprising said egg, components or parts thereof, wherein said at least one transgenic avian subject comprises, in at least one cell thereof, at least one exogenous reporter gene integrated into at least one position in at least one of gender chromosome Z and W.

19. The egg according to claim 18, wherein said at least one transgenic avian animal is a female, and:
(a) wherein said at least one reporter gene is integrated into at least one position of female chromosome Z; or
(b) wherein said at least one reporter gene is integrated into at least one position of female chromosome W.

20. A kit comprising:
(a) at least one first nucleic acid sequence comprising at least one nucleic acid sequence encoding at least one Cas9 protein and at least one nucleic acid sequence encoding at least one guide RNA (gRNA); and
(b) at least one second nucleic acid sequence comprising at least one said reporter gene.
